(19) United States
(12) Patent Application Publication
(10) Pub. No.: US 2011/0206740 A1
(43) Pub. Date: Aug. 25, 2011

(54) Cell Membrane Engineering

(76) Inventors: Jeffrey M. Karp, Brookline, MA (US); Debanjan Sarkar, Williamsville, NY (US); Praveen Kumar Vemula, Cambridge, MA (US)

(21) Appl. No.: 12/990,021

(22) PCT Filed: Apr. 29, 2009
(86) PCT No.: PCT/US09/42087

§ 371 Date: May 10, 2011

Related U.S. Application Data
(60) Provisional application No. 61/048,773, filed on Apr. 29, 2008.

(51) Int. Cl. A61K 9/14; C12N 5/071; A61K 35/12; A61K 39/385; A61P 37/04

(52) U.S. Cl. 424/400; 435/325; 435/375; 424/93.7; 424/193.1

(57) ABSTRACT

The present invention is directed to methods and compositions for engineering the surface of a cell, wherein a targeting moiety and/or a particle are attached to the cell membrane. The particle can further comprise a therapeutic agent for drug delivery. The compositions disclosed herein are useful in the treatment of diseased or damaged tissue by targeting cells for the purpose of tissue regeneration, drug delivery or a combination of both.

### CROSS REFERENCE TO RELATED APPLICATION

This application claims benefit under 35 U.S.C. §119(e) of the U.S. Provisional Application No. 61/048,773, filed Apr. 29, 2008, the content of which is incorporated herein by reference in its entirety.

### FIELD OF THE INVENTION

The present invention relates to the field of targeted cell delivery to promote regeneration.

### BACKGROUND

Cells are important tools for therapeutics for different biomedical applications, including tissue specific drug delivery. For example, bacterial cells loaded with various anticancer drugs (doxorubicin, platin etc.) have been specifically targeted to cancer cells¹. Another example of cells as delivery vehicles is the use of genetically transduced human mesenchymal stem cell (MSC) for molecular engineering techniques to treat human deficiencies and diseases, as well as the delivery of appropriate chemotherapeutics. MSCs transduced by adenoviral expression vector carrying interferon-β (IFN-β) have been used to deliver IFN-β either intravenously or subcutaneously into malignant cancer cells to inhibit tumor growth². Similarly, CRAds (conditionally replicating adenoviruses) were targeted to malignant tumor cells through MSC by using mouse xenograft models³. These results indicate that CRAd transduced stem cells are homed to the tumor site more effectively than CRAds alone. MSC's are also known to possess homing capacity to bone⁴ and several other tissues⁵.

Macrophages are another example of an attractive cell-based carrier, for example in cancer treatment due to their ability to concentrate at tumor sites, kill tumor cells and inhibit tumor growth e.g. macrophages transduced to express CY2BP6 were able to kill tumor cells upon infiltration into the tumor spheroid in a hypertoxic environment⁶. Tumor specific T-cells (e.g. tumor infiltrating lymphocytes) are also used to home to tumors for therapeutic purposes e.g. murine T-cells transduced with MOv-γ gene were administered intra-percutaneously into mice with human ovarian cancer cells⁷. But in spite of these successful applications of cells, many studies have also indicated that proper targeting is not achieved in many cases⁸. The uncontrolled release of the therapeutics from T-cells at non-targeted and non-specific sites has led to a substantial degree of toxicity and risk to patient's health. Similarly, a pro-tumorigenic role of certain macrophages at the tumor sites has been reported⁹.

Cells are an indispensable part of the regeneration process in tissue engineering applications. The use of genetically modified cells and/or appropriate genes play increasingly significant roles in tissue regeneration. Genetically modified polymer matrices (both by polymeric release and substrate-mediated release) release particular genetic information to aid the tissue regeneration process¹⁰. The use of cells for engineering of tissues in scaffolds supplemented with growth factors enhances the regeneration of the tissue^{11,12}.

However, complications associated with these methods include: uncontrolled release, inappropriate level of gene expression, and aberrant tissue growth. Delivery of appropriate genes for directed regeneration of tissues is also achieved through transferring a desired gene into the cell using non-viral or viral vectors and subsequently delivering the cells for tissue regeneration¹³. Autologous cell therapy with systemic administration of MSCs is a powerful therapeutic tool for tissue regeneration¹⁴⁻¹⁶. However, MSCs cultured ex vivo lose their capacity to home to spleen and bone marrow due to loss of gene expression¹⁴.

Defining specific interactions between the cell and its environment has been an active area of research for successful functioning of therapeutics. The structure and functionality of the cell membrane permits the cell to interact with the extracellular matrix (ECM) through various interactions with substantial cross-talk, and may lead to cell adhesion, growth, migration, differentiation, matrix production, protease secretion, or apoptosis. As a consequence, the cell membrane is important for mediating interactions of a cell with its surroundings. The cell membrane (e.g., plasma membrane) is a semi-permeable lipid bilayer that contains a wide variety of biological molecules, primarily proteins and lipids, which are involved in a vast array of cellular processes, and also serves as the attachment point for the intracellular cytoskeleton. Aside from interactions with ECM, the cell membrane serves as a gateway for ions, small molecules and larger entities such as viruses. Studies with Murine Leukemia Virus (MLV) and Human Immune Deficiency Virus (HIV) particles show that the virus particles initially interact and bind to the cell surface¹⁷.

In addition to providing a gateway to the cell, the membrane actively mediates interactions with the surrounding. For example, cells capable of migrating through the extracellular matrix typically secrete proteases such as MMPs through the cell membrane, and certain cell types contain MMPs within the cell membrane (Membrane type-1 matrix metalloproteinase (MT1-MMP))¹⁸. Cancer cells with enhanced secretion of proteases also have an advanced ability to invade extracellular matrices¹⁹. Cells that have been modified via genetic modifications to enhance production of proteases²⁰, or cell surface receptors involved in proteolysis²¹, have been demonstrated to have enhanced invasion potential. This indicates that by proper modification of the cell surface, it is possible to modulate a cell's interaction with its environment.

Since the cell surface mediates interaction with the external environment and represents a substrate which encompasses complex interactions, it is desirable to exhibit greater control of cell membrane mediated interactions to produce bio-specific effects.

### SUMMARY OF THE INVENTION

In general, the invention described herein provides methods for functionalizing the cell surface by various techniques and for use in a range of applications. Unlike targeted drug delivery with nanoparticles and unlike delivery of unmodified cells on or within biomaterial based beads, the modification of a cell surface with a range of functionalities represents a new approach that facilitates enhanced control to manipulate cellular events for a range of applications. A functionalized cell surface can effectively be used to control practically any biological system. More specifically, the compositions and methods described herein are useful for targeting cells to tissues for regeneration, or alternatively for the cell-based delivery of agents to a particular tissue of interest. Cells useful for the methods described herein can be modified by the addition of a particle, as well as a ligand. The particle may further comprise an agent, such as a protein, a small molecule, an RNA interference molecule, a drug, a vitamin, a therapeutic agent, a diagnostic agent a nutraceutical, an agent that has cosmetic properties, or a nucleic acid. In addition, the compositions described herein can be tailored by one of skill in the art for the treatment of a wide range of diseases or wounds, including for example, stroke, organ regeneration, cancer, fractures, and ischemic heart disease, among others.

In one aspect, the invention relates to an isolated and engineered cell composition comprising a cell, a membrane associated ligand attached to a surface of the cell, and a particle attached to the surface of the cell.

In one embodiment of this aspect and all other aspects described herein, the cell is a stem cell or a progenitor cell.

In another embodiment of this aspect and all other aspects described herein, the stem cell is a reprogrammed cell.

In another embodiment of this aspect and all other aspects described herein, the cell is a differentiated cell.

In another embodiment of this aspect and all other aspects described herein, the cell is genetically engineered to express a therapeutic agent.

In another embodiment of this aspect and all other aspects described herein, the particle is 1000-8000 nm. Alternatively, the particle is 500-1000 nm or 1-500 nm.

In another embodiment of this aspect and all other aspects described herein, the membrane associated ligand is attached with a linker molecule.

Similarly, in another embodiment of this aspect and all other aspects described herein, the particle is attached with a linker molecule. Alternatively, in another embodiment the particle is attached without a linker molecule.

In another embodiment of this aspect and all other aspects described herein, the membrane associated ligand is attached covalently. Alternatively, in another embodiment the membrane associated ligand is attached non-covalently.

In another embodiment of this aspect and all other aspects described herein, the particle is attached covalently. Alternatively, in another embodiment the particle is attached non-covalently.

In another embodiment of this aspect and all other aspects described herein, the membrane associated ligand confers accumulation of the cell in a tissue.

In another embodiment of this aspect and all other aspects described herein, the membrane associated ligand is selected from a group consisting of an antibody, an antibody fragment, an aptamer, a peptide, a targeting moiety, a vitamin, a drug, a nutraceutical, a carbohydrate, a protein, a receptor, an adhesion molecule, a glycoprotein, a sugar residue, a therapeutic agent, a glycosaminoglycan, or any combination thereof.

In another embodiment of this aspect and all other aspects described herein, two or more membrane associated ligands are attached to the cell.

In another embodiment of this aspect and all other aspects described herein, the particle comprises an agent that enhances function of the cell. Alternatively, the particle comprises an agent that enhances function of a tissue.

In another embodiment of this aspect and all other aspects described herein, the particle is selected from a group consisting of a magnetic particle, a lipid vesicle, a microsphere, a liposome, a polymeric particle, a degradable particle, a non-degradable particle, a micelle, a nanotube, a microtubule, a quantum dot, a metal particle, a nanoshell, an inorganic particle, a lipid, a nanoparticle, a microparticle, or a dendrimer.

In another embodiment of this aspect and all other aspects described herein, the agent enhances a function selected from the group consisting of cell growth, proliferation, migration, cell differentiation, de-differentiation, aggregation, matrix production, production of trophic factors, apoptosis, homing, mobilization or engraftment.

In another embodiment of this aspect and all other aspects described herein, the agent is selected from a group consisting of a small molecule, a growth factor, a cytokine, an RNA interference molecule, a proliferation factor, a vitamin, a nutraceutical, an agent with cosmetic properties, a therapeutic agent, a diagnostic agent, a chemokine, a targeting agent, or a differentiation factor.

In another embodiment of this aspect and all other aspects described herein, the cell is used as a tissue-specific carrier for a particle comprising an agent.

In another embodiment of this aspect and all other aspects described herein, the cell is part of an aggregate, is attached to or encapsulated within a particle, or entrapped within or attached to a transplantable or injectable substrate.

In another aspect, the invention relates to an isolated and engineered cell composition comprising, a cell, and a membrane associated ligand attached to a self-assembling molecule incorporated into a surface of the cell.

In one embodiment of this aspect and all other aspects described herein, the self assembling molecule is amphiphilic.

In another embodiment of this aspect and all other aspects described herein, the membrane associated ligand is attached covalently to the self-assembling amphiphilic molecule. Alternatively, the membrane associated ligand is attached non-covalently to the self-assembling amphiphilic molecule.

In another embodiment of this aspect and all other aspects described herein, at least one particle is bound to the cell. Alternatively, 1 to 10 or more particles are bound to the cell e.g., 2, 3, 4, 5, 6, 7, 8, 9, 10, or more.

In another embodiment of this aspect and all other aspects described herein, the particle comprises a ligand that is a targeting moiety.

In another embodiment of this aspect and all other aspects described herein, the cell is part of an aggregate, attached to or encapsulated within a particle, or entrapped within or attached to a transplantable or injectable substrate.

Also described herein is a method for forming an isolated and engineered cell composition, the method comprising the steps of: (a) attaching a membrane associated ligand to a self-assembling molecule to form a modified molecule, (b) forming a vesicle with the modified molecule, and (c) fusing the vesicle with a cell.

In another embodiment of this aspect and all other aspects described herein, the isolated and engineered cell composition is formed in vivo.

In another embodiment of this aspect and all other aspects described herein, the membrane associated ligand is used to form a cell aggregate.

In another embodiment of this aspect and all other aspects described herein, the particle comprises a ligand that is used to form a cell aggregate.

Also described herein is a method for forming an isolated and engineered cell composition, the method comprising the steps of: (a) attaching a membrane associated ligand to a self-assembling molecule to form a modified molecule, (b) forming a micelle with the modified molecule, and (c) fusing the micelle with the cell.

In another embodiment of this aspect and all other aspects described herein, the particle is bound to the cell through conjugation to the self-assembling molecule prior to incorporation into a cell membrane. Alternatively, the particle is bound to the cell through conjugation to the self-assembling molecule after incorporation into a cell membrane.

Another aspect of the present invention relates to an isolated and engineered cell composition comprising a cell and a membrane associated ligand attached to the surface of the cell, wherein said membrane associated ligand is attached to a first portion of the surface of the cell, and wherein a second portion of the surface of the cell is free from the ligand.

In another embodiment of this aspect and all other aspects described herein, the cell further comprises a particle.

In another embodiment of this aspect and all other aspects described herein, the membrane associated ligand is used to form a cell aggregate.

In another embodiment of this aspect and all other aspects described herein, the aggregate is formed in vivo.

In another embodiment of this aspect and all other aspects described herein, the cell is part of an aggregate, attached to or encapsulated within a particle, or entrapped within or attached to a transplantable, or injectable substrate.

Another aspect described herein is a method of treating an individual in need of targeted tissue regeneration, the method comprising the steps of: (a) forming a targeted cell by attaching a membrane associated ligand to a surface of the cell, wherein said membrane associated ligand confers accumulation of the targeted cell in a tissue to be treated; (b) forming a dual functional cell by attaching a particle to the surface of the targeted cell, wherein the particle comprises an agent, which enhances function of the dual functional cell; and (c) administering the dual functional cell to an individual in need of targeted tissue regeneration.

In another embodiment of this aspect and all other aspects described herein, the cell is isolated from an individual in need of targeted tissue regeneration. Alternatively, the cell is isolated from an individual other than the individual in need of targeted tissue regeneration.

In another embodiment of this aspect and all other aspects described herein, the cell is expanded in an ex vivo culture environment prior to attaching the membrane associated ligand.

In another embodiment of this aspect and all other aspects described herein, the particle is released at the site of targeted tissue regeneration.

Another aspect described herein is a method for vaccinating a subject, the method comprising the steps of: (a) forming a targeted dendritic cell by attaching a membrane associated ligand to a surface of a dendritic cell, wherein the membrane associated ligand confers accumulation of the targeted dendritic cell in lymph tissue; (b) forming an activated targeted dendritic cell by contacting the targeted dendritic cell with an antigen; and (c) administering the activated targeted dendritic cell to a subject in need of a vaccine.

In another embodiment of this aspect and all other aspects described herein, the antigen comprises a viral antigen. Alternatively, the antigen comprises a bacterial antigen, or a cancer associated antigen.

Also described herein is a kit for modifying a cell, the kit comprising: (a) a self-assembling molecule with an attached moiety, (b) instructions comprising a method for modifying a cell, and (c) packaging materials therefor. The instructions included in the kit describe a method for forming an isolated and engineered cell composition comprising the steps of: (a) forming a vesicle or micelle with the self-assembling molecule with an attached moiety, and (b) fusing the vesicle or micelle with a cell of interest.

In another embodiment of this aspect and all other aspects described herein, the attached moiety is a membrane associated ligand. Alternatively, the attached moiety is a particle. In one embodiment, the kit can also include a particle in addition to a second moiety.

Also described herein is an isolated and engineered cell composition comprising (a) a cell; (b) a particle attached to a surface of the cell; and (c) a particle associated ligand not bound to the cell. In such embodiments, it is preferred that the ligand present on the particles does not interact directly with the cell, but rather that the particles contain the ligands, either internally (e.g., withing the particle), externally (e.g., on the particle surface), or incorporated into the particle (e.g., modified amphiphilic molecule that becomes a component of the cell surface). It is preferred that the ligand does not act as a linker molecule that binds the particle to the cell.

In another embodiment of this aspect and all other aspects described herein, a particle comprises an agent, and in some cases the agent is a component of the particle. In these embodiments, approximately 1% to substantially all of the particle can be composed of the agent of interest.

In another embodiment of this aspect and all other aspects described herein, the particle is 1 nm-5000 nm in size.

In another embodiment of this aspect and all other aspects described herein, the particle is not internalized into the cell.

In another embodiment of this aspect and all other aspects described herein, the particle further comprises an agent that is sensitive to inactivation by contact to plasma or serum.

In another embodiment of this aspect and all other aspects described herein, the agent sensitive to inactivation is an RNA interference molecule.

In another embodiment of this aspect and all other aspects described herein, the agent enhances trafficking of the cell.

In another embodiment of this aspect and all other aspects described herein, the agent comprises a therapeutic agent. Alternatively, the agent comprises a diagnostic agent.

In another embodiment of this aspect and all other aspects described herein, the agent enhances function of host cells and/or tissue.

Another aspect described herein is an isolated and engineered cell composition comprising: (a) a cell; and (b) a membrane associated ligand attached to a lipid molecule incorporated into a surface of the cell.

In another embodiment of this aspect and all other aspects described herein, the lipid molecule comprises a single tail. Alternatively, the lipid molecule can comprise multiple tails.

In another embodiment of this aspect and all other aspects described herein, the lipid molecule comprises multiple charges.

In another embodiment of this aspect and all other aspects described herein, the lipid molecule is an engineered lipid such as a lipidoid.

Another aspect described herein relates to a method for preparing a cell composition in which a portion of the ligand (e.g., at least one-third of the ligands) are stable on the cell surface for at least 2 days after modification, the method comprising: (a) preparing lipid vesicles comprising a source of biotin and a ligand, (b) contacting a cell (e.g., human mesenchymal stem cell) with said vesicles, wherein a cell composition is formed such that the ligand is present on the cell for at least 2 days after said contacting step.

In one embodiment of this aspect and all other aspects described herein, the ligand comprises biotin.

Also described herein is a method for modifying a progenitor or stem cell with a ligand without compromising stem cell characteristics, the method comprising the steps of covalently modifying the cell surface, wherein the cell composition is formed without loss of progenitor or stem cell characteristics.

Also described herein is a method for modifying a progenitor stem cell with a ligand without compromising stem cell characteristics, the method comprising the steps of: (a) contacting a cell with a source of biotin, (b) contacting said cell of step (a) with streptavidin and a ligand, wherein a stem cell composition is formed without loss of stem cell characteristics.

In one embodiment of this aspect and all other aspects described herein, the stem cell characteristics comprise multilineage differentiation, viability, proliferation, secretion of paracrine factors, transendothelial migration in vivo, and/or adhesion.

In one embodiment of this aspect and all other aspects described herein, the ligand comprises Sialyl Lewis X.

Also described herein is a method for attaching an adhesion ligand to the surface of a cell, the method comprising the steps of: (a) contacting a cell with a source of biotin, (b) contacting said cell of step (a) with streptavidin and an adhesion ligand that promotes firm adhesion, wherein a cell composition is formed comprising an adhesion ligand on the cell surface and wherein said adhesion ligand permits firm adhesion.

In one embodiment of this aspect and all other aspects described herein, a plurality of adhesion ligands are attached to the surface of a cell.

In another embodiment of this aspect and all other aspects described herein, the adhesion ligand comprises PSGL-1 or P-selectin antibody.

In another embodiment of this aspect and all other aspects described herein, the adhesion ligand permits rolling at a rate under 2 μm/sec up to 1.9 dynes/cm².

In another embodiment of this aspect and all other aspects described herein, the adhesion ligand enhances localization within a tissue.

In another embodiment of this aspect and all other aspects described herein, wherein the tissue is bone marrow.

In another embodiment of this aspect and all other aspects described herein, the ligands comprise specific physical functionalities (negative charge, positive charge, lipid, antibody etc.) or chemical functionalities (NHS group, streptavidin etc).

Also described herein is a method for attaching a particle to the surface of a cell, the method comprising: (a) attaching a ligand to a particle to prepare a functionalized particle, and (b) contacting said functionalized particle with a cell, wherein said functionalized particle is attached to the surface of said cell.

In another embodiment of this aspect and all other aspects described herein, the functionalized particle comprises PLGA.

In another embodiment of this aspect and all other aspects described herein, the functionalized particle is prepared using an emulsion method.

In another embodiment of this aspect and all other aspects described herein, the ligand permits localization of said cell to a tissue.

In another embodiment of this aspect and all other aspects described herein, the functionalized particle is internalized.

In another embodiment of this aspect and all other aspects described herein, the functionalized particle is not internalized.

A method in which at least one-third of the ligands attached to the lipid (which is conjugated to the cell surface) is stable after 2 days of modification, the method comprising the steps of: (a) preparation of vesicles from 1,2-Dioleoyl-sn-Glycero-3-Phosphoethanolamine-N-(Biotinyl) sodium salt, and (b) subsequent incubation of the vesicles with the hMSCs for 30 minutes, wherein the ligand stabilized on hMSCs is biotin

Also described herein is a method for attaching an adhesion ligand that promotes firm adhesion, the method comprising: (a) Modification (Biotinylation) of cell with N-hydroxy-succinimide group of Biotinyl-N-hydroxy-succinimide, (b) treatment with streptavidin and an adhesion ligand.

In one embodiment of this aspect and all other aspects described herein, the ligand comprises P-selectin antibody.

In another embodiment of this aspect and all other aspects described herein, the rolling is under 2 μm/sec up to 1.9 dynes/cm².

In another embodiment of this aspect and all other aspects described herein, the rolling enhances localization within a tissue.

In another embodiment of this aspect and all other aspects described herein, the tissue is bone marrow.

In another embodiment of this aspect and all other aspects described herein, the particle remains attached on the surface of the cell.

In another embodiment of this aspect and all other aspects described herein, the particle gets internalized within the cell after being stabilized on the cell surface.

In another embodiment of this aspect and all other aspects described herein, the surface bound particles are carried by the cells to the specific sites after systemic injection or local administration.

In another embodiment of this aspect and all other aspects described herein, the particle remains attached to the surface of the cell, or separate from the cell surface, or is internalized at the targeted site.

In another embodiment of this aspect and all other aspects described herein, the internalized particles are carried by the cells to the specific sites after systemic injection or local administration.

### BRIEF DESCRIPTION OF THE FIGURES

FIG. 1 is a schematic diagram showing cell membrane modifications: 1. Functionalization of the cell membrane to introduce a specific cell surface functional group 2. Attachment of a particle that contains a therapeutic agent to the functionalized group through a linker where the particle contains a second functional group 3. Attachment of a particle to the functionalized group on the cell surface through a linker 4. Attachment of a particle to the functionalized group on the cell surface. 5. A particle attached to a cell through the functionalized group on the cell surface where the particle and the cell contains functional ligands 6. Functionalization of cell membrane with a multifunctional agent 7. Functionalization of cell membrane through attachment of lipid molecules or lipid based vesicles 8. Attachment of functionalized particle to the cell (without functionalizing the cell membrane) either through physical (non-covalent) interactions or through chemical interactions (covalent) 9. Attachment of functionalized particle to the cell (without attaching free functional groups to the cell membrane) through a linker 10. Attachment of functionalized particle to the cell (without functionalizing the cell membrane with free functional groups) through a linker where the particle contains a ligand

FIG. 2 is a bar graph depicting the percentage of human mesenchymal stem cell (hMSCs) with particles attached at 4, 8 and 12 hours as observed by fluorescent microscopic images.

FIGS. 3A-3B are bar graphs showing internalization of particles with different surface characteristics and with different particle size with respect to time; FIG. 3A shows percentage of particles with different surface characteristics internalized by hMSC at 4, 8 and 12 hours as observed in Z-stack confocal images from y-z, x-z and x-y planes; for confocal microscopy hMSCs were stained with propidium iodide and particles are DiD encapsulated. FIG. 3B shows the effect of particle size on the internalization of the CD90 antibody coated PLGA particle attached to hMSC at 4, 8 and 12 hours showing particles lower than 3 μm are internalized with greater efficiency compared to larger particles.

FIGS. 4A-4D are a series of graphs showing a detailed analysis of negatively charged PLGA particles conjugated to hMSC; FIG. 4A shows the percentage of cells conjugated to particles at 0 hour and 36 hour (left); FIG. 4B shows the percentage of cells having 1 particle, 2 particles and 3 or more particles at 0 hour and 36 hour. This percentage is based on the total number of cells having particles. FIG. 4C shows the effect of trypsinization on the attachment of negatively charged PLGA particle to hMSC. Percentage of cells associated with particles immediately after trypsinization and subsequently on day 2 and 4 and the total number of cells in the field of view; FIG. 4D shows the percentage of cells having 1 particle, 2 particles and 3 or more particles at 0 day (day of trypsinization), 2 day and 4 day. This percentage is based on the total number of cells that contain conjugated particles.

FIGS. 5A-5D are a series of graphs depicting cell and particle characteristics of an exemplary modified cell. FIG. 5A shows the number of particles attached to hMSCs through biotin and streptavidin interaction immediately after conjugation showing that biotinylated hMSCs specifically binds to streptavidin coated particles; FIG. 5B depicts viability of PLGA attached hMSC (through biotin-streptavidin) at 0 hours and after 48 hours; FIG. 5C shows the percentage of PLGA attached hMSC (through biotin-streptavidin) adhered on tissue culture surface at 10, 30 and 90 minutes; FIG. 5D shows proliferation of PLGA attached hMSC (through biotin-streptavidin) over 8 day period.

FIG. 6 is a bar graph depicting rolling of biotinylated cells modified with Strep-PLGA particles where the cells and particles are functionalized with SLeX on P-selectin; the rolling response of hMSCs attached with PLGA particle through biotin-streptavidin and biotinylated Sialyl Lewis X (SLeX) on P-selectin coated substrate at 0.36 dynes/cm² in a parallel plate flow chamber assay is shown; the Control group includes unmodified i.e. PBS treated hMSCs (Rolling assays were performed both in brightfield and fluorescent modes).

FIG. 7 is a bar graph depicting conjugation of PLGA particles to cell surface (with a PEG linker and without functionalizing the cell; the average number of PLGA particle with N-hydroxy succinimide (NHS) group, with and without a polyethylene glycol (PEG) linker attached to hMSC (C1, C2 and C3 represents the concentration of PEG linker used to functionalize the NHS activated PLGA particle) are shown, indicating that a greater number of particles are conjugated to cells when the particles are functionalized with a higher concentration of PEG linker.

FIGS. 8A and 8B are bar graphs depicting localization of modified and unmodified mesenchymal stem cells (MSCs) in bone marrow; FIG. 8A shows the number of unmodified and modified MSCs (MSCs are modified with biotin-N-hydroxy succinimde followed by streptavidin and biotinylated sialyl Lewis X, SLeX) localized in the bone marrow after 2 hours of tail vein injection of cells in three separate experiments. Compared to unmodified MSCs, MSCs with conjugated SLeX are localized to bone marrow with increased numbers. FIG. 8B shows the number of unmodified and unmodified MSCs extravasated from bone marrow endothelium after 24 hours of tail vein injection of cells.

FIG. 9 is a series of bar graphs showing secretion of paracrine factors SDF-1, IGF-1 and PGE2 in cell culture supernatant by PBS treated cells and SLeX modified MSCs at 24 hour at 37° C.

FIG. 10 is a series of graphs showing adhesive interactions of modified and unmodified MSCs on P-selectin surfaces under flow conditions in a flow chamber assay; FIG. 10A shows unmodified MSCs on P-selectin surface do not exhibit adhesive interaction with the surface under flow conditions and the velocity of the cells are 70 μm/sec at shear stress 0.36 dyne/cm² indicating that without any modification the MSCs do not interact specifically with P-selectin; FIG. 10B shows that more than 80% of MSCs modified with Ab-P selectin (antibody of P-selectin) interact with the substrate up to a shear stress of 10 dyne/cm² either through firm adhesion or through rolling with a velocity less than 3 μm/sec up to 10 dyne/cm². This indicates that conjugation of adhesion ligands (in this case antibody of P-selectin) induces adhesive interaction of cells with P-selectin under shear conditions.

FIG. 11 is a bar graph depicting quantification of biotin ligands on the surface of the hMSCS biotinylated with biotin-N-hydroxysuccinimide in adherent and suspension mode and measured by Biotin-HABA-Avidin assay.

FIG. 12 is a bar graph depicting fluorescence intensity (FI) obtained from the cells that were decorated with dye (DiD) encapsulated self-assembled fibers in an adherent method. Fibers were generated through self-assembly of amphiphiles, during self-assembly process fluorescent dye (DiD) has been encapsulated. This was done in adherent mode, wherein half of the cell surface has been modified.

FIG. 13 is a bar graph showing fluorescence intensity (FI) obtained from the cells that were decorated with dye (DiD) encapsulated self-assembled fibers in suspension method. Fibers were generated through self-assembly of amphiphiles, during self-assembly process fluorescent dye (DiD) has been encapsulated. This was done in “suspension mode”, wherein the full surface of the cell has been modified.

FIGS. 14A-14B are a series of bar graphs showing fluorescence intensity of modified cells; FIG. 14A shows modification of hMSC by vesicles in an adherent mode. Biotinylated lipid vesicles added to hMSCs followed by rhodamine conjugated streptavidin. Modification of the hMSCs was measured as a function of fluorescent signal of rhodamine conjugated streptavidin immediately after conjugation and at 7 day. The fluorescence of vesicle modified hMSCs shows that through these methods biotinylated lipids have been incorporated onto the cells and they are associated with the cell until the 7th day. FIG. 14B shows stability and accessibility of biotin (conjugated to hMSCs by biotinylated lipid vesicles) on hMSCs surface as measured by the fluorescent signal of rhodamine conjugated streptavidin added to biotin lipid vesicles conjugated to MSCs over a 7 day period where MSCs were biotinylated on day 0 and the fluorescent signal of rhodamine-streptavidin was measured by addition of rhodamine-streptavidin on day 0, 2, 4 and 7. Fluorescence intensity shows that at least one third of biotin on the cell surface is accessible for modification up to second day, and around one fourth of the biotins are still accessible on the surface for further modification.

FIGS. 15A-B show the rolling interaction of the vesicle modified hMSCs on a P-selectin coated substrate in a flow chamber assay. FIG. 15A shows velocity of SLeX attached hMSCs through unmodified and vesicles modified method on P-selectin treated surfaces at the shear stress 0.5 dyne/cm². FIG. 15B shows the effect of shear stress on the rolling velocities of vesicles-modified hMSCs.

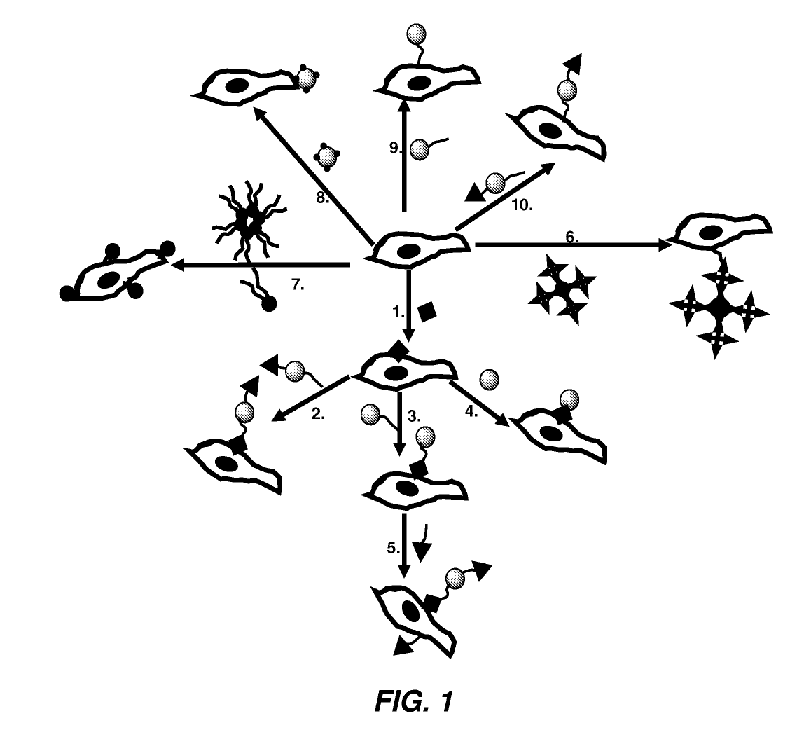

### DETAILED DESCRIPTION

The present invention is directed towards methods for functionalizing the cell surface by various techniques and for use in a range of applications. The compositions and methods described herein are useful for targeting cells to tissues for regeneration, or alternatively for the cell-based delivery of agents to a particular tissue of interest. Cells useful for the methods described herein can be modified by the addition of a particle, as well as a ligand. The particle may further comprise an agent, such as a protein, a small molecule, an RNA interference molecule, a drug, a vitamin, a therapeutic agent, a diagnostic agent a nutraceutical, an agent that has cosmetic properties, or a nucleic acid. In addition, the compositions described herein can be tailored by one of skill in the art for the treatment of a wide range of diseases or wounds, including for example, stroke, organ regeneration, cancer, fractures, and ischemic heart disease, among others.

Cells and their inner machinery are an indispensible component of many biological applications, whether they are added exogenously or targeted within the body. Microparticle and nanoparticle based targeted delivery to specific cells and sites have attained considerable attention. Targeting of cells through particle based approaches using the methods of the present invention by utilizing cell specific interactions is useful for delivering drugs and other factors to specific tissues. We have shown that functionalized particles can be targeted to the cells locally or through systemic administration.

The methods and compositions described herein include a variety of approaches to functionalize the cell membrane to direct cell function or control microenvironmental signals through either mimicking or engineering alternative interactions between the cell membrane and the extracellular matrix. This is accomplished by engineering the cell by attaching particles and/or polymeric chains with specific functionalization. Functionalization of the cell can be achieved by chemically attaching particles, or by directly functionalizing the membrane with non-particle coatings. This method can be expanded to include genetic functionalization of the cell through delivery of a binding and/or soluble agent to change the genetic (and/or protein) expression of the cells. This can be achieved, for example by viral and non-viral gene therapy, siRNA delivery, among others.

Applications of the methods and compositions of the present invention include, but are not limited to: directed cell migration/invasion under physiological and pathological states, targeted and/or controlled release of bioactive agents (growth factor, enzymes etc.) within extracellular matrices or via systemic targeting, cell sensor applications to detect changes in cellular phenotype (e.g., receptor expression, tracking and/or imaging of cells).

As used herein, the terms “isolated” and “isolating” are used to describe the process of segregating a selected cell type from a biological sample from a mammal. Methods for cell isolation are well known to those of skill in the art, and generally involve an enzymatic reaction (e.g., collagenase to dissociate cells from a desired tissue or biological sample (e.g., blood)), centrifugation, and/or plating of cells in tissue culture dishes. Methods suitable for the isolating cells for the methods and compositions described herein can be found in, for example U.S. Pat. No. 6,475,764; 5,424,208; 7,217,568; 6,991,897; or 6,627,759, which are incorporated herein by reference in their entirety. It is specifically contemplated herein that a homogeneous population of cells or a heterogeneous (e.g., a plurality of cell types) population of cells can be used for the practice of the methods and compositions described herein.

As used herein, the terms “functionalization”, or “functionalized” are used to describe modifications to a cell membrane, which permits the cell to have a desired function, for example targeting to a tissue to be treated, or drug delivery. Functionalization of a cell can encompass, for example attaching a polymer, a linker, a particle, a targeting moiety, a chemical side group, a ligand, or any combination of these. Functionalization also encompasses attaching, for example a particle to a linker molecule on the surface of the cell. In addition, the cell can be functionalized by attaching a moiety to a cell membrane or by loading an agent into the cell for drug delivery. If so desired, a cell can also be functionalized for two different purposes e.g., targeting to a tissue and receptor-mediated uptake, or targeting to a tissue and drug delivery. Such cells are referred to herein as a “dual functional” cell. In some cases, the functionalization of a cell can occur in vivo by injecting, for example a vesicle formed from self-assembling amphiphilic molecules into a tissue in order to engineer cell surfaces (e.g., the cell surfaces can be modified in situ or in vivo).

As used herein, the terms “ligand” or “membrane associated ligand” are used to describe an exogenous moiety attached to the cell membrane that has a biological action or potential for such action, such as binding to a receptor, a cell surface polypeptide, a membrane or a carbohydrate, among others. By “exogenous” is meant that the ligand is not synthesized within the organism or system. In some cases, a ligand can act as a targeting moiety by permitting cells to be directed toward a particular organ or tissue for targeted tissue regeneration (e.g., repair of a damaged tissue). Targeting moieties can include, for example, a drug, a receptor, an antibody, an antibody fragment, an aptamer, a peptide, a vitamin, a carbohydrate, a protein, an adhesion molecule, a glycoprotein, a sugar residue or a glycosaminoglycan, a therapeutic agent, a drug, or a combination of these. A skilled artisan can readily design various targeting moieties for modifying a cell based upon an intended purpose for that cell.

As used herein, the term “particle” is used to describe a moiety that is attached to a cell membrane, that can be used to deliver an agent, or a mixture of agents, or to provide functionality to a cell. The term “particle” encompasses, for example a magnetic particle, a lipid vesicle, a microsphere, a liposome, a polymeric particle, a degradable particle, a non-degradable particle, a micelle, a nanotube, a microtubule, a quantum dot, a metal particle, a nano-shell, an inorganic particle, a nanoparticle, a microparticle, a lipid, or a dendrimer. Particle size can vary widely from approximately 0.1-10,000 nm in size, however preferably a particle is approximately 1-8000 nm in size. Particles are considered nanoparticles when they are approximately 1-999 nm in size, or microparticles when they are approximately 1000 nm-8000 nm in size. A skilled artisan can readily design various particles to attach to a cell based upon an intended purpose for that particle.

As used herein, the term “agent” is used to describe a bioactive molecule or precursor to a bioactive molecule that can induce a cell or tissue effect, or alternatively a cell composition that is delivered. An agent can be a small molecule, a drug, a growth factor, a cytokine, an enzyme, an RNA interference molecule (e.g., siRNA, shRNA, or miRNA), a proliferation factor, a prodrug, a zymogen, a vitamin, a nutraceutical, a therapeutic agent, a diagnostic agent, a chemokine, a de-differentiation factor, or a differentiation factor. Functions that can be modulated by administration of an agent include, for example cell growth, proliferation, migration, cell differentiation, de-differentiation, aggregation, matrix production, production of trophic factors, apoptosis, homing, mobilization, or engraftment. For example, if one wishes to target bone tissue for tissue regeneration, one could treat cells with an agent (or a plurality of agents) to induce differentiation into an osteogenic lineage as is described herein in the Examples section. Such agents include, but are not limited to, dexamethasone, (3-glycerophosphate, and L-ascorbic acid-2-phosphate.

As used herein, the term “targeted tissue regeneration” is used to describe treatment of an organ or a tissue for relief of damage or disease. “Relief of damage of disease” can be measured by a reduction in severity of a disease, a reduction in symptoms, a complete remission of the disease, or a change in any other measurable parameter associated with the disease as known to one skilled in the art of medicine. Targeted tissue regeneration encompasses delivery of an agent (or a plurality of agents) to a damaged or diseased tissue, as well as administering cells to re-populate a damaged or diseased tissue. Diseases or disorders that can be treated in this manner include, for example ALS, Crohn's disease, spinal cord injuries, cardiac disease, stroke, autism, lupus, eye diseases, multiple sclerosis, chronic obstructive pulmonary disease, arthritis, diabetes, autoimmune disorders, ischemic heart disease, cancer, wound healing, burns, and Parkinson's disease. Essentially any tissue or organ can be targeted for treatment with the methods and compositions disclosed herein, which include the brain, heart, vascular system, pulmonary system, renal system, splenic system, lymphatic system, bone marrow, bone, skeletomuscular system, immune system, reproductive system, skin, cartilage, nervous system, gastrointestinal system, liver, pancreatic system, hematopoietic system, a hormonal system, among others.

As used herein, the term “vesicle” refers to a spherical lipid structure comprising an amphiphilic bilayer, and can further comprise a bioactive agent. Such spherical structures are also referred to herein as “liposomes”. In contrast, the term “micelle” refers to a spherical lipid structure comprising an energetically favorable conformation for a monolayer of amphiphilic molecules (e.g., phospholipids). In general, a micelle comprises an outer hydrophilic sphere and an inner hydrophobic region. Delivery of bioactive agents using a micelle is also contemplated herein. As used herein, the term “amphiphilic molecule” refers to a molecule that comprises a hydrophilic region on one end, and a hydrophobic region on the opposite end (e.g., a phospholipid). The term “amphiphilic molecule” also encompasses the term “lipid molecule”, as used herein. The term “lipidoid” refers to a nanoparticle formulation for the systemic delivery of an RNA interference molecule and is described in Akinc et al., Nature Biotechnology advance online publication, 27 Apr. 2008 (DOI:10.1038/nbt1402).

An “RNA interference molecule” as used herein, is defined as any agent which interferes with or inhibits expression of a target gene or genomic sequence by RNA interference (RNAi). Such RNA interfering agents include, but are not limited to, nucleic acid molecules including RNA molecules which are homologous to a target gene or genomic sequence, or a fragment thereof, short interfering RNA (siRNA), short hairpin or small hairpin RNA (shRNA), microRNA (miRNA) and small molecules which interfere with or inhibit expression of a target gene by RNA interference (RNAi).

As used herein, the term “source of biotin” refers to a compound comprising a biotin moiety that permits biotinylation of a cell or particle surface. Sources of biotin are well known in the art. In one embodiment, the source of biotin is 1,2-Dioleoyl-sn-Glyerco-3-Phosphoethanolamine-N-(Biotinyl) sodium salt. In another embodiment the source of biotin comprises Biotinyl-N-hydroxy-succinimide.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

### Advantages and Uses

One approach that has been used to modify the cell surface includes biotinylation of sialic acid residues present on the cell membrane²⁶. This approach was used to pattern cells on defined substrates. Similarly, a sialic acid residue was used to chemically attach a biotinylated phosphine based linker²⁷. Progenitor cells have been targeted to particular regions in the body through ‘painting’ cell membranes with antibodies to matrix molecules for promoting the adherence of stem or progenitor cells to a cartilage injury site²⁸. This strategy relied on a two-step process wherein lipidated protein G (with a hydrophobic group, palmitic acid²⁸) was first allowed to intercalate into cell membranes, and a second incubation in a solution of antibodies to cartilage matrix antigens allowed the binding of the antibodies to protein G on the external surface of the cell. Other examples of cell surface engineering include: unnatural N-acyl substituents for sialic acid of glycans²⁹, reaction of ketone group of acetamidosugars³⁰, derivatization of sialic acid by thiol groups³¹, periodate oxidation of sialic acid³², and chemical modification of intracellular proteins (e.g., AGT)³³. Electro active modification of a cell surface with RGD is an example of the use of external electrical field to modify the cell surface³⁴. These results show that a surface of a cell membrane can be chemically and/or physically modified. The methods and compositions described herein are further extended to engineer the cell surface by a particle and/or membrane based technology to functionalize the cell surface as a platform technology for influencing cellular interactions with its microenvironment.

The use of a surface bound magnetic nanoparticle for cell isolation has been shown^{35,36}, and magnetic nanoparticles are also used for cell tracking³⁷. These data indicate that larger nanoparticles (e.g., as large as 0.9-1.0 μm) can be taken up without any adverse effect on hematopoietic stem cells and allows detection at lower resolutions. These beads are typically pre-coated with secondary antibodies against primary antibodies from various species or biotin, allowing one to easily construct a system to isolate cells using a primary antibody of choice. Once the beads have captured the cells, tubes containing the beads are placed in a Magnetic Particle Concentrator. Bound cells are quickly pulled to the tube wall and the supernatant can be transferred to a new tube or discarded, depending on the chosen method. Certain beads also contain a cleavable site to permit facile release of particles from the cell surface. Studies with Dynabeads show higher bead concentrations lead to increased numbers of beads on the cell surface but may result in toxicity³⁶. Moreover magnetic nanoparticles³⁸ can be easily modified with, for example peptides and/or hydrophilic polymers (PEG) to prevent internalization³⁹. Studies to track the receptor molecule behavior on the cell surface shows that binding of labeled PE-IgG particles can be used to monitor the mobility and the dynamic associations of the receptors⁴⁰.

Functionalization of cells with polymers or particles, as described herein, offers numerous advantages for biomedical applications including control over cell function and control over the cellular microenvironment.

In contrast to targeting the cells with free flowing functionalized particles and/or polymers⁴¹, this method allows the creation of a functionalized cell for a particular application. Moreover, compared to targeted delivery systems (where the particles are targeted to release the agent to the specific cells)⁴², this method would allow better control as the cells are functionalized to perform the desired actions. The different combination of properties that can be achieved by these types of functionalizations would allow the cells to perform multiple tasks and direct the cells to perform those tasks in a controlled and desired fashion.

The use of functionalized cells has various applications. For example, one can target delivery of cells to specific sites. The functionalized cells can be precisely targeted toward the site that requires a cell for regeneration or for delivery of an agent e.g. delivery of different vector at a particular tumor site. Also, one can functionalize cells by programming them to migrate with specific directionality or achieve greater invasive potential within 3D matrices. For example, the functionalized cells are able to degrade the extracellular polymer matrix through the functional particle attached on the cell surface and therefore the migration rate is increased. This method may be useful for achieving cell distribution within a tissue, for example a tumor, and for delivery of particular agents. One can also use the functionalized cell to deliver an agent or a plurality of agents, i.e. a drug, a growth factor or an enzyme for the purpose of a particular application (e.g. tissue regeneration, cancer treatment to kill specific cells etc.). The cells may also be genetically modified to produce specific agents and thus functionalization may be used to direct the cell to deliver these agents within particular environments. The delivery of agents directly to cells may reduce the need for repeated dosing. One can further increase the viability of the cells by preventing its destruction by phagocytosis (e.g. macrophages). The cells can be used for controlled release of agents to cells for extended periods, for example an RNA interference molecule (e.g., siRNA, shRNA, miRNA). This will significantly reduce the quantity of siRNA required for treatment as the drug can be directed to the cell without substantial interaction with the surrounding microenvironment (i.e. avoid degradation). Agents can be directed away from cells to modify the microenvironment without initially and/or directly affecting the cells. Particles can serve to adhere cells to particular tissues (cell immobilization) or other cells within the body or within in vitro model systems.

The applications of the present invention are vast and are applicable to a variety of disease states. Some non-limiting examples of applications and disease states include the following: targeting of immune cells (e.g., T-cells), osteoporosis (prevention and treatment), Osteogenesis imperfecta, inflammatory diseases (e.g., Chrohn's disease, graft versus host rejection, arthritis, celiac disease etc), aging (e.g., decreased degeneration and increased regeneration), ischemic tissue (e.g., myocardial infarction and related disorders, and general muscle degeneration), cardiovascular disease (e.g., peripheral artery disease), cancer, acute radiation syndrome, lung disease, heart disease, diabetes, liver and kidney failure, stroke, baldness, wound healing, brain disease/damage, and nerve disease or damage. It is well within the ability of one skilled in the art to apply the methods and compositions described herein for the therapy of a disease.

### Cells

Essentially any cell can be used in the methods and compositions described herein. For animal use it is preferred that the cell is of animal origin, while for human use it is preferred that the cell is a human cell; in each case an autologous cell source is preferred. The cell can be a primary cell e.g., a primary hepatocyte, a primary neuronal cell, a primary myoblast, a primary mesenchymal stem cell, primary progenitor cell, or it may be a cell of an established cell line. It is not necessary that the cell be capable of undergoing cell division; a terminally differentiated cell can be used in the methods described herein. In this context, the cell can be of any cell type including, but not limited to, epithelial, endothelial, neuronal, adipose, cardiac, skeletal muscle, fibroblast, immune cells, hepatic, splenic, lung, circulating blood cells, reproductive cells, gastrointestinal, renal, bone marrow, and pancreatic cells. The cell can be a cell line, a stem cell, or a primary cell isolated from any tissue including, but not limited to brain, liver, lung, gut, stomach, fat, muscle, testes, uterus, ovary, skin, spleen, endocrine organ and bone, etc.

Where the cell is maintained under in vitro conditions, conventional tissue culture conditions and methods can be used, and are known to those of skill in the art. Isolation and culture methods for various cells are well within the knowledge of one skilled in the art.

If so desired, a cell can be treated prior to functionalization with a ligand and/or a particle. Cells can be pre-treated with various agents to promote expression of certain receptors on the cell surface, or to promote the cell to produce specific factors in order to enhance its homing and engraftment, or alternatively to promote a specific cell function prior to systemic delivery. For example, a cell can be induced to have enhanced cell migration prior to delivery to a subject for treatment.

In addition, both heterogeneous and homogeneous cell populations are contemplated for use with the methods and compositions described herein. In addition, aggregates of cells, cells attached to or encapsulated within particles, cells within injectable delivery vehicles such as hydrogels, and cells attached to transplantable substrates including scaffolds are contemplated for use with the methods and compositions described herein.

### Ligands

Methods for attaching a ligand to the surface of a cell composition are described herein. A ligand is a moiety attached to a cell surface, which permits the cell to have a desired biological interaction with a tissue in vivo. A ligand can be an antibody, an antibody fragment, an aptamer, a peptide, a vitamin, a carbohydrate, a protein, a receptor, an adhesion molecule, a glycoprotein, a sugar residue, a therapeutic agent, a drug, a glycosaminoglycan, or any combination thereof. For example, a ligand can be an antibody that recognizes a cancer-cell specific antigen and thus the cell interacts preferentially with tumor cells to permit tumor-specific delivery of an agent. In general, for the methods and compositions described herein, the ligand (or plurality of ligands) are exogenous (i.e., not synthesized within the organism or system). A ligand can confer the ability of a cell composition to accumulate in a tissue to be treated, since a preferred ligand is capable of interacting with a target molecule on the external face of a tissue to be treated. Ligands having limited cross-reactivity to other tissues are generally preferred. A stealth ligand is a ligand that is not exposed (e.g., is encapsulated, or entrapped within a particle) until it reaches the tissue of interest. An advantage of using a stealth ligand is to limit any non-specific effects or side effects that can occur when treating systemically with a ligand (e.g., a drug).

In some cases a ligand can act as a targeting moiety, which permits the cell to target to a specific tissue. Such suitable targeting moieties can include, for example, any member of a specific binding pair, antibodies, monoclonal antibodies, or derivatives or analogs thereof, including without limitation: Fv fragments, single chain Fv (scFv) fragments, Fab′ fragments, F(ab′)₂ fragments, single domain antibodies, camelized antibodies and antibody fragments, humanized antibodies and antibody fragments, and multivalent versions of the foregoing; multivalent binding reagents including without limitation: monospecific or bispecific antibodies, such as disulfide stabilized Fv fragments, scFv tandems ((scFv)₂ fragments), diabodies, tribodies or tetrabodies, which typically are covalently linked or otherwise stabilized (i.e., leucine zipper or helix stabilized) scFv fragments; and other targeting moieties include for example, aptamers, receptors, ligands, and fusion proteins.

A skilled artisan can select any targeting moiety based on the purpose of the cell, for example an estrogen receptor ligand, such as tamoxifen, can target cells to estrogen-dependent breast cancer cells that have an increased number of estrogen receptors on the cell surface. Other non-limiting examples of ligand/receptor interactions include CCR1 (e.g., for treatment of inflamed joint tissues or brain in rheumatoid arthritis, and/or multiple sclerosis), CCR7, CCR8 (e.g., targeting to lymph node tissue), CCR6, CCR9, CCR10 (e.g., to target to intestinal tissue), CCR4, CCR10 (e.g., for targeting to skin), CXCR4 (e.g., for general enhanced transmigration), HCELL (e.g., for treatment of inflammation and inflammatory disorders, bone marrow), Alpha4beta7 (e.g., for intestinal mucosa targeting), VLA-4/VCAM-1 (e.g., targeting to endothelium). In general, any receptor involved in targeting (e.g., cancer metastasis) can be harnessed for use in the methods and compositions described herein. In some embodiments, the modifying ligand comprises a stealth ligand, such as poly(ethylene glycol), hyaluronic acid, dextran, chitosin, or poly(ethylene oxide).

### Particles

The properties of the particles attached to the cell can differ between types of particles or can even differ within a single particle, for example with respect to a number of parameters including their size, morphology, composition, surface charge, porosity, surface texture, concentration of functional domains or type of domain, degradation profile, whether they contain one or more agents (including growth factors, magnets, cytokines, adhesive agents, toxins, proteins, peptides, enzymes, nucleic acid, antibodies, cell receptors, or fragments thereof), the location of such agent (e.g., on the surface or internally), etc. If so desired, the particle can be composed of an agent, such that approximately 1% to substantially the entire particle (i.e., approximately 100%) is the desired agent. In addition to containing a potential magnetic agent, the particle may be a coated or uncoated magnet.

Particles can be attached to a cell surface directly through a direct interaction with the cell membrane. The functionality present on the particle can be polymeric, non-polymeric or oligomeric. The binding sites on the particle can be ionic (both cationic and anionic) or non-ionic provided that the particle can interact with the cell surface. Attaching a particle to a cell approach can be performed using a ‘bottom-up’ approach where the cell surface is pre-functionalized by various chemical and/or physical methods. The functionalized cell surface can then be used to attach the polymeric particles to fabricate the functionalized cells.

The size and shape of the particles are important in determining the fate of the particles in targeting. Generally, particles >200 nm are internalized by cells and are therefore adsorbed on the cell membrane²². Recent studies show that rod shaped particles are not as effectively internalized compared to spherical particles²³. The properties of the material also has a great impact on internalization²². For example, hydrophobic (e.g. polystyrene particles) and less adhesive (e.g. PVA) particle coatings may be used to inhibit cellular uptake²⁴. Moreover, the surface charge of the polymer particles is also important in determining the internalization fate of the particles²⁵. These studies indicate that it is likely that cell surfaces can be modified functionally without the internalization of the particles, however a method for functionalizing cells in this manner has not been described.

To avoid the possibility of internalization of the particles, the dimension of particles can be increased by introducing spacer molecules in between the particle and the cell surface. This will reduce internalization as observed in tumor cells, which show a decreased uptake of PEGylated nanoparticles compared to non-tumor cells. Similarly, particles can be attached to the surface of a cell with a functionalized spacer (or linker) molecule. A linker is a molecule that is capable of being attached to the particles directly or indirectly, via any physicochemical interaction and is further described herein in the detailed description. A particle and binding agent are “linked directly” if they are covalently or non-covalently bound to one another with no intervening structures. The particle and binding agent are said to be “linked indirectly” if they are connected to one another via a linker.

Another ‘bottom-up’ approach can be used wherein the cell surface is functionalized first, followed by the attachment of the linker and the functionalized particle. The choice of linker molecule would be such that one end adheres to a pre-functionalized cell and the other end attaches to a functionalized particle. The binding agent (on the pre-functionalized cell) or the linker is conjugated to a functional group on the particle. Alternatively, the particle or the linker is conjugated to a functional group of the binding agent.

Another particle based approach is achieved using heterogeneous (e.g. janus) particles with different features. One half of the particle can have cell adherent functionalities, which would allow the particle to interact with the cell surface (i.e. cationic polymers have been shown to preferentially interact with the cell membrane) while the other half of the particle would be designed for the desired application of the methods described herein, for example drug delivery.

Particle properties may differ from one another (e.g., a heterogeneous population) or may differ within a single particle population with respect to many parameters including, but not limited to, size, diameter, shape, composition, surface charge, degradation profile, whether they contain one or more agents, or the location of such agent (e.g., on the surface or internally).

One of the modifications of the ‘heterogeneous particles’ includes targeting one half the particle (which is bound to the cells) to deliver an agent and the other half is functionalized to perform a specific function including, but not limited to, applications such as directed cell migration, directed cell attachment and targeted delivery among others.

Another type of functionalization can be achieved by using material that contains two different functionalities separated by a linker molecule. One of the two functionalities specifically interacts with the cell (preferentially within the cell membrane or cytoplasm) whereas the other functionality is present in the external environment for the desired application of the methods described, for example drug delivery. The other functionality attached to the cell can be internalized through the cell membrane and can act as a sensor and/or marker for the cell or can be bound to the surface of the cell membrane by different approaches.

Another technique involves assembling polymer chains to coat the cell surface through proper interaction between the polymer chain and the cell membrane.

Functionalized (e.g. NHS, peptides, epoxy, imidoester, etc.) polymers can be used to encase the cell membrane so that the functionalized polymer interacts with the cell surface. This technique can be applied by sequential adsorptions of polymers or by emulsion techniques known to those of skill in the art.

Polymers can be sequentially applied to the cell membrane such that the polymer forms a layer over the cell membrane. In addition, functionalized particles can be adhered to the polymer layer for the desired application by e.g., sequential adsorption or by attachment of a functionalized particle to pre-adsorbed polymer on a cell surface.

Both of these techniques would allow the cell membrane to be functionalized in a more efficient manner compared to a particle based approach. The membrane based approach utilizes the same concept of modifying the cell surface by binding the polymer through the proper binding sites. The binding interaction can be physical e.g. ionic in charged polymer, antibody-antigen interaction etc. Similarly the interaction can be chemical depending on the polymer functionality (amine, carboxyl, sulphide, etc.).

Particles and/or linkers at the site of conjugation may also contain a cleavable site that is cleaved in response to a biological event or controlled externally. These particles may diffuse into tissue or remain in the vicinity of the cells.

Particles may also be used to enhance localization of transplanted (injected or implanted) cells e.g., reactive groups attached to cells may be used to immobilize cells within or on certain tissues or materials. In addition, particles with a higher degree of elasticity (e.g., soft particles) can be used to enhance the potential of the particles to localize by transporting through biological barriers (e.g., vascular endothelium).

Functionalized particles may be conjugated to cells in vitro or in vivo (conjugation in vivo may involve first targeting the particle to a particular cell in the body—particles could be delivered locally or systemically). In some embodiments, the particle contains a stealth ligand such as poly(ethylene glycol), hyaluronic acid, dextran, chitosan, or poly(ethylene oxide).

Properties of the particle that may be modified include, but are not limited to, shape, surface charge, porosity, chemical composition, relative hydrophobicity/hydropholicity, mechanical properties and surface texture. A particle may be modified through attaching biological (e.g., antibodies, peptides, nucleotides) or synthetic (e.g., small molecules, aptamers) molecules. Similar techniques can also be used to control the timing or location of activity. In addition, particles may further comprise one or more agents. The agents may be located (e.g., incorporated) within the particle (e.g., within pores or channels of the particle) and/or on the external surface of the particle. In some instances, the particles are pre-loaded with one or more agents. When the particle contains a ligand, it is preferred that the ligand does not interact with the cell directly, but rather the ligand interaction occurs with the particle only.

### Agents

A variety of different pharmaceutical/therapeutic agents can be used in conjunction with the methods described herein and include, but are not limited to, small molecules, proteins, antibodies, peptides and nucleic acids. In general, bioactive agents which can be administered via the invention include, without limitation: anti-infectives such as antibiotics and antiviral agents; chemotherapeutic agents (i.e. anticancer agents); anti-rejection agents; analgesics and analgesic combinations; anti-inflammatory agents; hormones (e.g., steroids); growth factors (e.g., bone morphogenic proteins (i.e. BMP's 1-7), epidermal growth factor (EGF), fibroblast growth factor (i.e. FGF 1-9), platelet derived growth factor (PDGF), insulin like growth factor (IGF-I and IGF-II), transforming growth factors (i.e. TGF-β-III), vascular endothelial growth factor (VEGF)); anti-angiogenic proteins such as endostatin, and other naturally derived or genetically engineered proteins, polysaccharides, glycoproteins, or lipoproteins. Additionally, the particles described herein can be used to deliver any type of molecular compound, such as for example, pharmacological agents, vitamins, sedatives, steroids, hypnotics, antibiotics, chemotherapeutic agents, prostaglandins, and radiopharmaceuticals. The cell compositions described herein are suitable for delivery of the above materials and others including, but not limited to, proteins, peptides, nucleotides, carbohydrates, simple sugars, cells, genes, anti-thrombotics, anti-metabolics, growth factor inhibitors, growth promoters, anticoagulants, antimitotics, fibrinolytics, anti-inflammatory steroids, drugs, and monoclonal antibodies.

Examples of other biologically active agents suitable for use in the methods described herein include, but are not limited to: cell attachment mediators, such as collagen, elastin, fibronectin, vitronectin, laminin, proteoglycans, or peptides containing known integrin binding domains e.g. “RGD” integrin binding sequence, or variations thereof, that are known to affect cellular attachment (Schaffner P & Dard 2003 Cell Mol Life Sci. January; 60(1):119-32; Hersel U. et al. 2003 Biomaterials November; 24(24):4385-415); biologically active ligands; and substances that enhance or exclude particular varieties of cellular or tissue ingrowth.

In some embodiments the particle comprises a drug for treatment of osteoporosis such as bisphosphonate based drugs, estrogen receptor modulators, or hormone based therapies. In certain embodiments the particle comprises a drug that inhibits osteoclast resorption such as raloxifene, AAR494, or E-64. In some embodiments the method further comprises a method for treating osteopenia. In some embodiments the method further comprises a method for treating bone cancer. In some embodiments the particle comprises a bone targeting factor such as a granulocyte colony-stimulating factor, or a bone marrow specific membrane surface receptor. In some embodiments the bone targeting factor is a factor such as pentosidine that targets osteoporotic bone. In some embodiments the method further comprises a method for targeting the bone marrow space.

In some embodiments the agent comprises an agent to promote bone growth. In certain embodiments the agent comprises a growth factor or a cytokine such as leptin, sortilin, transglutaminase, prostaglandin E, 1,25-dihydroxyvitamin D3, ascorbic acid, β-glycerol phosphate, TAK-778, statins, interleukins such as IL-3 and IL-6, growth hormone, steel factor (SF), activin A (ACT), retinoic acid (RA), epidermal growth factor (EGF), bone morphogenetic proteins (BMP), platelet derived growth factor (PDGF), hepatocyte growth factor, insulin-like growth factors (IGF) I and II, hematopoietic growth factors, peptide growth factors, erythropoietin, interleukins, tumor necrosis factors, interferons, colony stimulating factors, heparin binding growth factor (HBGF), alpha or beta transforming growth factor (α or β-TGF) such as TGF-β1, fibroblast growth factors, vascular endothelium growth factor (VEGF), nerve growth factor (NGF) and muscle morphogenic factor (MMP).

In some embodiments the particle comprises an agent that promotes the production or assembly of collagen such as pro-collagen or ascorbic acid. In certain embodiments the particle comprises a resorption factor for promoting particle resorption into bone such as receptor activator of NFκB ligand (Rank-L), a cortitosteroid such as dexamethasone, a parathyroid hormone, macrophage colony stimulating factor (M-CSF), or transforming growth factor-β1 (TGF-β1). In some embodiments the particle comprises an agent that chelates minerals from blood such as an EDTA-based agent, poly(bisphosphonate), poly(phosphate), biological or non biological entities that nucleate calcium and/or phosphate, aspartic acid, osteopontin, or bone sialoprotein.

While the cell compositions themselves can be thought of, in some aspects, as a carrier for delivery of a bioactive agent, the bioactive agents/therapeutics/pharmaceuticals of the cell compositions described herein can also impact, not only the tissue to be treated, but also the microenvironment of the cell composition, such that the cell composition itself becomes the bioactive agent. For example, the bioactive agent can be supplied in a particle for the purpose of enhancing the growth characteristics of the cell composition. This type of delivery is especially useful for targeted tissue regeneration, wherein the cell composition is used to re-populate the damaged tissue.

### Functionalization Methods

Several functionalization methods can be used with the methods described herein. Some exemplary embodiments include (1) reactions involving various functionalities on sugar residues on a cell surface, (2) reactions involving functional groups of a peptide residue on a cell surface, (3) using antigen-antibody interactions, (4) ionic interactions between a cell surface and a particle and (5) a hydrophobic interaction between a cell surface and a particle.

Particle based modification of a cell surface can be achieved by attaching particles with different functionalities to the cell membrane. The reactive sites and surface moieties present on the cell surface can be chemically modified by using different chemistries.

The physical characteristics of the cell surfaces can be manipulated similar to chemical modifications used to alter the interactions between cells and ECM. Cell surfaces have a complex structure, which exhibits different physical characteristics (e.g., charge). The different functionalities on the cell surface that can be utilized for attachment of particles or polymer chains include, but are not limited to: polar (NH2/NH3+) end groups and other functionalities of phospholipids, hydroxyls (OH) and other functionalities of carbohydrate groups, carboxylic acid groups (COOH), thiol (SH) and various protein and glycoprotein, antigens, among others. Particles (either solid or hollow) can be prepared with different ionic characters to adhere to cell membranes. Other types of physical adhesion are possible by regulating antigen-antibody interactions on a cell surface. The antibody can be functionalized for the purpose of specific applications.

Examples of cationic binding agents include, but are not limited to, chitosan, and polyamines, among others. Examples of anionic binding agents include, but are not limited to, polysulfonates, polyphosphates, DNA, heparin, and PMAM dendrimers, among others. Non-ionic binding includes for example, binding to carboxyl, amine, or sulphide functionalities.

One approach described herein involves chemical modification through the use of polymeric chains and particles. Organic polymeric and/or oligomeric particles can be used in this technology but it can also be extended to various inorganic particles as well. Chemical interactions between the particle (hollow or solid) surface and cell membrane can be achieved by various functionalities (e.g., biotinylation, N-hydroxysuccinimide (NHS), epoxy, peptides, imidoester, maleimide, azide, haloacetyl, pyridyl disulphide, carbodiimide, hydrazide) and chemical routes of cell surface modification described in the literature can be used and are known to those of skill in the art. Physical interactions can be performed, for example, by using charged polymers; self assembled charged particles, antibody-antigen, or hydrophobic interactions.

In this approach both solid and hollow particles can be used, which can contain important functional agents (e.g., growth factors, drug, enzymes, fluorescent moiety) to regulate/monitor the biomedical process (e.g., cell migration, adhesion, proliferation, differentiation, survival, matrix production). Molecules, distinct from the macromolecules of which the particles are composed, may be attached to the outer surface of the particles by methods known to those skilled in the art to “coat” or “decorate” the particles. The molecules may be attached directly or indirectly to the outer surface of the particle for instance through the use of a linker (discussed below). These molecules are attached for purposes such as to facilitate binding, enhance receptor mediation, and provide escape from endocytosis or destruction. For example, biomolecules such as phospholipids may be attached to the surface of the particle to prevent endocytosis by endosomes; receptors, antibodies or hormones may be attached to the surface to promote or facilitate binding of the particle to the desired organ, tissue or cells of the body; and polysaccharides, such as glucans, or other polymers, such as polyvinyl pyrrolidone and PEG, may be attached to the outer surface of the particle to enhance or to avoid uptake by macrophages.

Functionalization of cells in solution may be used to promote homogenous functionalization and/or to stimulate cell aggregation whereas functionalization of cells on a surface may be useful to preferentially functionalize one surface of the cell.

Addition of particles to cells may be achieved under static or dynamic conditions and may include the use of bioreactors and/or BioMEMS devices including microfluidic channels. Cells with particles could be used as an inhalant for pulmonary delivery of cells with particles.

### Material Selection

The choice of material for attaching a particle to form a cell composition as described herein, depends on the type of modification deemed necessary for the desired application by one of skill in the art. Synthetic, natural, as well as semi-synthetic polymers, can be used for the synthesizing the polymeric particles. Different synthetic polymers include, for example, hydrogel polymers (PEG, PVA etc.), or acrylates. These polymers can be linear or crosslinked according to the needs of one skilled in the art. Natural polymers that can be used include, but are not limited to, hyaluronic acid, gelatin, chitin, etc. For physical interactions several polymers including, for example poly ethylene imines (PEI), poly (lysine), chitosan, or cellulose can be used for charge based adhesion to the cell surface. The list of polymers that can be used includes, but is not limited to, biodegradable polymers such as poly(lactide) (PLA), poly(glycolide) (PGA), poly(lactide-co-glycolide) (PLGA), poly(caprolactone) (PCL), polycarbonates, polyamides, polyanhydrides, polyphosphazene, polyamino acids, polyortho esters, polyacetals, polycyanoacrylates and biodegradable polyurethanes; non-biodegradable polymers such as polyacrylates, ethylene-vinyl acetate polymers and other acyl-substituted cellulose acetates and derivatives thereof; polyurethanes, polystyrenes, polyvinyl chloride, polyvinyl fluoride, poly(vinyl imidazole), chlorosulphonated polyolefins, and polyethylene oxide. Examples of biodegradable natural polymers include proteins such as albumin, collagen, synthetic polyamino acids and prolamines; polysaccharides such as alginate, heparin; and other naturally occurring biodegradable polymers of sugar units. Alternately, combinations of the aforementioned polymers can be used.

Examples of inorganic particles that can be used include, but are not limited to the following: titanium dioxide, calcium carbonate, calcium phosphate, calcium silicate, silver and gold nanoparticles, and magnetic particles, among others. Different types of particles with a wide range of geometries that are useful for the methods described herein may be used. A non limiting list of particle shapes includes, for example core-shell material, hollow particles, cage like particles, among others.

### Linkers

Linkers may include functional groups such as a hydroxyl group, a primary or secondary amino group, a phosphate group or substituted derivatives thereof or a carboxylic acid group. Polar lipids such as acyl carnitine, acylated carnitine, sphingosine, ceramide, phosphatidyl choline, phosphatidyl glycerol, phosphatidyl ethanolamine, phosphatidyl inositol, phosphatidyl serine, cardiolipin and phosphatidic acid may also function as linkers. The polar lipid molecules may optionally be covalently linked to an organic spacer molecule which may or may not have functional groups. Other linkers include heterobifunctional cross-linkers. For instance, succinimidyl-4-(N-maleimidomethyl)cyclohexane-1-(carboxy-6-aminocaproate)-, also known as LC-SMCC, is a heterobifunctional cross-linker that reacts with sulfhydryl group and amine-groups. Non-limiting examples of linkers include the polymeric anionic, cationic and nonionic agents described above.

Fabrication of particles can be performed by a variety of techniques. Solvent evaporation of emulsion, or spray drying can be applied to fabricate the particles. Depending on whether a solid or hollow particle is desired, multiple emulsion techniques can be used. Chemical modifications can be performed by using a variety of chemical reactions, which depends on the side group or functionality required.

In some embodiments, the linker comprises a stealth ligand such as poly(ethylene glycol), hyaluronic acid, dextran, chitosin, or poly(ethylene oxide).

Cross-Linking Particles and/or Ligands to a Cell

### Methods Used to Study the Cell Membrane

The structural composition, distribution, cellular association and dynamics of cell membrane molecules can be studied by using different chemistries. Different chemical crosslinkers are able to crosslink cellular and organellar membranes both at the outer surface of a membrane and within the membrane-bounded space. Crosslinkers are often used to identify surface receptors or their ligands. Membrane-impermeable crosslinkers ensure cell-surface specific crosslinking. Water-insoluble crosslinkers, when used at controlled amounts of reagent and reaction time, can reduce membrane penetration and reaction with inner membrane proteins. The sulfonyl groups attached to the succinimidyl rings of NHS-esters result in a crosslinker that is water-soluble, membrane impermeable and nonreactive with inner-membrane proteins. Therefore, reaction time and quantity of crosslinker are less critical when using sulfo-NHS-esters.

Cell membrane structural studies require reagents of varying hydrophobicity to determine the location and the environment within a cell's lipid bilayer. Fluorescent tags are used to locate proteins, lipids or other molecules inside and outside the membrane. Various crosslinkers, with differing spacer arm lengths, can be used to crosslink proteins to associated molecules within the membrane to determine the distance between molecules. Successful crosslinking with shorter crosslinkers is a strong indication that two molecules are interacting in some manner. Failure to obtain crosslinking with a panel of shorter crosslinkers, while obtaining conjugation with the use of longer reagents, generally indicates that the molecules are located in the same part of the membrane, but are not interacting. Homo-bifunctional NHS-esters, imidotesor hetero-bifunctional NHS-ester/photoactivatable phenyl azides are commonly used for these procedures. Although imidoester crosslinkers (imidates) are water-soluble, they are still able to penetrate membranes. Sulfhydryl-reactive crosslinkers maybe useful for targeting molecules with cysteines to other molecules within the membrane. EDC, water-insoluble dicyclohexyl carbodiimide, DCC, and other water-soluble/-insoluble coupling reagent pairs are used to study membranes and cellular structure, protein subunit structure and arrangement, enzyme: substrate interactions, and cell-surface and membrane receptors. The hydrophilic character of EDC can result in much different crosslinking patterns in membrane and subunit studies than with hydrophobic carbodiimides such as DCC. In one embodiment one can crosslink with a water-soluble and water-insoluble carbodiimide to obtain a complete picture of the spatial arrangements or protein: protein interactions involved. These crosslinking methods are useful in the compositions described herein, to test the location and interaction of functionalized particles and/or ligands on the surface of a cell.

### Methods for Performing Crosslinking Reactions on the Cell Surface

Crosslinkers can be used to study the structure and composition of proteins in samples. Some proteins are difficult to study because they exist in different conformations with varying pH or salt conditions. One way to avoid conformational changes is to crosslink subunits. Amine-, carboxyl- or sulfhydryl-reactive reagents are used for identification of particular amino acids or for determination of the number, location and size of subunits. Short- to medium-spacer arm crosslinkers are selected when intramolecular crosslinking is desired. If the spacer arm is too long, intermolecular crosslinking can occur.

Carbodiimides that result in no spacer arm, along with short-length conjugating reagents, such as amine-reactive or the photoactivatable amine-reactive crosslinker, can crosslink between subunits without crosslinking to extraneous molecules if used in optimal concentrations and conditions.

Slightly longer crosslinkers can also crosslink between subunits, but they may result in intermolecular coupling. Adjusting the reagent amount and protein concentration can control intermolecular crosslinking. Dilute protein solutions and high concentrations of crosslinker favor intramolecular crosslinking when homobifunctional crosslinkers are used.

For determination or confirmation of the three-dimensional structure, cleavable crosslinkers with increasing spacer arm lengths can be used to determine the distance between subunits. Experiments using crosslinkers with different reactive groups may indicate the locations of specific amino acids. Once conjugated, the proteins are subjected to two-dimensional electrophoresis. In the first dimension, the proteins are separated using non-reducing conditions and the molecular weights are recorded. Some subunits may not be crosslinked and will separate according to their individual molecular weights. Conjugated subunits will separate according to the combined molecular weight. The second dimension of the gel is then performed using conditions to cleave the crosslinked subunits. The individual molecular weights of the crosslinked subunits can be determined. Crosslinked subunits that were not reduced will produce a diagonal pattern, but the cleaved subunits will be off the diagonal. The molecular weights of the individual subunits should be compared with predetermined molecular weights of the protein subunits using reducing SDS-polyacrylamide gel electrophoresis. This crosslinking technology allows engineering the cell surface with various functionalities for the required biospecific application.

Homobifunctional sulfo-NHS esters, heterobifunctional sulfo-NHS-esters and photoreactive phenylazides are used in more preferred embodiments for crosslinking proteins on the cell surface. Determination of whether a particular protein is located on the surface or the integral part of the membrane can be achieved by performing a conjugation reaction of a cell membrane preparation to a known protein or radioactive label using a water-soluble or water-insoluble crosslinker. Upon conjugation the cells may be washed, solubilized and characterized by SDS-polyacrylamidegel electrophoresis (PAGE) to determine whether the protein of interest was conjugated. Integral membrane proteins will form a conjugate in the presence of a water-insoluble crosslinker, but not in the presence of water-soluble crosslinkers. Surface membrane proteins can conjugate in the presence of water-soluble and water-insoluble crosslinkers. Homobifunctional photoactivatable phenyl azide is one of the more versatile crosslinkers for the study of protein interactions and associations. It is cleavable and can be radiolabeled with ¹²⁵I. After cleavage, both of the dissociated molecules will still be iodinated. Because both reactive groups on this crosslinker are nonspecific, the crosslinking is not dependent on amino acid composition for successful conjugation.

### Cell Administration

A variety of means for administering cells to subjects are known to those of skill in the art. Such methods can include systemic injection, for example i.v. injection or implantation of cells into a target site in a subject. Cells may be inserted into a delivery device which facilitates introduction by injection or implantation into the subjects. Such delivery devices may include tubes, e.g., catheters, for injecting cells and fluids into the body of a recipient subject. In one preferred embodiment, the tubes additionally have a needle, e.g., a syringe, through which the cells of the invention can be introduced into the subject at a desired location. The cells may be prepared for delivery in a variety of different forms. For example, the cells may be suspended in a solution or gel or embedded in a support matrix when contained in such a delivery device. Cells may be mixed with a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention may be prepared by incorporating cells as described herein in a pharmaceutically acceptable carrier or diluent and, as required, other ingredients enumerated above, followed by filtered sterilization.

It is preferred that the mode of cell administration is relatively non-invasive, for example by intravenous injection, pulmonary delivery through inhalation, oral delivery, buccal, rectal, vaginal, topical, or intranasal administration. However, the route of cell administration will depend on the tissue to be treated and may include implantation. Methods for cell delivery are known to those of skill in the art and can be extrapolated by one skilled in the art of medicine for use with the methods and compositions described herein.

Direct injection techniques for cell administration can also be used to stimulate transmigration through the entire vasculature, or to the vasculature of a particular organ, such as for example liver, or kidney or any other organ. This includes non-specific targeting of the vasculature. One can target any organ by selecting a specific injection site, such as e.g., a liver portal vein. Alternatively, the injection can be performed systemically into any vein in the body. This method is useful for enhancing stem cell numbers in aging patients. In addition, the cells can function to populate vacant stem cell niches or create new stem cells to replenish the organ, thus improving organ function. For example, cells may take up pericyte locations within the vasculature.

Delivery of cells may also be used to target sites of active angiogenesis. For example, delivery of endothelial progenitor cells or mesenchymal stem or progenitor cells may enhance the angiogenic response at a wound site. Targeting of angiogenesis may also be useful for using cells as a vehicle to target drugs to tumors.

If so desired, a mammal or subject can be pre-treated with an agent, for example an agent is administered to enhance cell targeting to a tissue (e.g., a homing factor) and can be placed at that site to encourage cells to target the desired tissue. For example, direct injection of homing factors into a tissue can be performed prior to systemic delivery of ligand-targeted cells.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. In addition, the term ‘cell’ can be construed as a cell population, which can be either heterogeneous or homogeneous in nature, and can also refer to an aggregate of cells.

The described technology can be applied to improve the engraftment efficiency of embryos, for example during in vitro fertilization embryos can be modified with adhesion ligands that could enhance attachment to specific tissues (e.g., uterus).

It is understood that the foregoing detailed description and the following examples are illustrative only and are not to be taken as limitations upon the scope of the invention. Various changes and modifications to the disclosed embodiments, which will be apparent to those skilled in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### EXAMPLES

### Example 1

Described herein is a non-limiting example of how a cell can be modified to improve cell targeting. Specifically, a targeting agent was attached to the cell surface, which is able to induce cell rolling as demonstrated by in vitro experiments.

Modification of Human Mesenchymal Stem Cells (hMSCs) by Sialyl Lewis X (SLeX)

The modification of hMSCs was performed to chemically attach the SLeX to surface of the cell membrane. Biotin-Streptavidin conjugation was utilized to chemically incorporate the SLeX moiety of the cell surface. The free amine groups present on the surface of the cells were allowed to react with N-hydroxy-succinimide group of Biotinyl-N-hydroxy-succinimide to biotinylate the cell surface. This step was subsequently followed by reacting the biotin moiety of the cell surface with a streptavidin molecule. The strong interaction between biotin and streptavidin allows the streptavidin molecule to be immobilized on the cell surface. The streptavidin is then reacted with biotinylated SLeX (Sialyl-Lex-PAA-Biotin) to introduce SLeX on the cell surface. Since the absence of targeting agents such as SLeX on the surface of hMSCs is responsible for their non-rolling characteristics, the immobilization of SLeX on the cell can induce rolling characteristics to hMSCs. To improve the biotinylation step, sulfonated biotinyl-N-hydroxy-succinimide (Sulfo-NHS-Biotin, BNHS) was used. BNHS is water soluble and impermeable through the cell membrane, and thus allows the N-hydroxy-succinimide group of BNHS to interact preferentially with the cell membrane.

### General Methods

Typically, about 5000 cells were added to each well of a 96 well plate in 200 μL hMSC cell media (15% Fetal Bovine Serum, 1% L-Glutamine, 1% Penn-Strep in α-MEM) for a period of 24-48 hours so that the cells were ˜80-90% confluent.

To treat the cells, media was aspirated from the wells and the cells were gently washed with 200 μL of phosphate buffer saline (PBS, pH 7.4 without Ca/Mg) at room temperature in each well. After that 100 μL of 1 mM BNHS solution in PBS, pH 7.4 without Ca/Mg was added to each well for 20-25 minutes at room temperature. After a designated time period, the cells were washed twice with 200 μL of phosphate buffer saline (PBS, pH 7.4 without Ca/Mg) at room temperature. Then 50 μL of streptavidin solution (50 μg/mL in PBS, pH 7.4 without Ca/Mg) was added to each well and incubated for 20 minutes at room temperature. After a designated time period, the cells were washed twice with 200 μL of phosphate buffer saline (PBS, pH 7.4 without Ca/Mg) at room temperature, followed by the addition of 50 μL of 4 μg/mL SLex (in PBS, pH 7.4 without Ca/Mg) at room temperature.

To assess the cell modification by BNHS and streptavidin, fluorescent rhodamine conjugated streptavidin (SR) was added to biotinylated cells and the fluorescence intensity of SR conjugation was measured. The controls used for this experiment were cells treated with only SR, cells treated with biotin and SR and untreated cells. The fluorescence intensity was measured again after 7 days and the cells were fixed with 4% formaldehyde solution and stained with 100 μL of DAPI solution (1 μg/mL in PBS) after being treated with 100 μL of 0.1% TRITON X solution in PBS. Fluorescence intensity of cell treated with a biotin analog was measured on day 0 and day 7, following functionalization. The fluorescence intensity of BNHS-SR conjugated cells was higher than that for B-SR or SR treatment alone. All the results reported are for the experiments done in triplicate.

### Viability of the Cells

The viability of the cells was performed using Trypan blue exclusion. Briefly, cells were plated into 12 well plates, left to adhere over night and the cells were treated with BNHS solution as described above. After rinsing, the cells were incubated for 48 hours at 37° C. and 5% CO₂. The media was then aspirated and the cells were detached from the well by using 200 μL cell dissociation solution. 300 μL of media was added and the total 500 μL of the cell suspension was collected. From this, 10 μL of cell suspension was diluted to 1:1 by using 4% Trypan blue solution and cells were counted in a hemocytometer to determine the number of viable (unstained) and nonviable (blue-stained) cells. There were no differences in the mean cell viability calculated from triplicate experiments among the groups. The controls for this experiment included cells with no treatment (but PBS was added during the experiment and kept in room temperature) and the cells treated with biotin and SR without NHS.

The viability of the cells modified with BNHS shows that treatment with BHS does not change the cell viability even after 48 hours of modification. The untreated cells were 85% viable after 48 hours, whereas cells treated with Biotin were 76% viable and cells treated with BNHS were 75% viable. This indicates that modification of the cells by BNHS was not toxic to the cells, since cells were viable after 48 hours of the modification.

### Stability and Accessibility of Biotin on the Biotinylated Cells

To analyze the stability of the biotin on the cell surface, cells were biotinylated as described above. Biotin treated cells without NHS were used as controls. 200 μL of SR solution (50 μg/mL in PBS, pH 7.4 without Ca/Mg) was added for 20 minutes at room temperature on either Biotin or BNHS treated cells on day 0, 2, 4 and 7 to assess the stability of biotin on the cell surface. After addition of SR, the cells were fixed with 4% formaldehyde solution and stained with 100 μL of DAPI solution (1 μg/mL in PBS) after being treated with 100 μL of 0.1% TRITON X solution in PBS. The fluorescent microscopic images of both the BNHS and Biotin treated cells were analyzed by measuring the fluorescence intensity. The stability of the biotin functionality was measured by analyzing the fluorescence intensity of the added rhodamine-streptavidin.

The stability of biotin on the cell surface was analyzed by adding rhodamine-streptavidin at each time point and immediately measuring the fluorescence intensity of the images. The stability of the BNHS functionalization over 7 days was tested compared to biotin without NHS (control). NHS treatment was demonstrated to provide stable biotin groups on the cell membrane over 7 days.

The fluorescence intensity of cells functionalized with BNHS is higher on day 0 (the day of the functionalization) compared to B (biotin) and SR, which indicates that the cell has been functionalized by the NHS functionality. After 7 days the intensity was re-examined and found to be significantly elevated for BNHS+SR treatment compared to B+SR and SR controls indicating that the conjugation of rhodamine to the biotinylated cells was still present. The representative 7 day light microscope images and corresponding fluorescent images of the same field indicates that cells were effectively biotinylated via the NHS chemistry and the conjugated streptavidin added on day 1 was stable for 7 days of culture.

### Cell Adhesion

Cell adhesion was analyzed by measuring the number of adherent cells on tissue culture wells after biotinylation of the cells. 80% confluent cells in a T25 flask were biotinylated as described above. After streptavidin conjugation, the cells were washed and detached from the flask using cell dissociation solution. The cells were counted in a hemocytometer and then 5000 cells were plated into each well of a 96 well plate for 10, 30 and 90 minutes. After which, the non-adhered cells were removed by rinsing twice with PBS followed by fixing the adhered cells and staining them with toluidine blue solution. The adhered cells were counted in 6 fields of 3 wells at 10× magnification to determine the number of adherent cells. The percentage of cell adhered to the surface was calculated based on the initial seeding density of the cells. A control for this experiment included cells with no treatment.

There was no difference in percent adherent cells for the BNHS treated cells and the controls, indicating that modification of the cells by BNHS does not alter the adhesion characteristics of the cells. Compared to untreated cells after 10, 30 and 90 minutes of adhesion on tissue culture plastic respectively, the modified cells do not exhibit any significantly different adherent characteristics. This clearly indicates that the cell surface receptors responsible for the adherent characteristic of cells (e.g., integrins) are not affected by the NHS biotin cell surface modification.

### Proliferation Assay of Modified Cells

Cell proliferation analyzed by measuring the number of cells on a T25 flask, using a cell proliferation assay. 80% confluent cells in a T25 flask were treated with 1 mL of 1 mM BNHS followed by treatment with 1 mL of 50 μg/mL of streptavidin solution for a 20 minute time period. The cells were then washed and detached from the flask using a cell dissociation solution. The cells were counted in a hemocytometer and 50,000 cells were plated into three T25 flasks. The number of cells was assessed on day 1, 2, 4 and 6 in 10 fields of 3 flasks in 10× magnification after the seeding. A growth curve indicating proliferation characteristics of the cells modified by BNHS indicate proliferation eventually leading to confluent monolayering in both groups.

Functionalization of Cells with SLeX and Resulting Rolling Characterization

Modification of cells was performed by treating 80% confluent cells in a T25 flask. Cells were treated with 1 mL of 1 mM BNHS followed by treatment with 1 mL of 50 μg/mL of streptavidin solution for the 20 minute time period. The cells were washed twice with 1 mL of PBS and then 1 mL of 4 μg/mL SLeX (in PBS, pH 7.4 without Ca/Mg) was added at room temperature for 20 minutes. Cells were detached from the flask using a cell dissociation solution and re-dispersed in culture media after centrifugation. The cell concentration for the flow chamber experiment was typically 1×10⁵ cell/ml. The rolling characteristics of the cells was assessed by a rectangular parallel plate flow chamber experiment with 127 μm gasket thickness and a length of 6 cm. P-selectin immobilization on a glass surface was performed by incubating 700 μL of P-selectin solution (5 μg/mL) on a glass slide for 18 hours and flow experiments were performed by placing the chamber on the glass slide. The flow rate of 20 μL/min corresponding to wall shear stress 0.094 dyne/cm² was used. To monitor rolling of the cells, phase contrast microscopy was used and the images were recorded in a 10× field and captured manually approximately every 10 seconds. The velocity of the cells was calculated by measuring the distance of the moving cells over a 10 second time period. The control was untreated cells and cells with SLeX physically adsorbed onto the cell surface. Cells modified with SLx (SLeX) or BNHS-SLx exhibit reduced velocities and increased rolling characteristics. To assess the effect of shear stress and the concentration of BNHS added to the cells, the rolling velocity and the flux of the cells were measured in flow rates (20, 40 and 100 μL/min corresponding to 0.366, 0.73 and 1.89 dyne/cm² respectively).

It has been well described that cultured hMSCs do not express ligands on their surface to enable cell rolling and these results were confirmed for untreated hMSCs. The cells modified with BNHS followed by SLeX show considerably lower velocity than untreated cells and cells with physically adsorbed SLeX on the cell surface at a wall shear stress of 0.366 dynes/cm². Compared to untreated cells, the significantly lower velocity of SLeX immobilization indicates that the interaction between SLeX and P-selectin reduces the velocity to around 2 μm/s at a wall shear stress of 0.366 dynes/cm². This indicates that modification of the cell by biotin-streptavidin followed by SLeX-biotin induces rolling characteristics of hMSCs that are not inherent to this cell type.

The effect of flow rates and thus different shear stresses on the rolling characteristics of the modified cells was measured. As shear stress is increased from 0.366 dynes/cm² to 1.88 dynes/cm² the velocity of the modified cells increases whereas the flux (number of interacting cells with the substrate) remains for the most part unchanged (at constant 1 mM BNHS concentration). Unmodified cells displayed a constant flux of 19.5 cells/mm²·sec. This indicates that cells modified with SLeX specifically interact with P-selectin on the surface via recognized cell rolling based adhesion phenomena.

The effect of BNHS concentration on the rolling characteristics of the modified cells at a constant shear stress of 0.366 dyne/cm² (essentially this is the density control of modified ligand on the cell surface) was also assessed. With decreasing concentrations of BNHS from 1 mM to 0.01 mM, the velocity increases whereas the flux of the modified cells is almost unchanged (and higher than the flux of the unmodified cells which displayed a flux of 19.5 cells/mm²·sec). This indicates the ability to control the rolling response through altering the concentration of ligand on the cell surface. Even when low concentrations of BNHS are used, the cells can interact with P-selectin as observed by the flux data.

The modification of the cells by BNHS and subsequent streptavidin and SLex conjugation was performed on adhered cells on a tissue culture plate. This modification of the cell surface, only modified a portion of the cell that is not bound to the surface and is thus exposed to ligands for conjugation; the cell surface that is adhered to the tissue culture plate is not exposed to modification. This leads to half of the cell surface being modified by SLex. Thus the adherence of the SLex modified cells on P-selectin surfaces is more prominent than the rolling velocity. This explains why the cells have unchanged flux values even at different conditions of shear stress and concentration of BNHS.

### Differentiation Potential of the Modified Cells

In vitro osteogenic and adipogenic differentiation potential of cells modified by biotin/streptavidin conjugation was assessed, as described below.

### Osteogenic Differentiation

hMSCs were seeded in 4 wells of a 24 well plate and were cultured in a cell expansion media until reaching 90% confluence. The modification of the cells was performed by a two step method. Typically the cells were biotinylated with 1 mM BNHS followed by conjugation with streptavidin (50 μg/mL) in PBS at room temperature. Hereafter, the osteogenic differentiation was induced by culturing the cells for 23 days in osteogenic induction media (from Lonza—hMSCs Osteogenic Single Quote kit) containing dexamethasone, β-glycerophosphate, L-ascorbic acid-2-phosphate, and α-MEM. As a positive control, an equal number of wells with untreated (un-modified) cells were maintained in osteogenic induction medium. The media in both groups was changed every 3 days. Osteogenesis was evaluated by alkaline phosphatase assay and von Kossa staining.

The alkaline phosphatase assay was performed by aspirating the medium and washing the cells with distilled water. The cells were fixed with 3.7% formaldehyde solution for 15 min at room temperature and then washed twice with distilled water. To it 0.06% Red Violet LB salt solution in Tris HCl and distilled water containing (DMF and Naphthol AS MX-PO4) was added. The plates were incubated for 45 minutes and then the wells were rinsed 3 times with distilled water.

The osteogenic differentiation potential of cells modified by biotin and streptavidin was measured by alkaline phosphatase staining. The alkaline phosphatase staining for the modified cells shows positive staining and comparable results with unmodified cells. This indicates that the biotin-streptavidin modification of the cells does not interfere with the osteogenic potential of the cells and modified cells can differentiate into their osteogenic lineages.

### Adipogenic Differentiation

hMSCs were seeded in 4 wells of 24 well plate and were cultured in a cell expansion media until reaching 100% confluence. The modification of the cells was performed by a two step method. Typically the cells were biotinylated with 1 mM BNHS followed by conjugation with streptavidin (50 μg/mL) in PBS at room temperature. Hereafter, the adipogenic differentiation was induced by culturing the cells for 23 days in adipogenic induction media (from Lonza—hMSCs Adipogenic Single Quote kit containing h-Insulin (recombinant), L-Glutamine, Dexamethasone, Indomethacin, IBMX (3-isobuty-1-methyl-xanthine), Pen/Strep) and adipogenic maintenance media (from Lonza—hMSCs Adipoogenic Single Quote kit containing h-Insulin (recombinant), L-Glutamine, Pen/Strep). As a positive control, an equal number of wells with untreated (un-modified) cells were maintained in adipogenic induction medium. The media in both groups was changed every 3 days according to Lonza protocol in a periodical exposure of induction and maintenance media. Adipogenesis was evaluated by Oil Red O staining.

The Oil Red O staining protocol used is as follows: aspirate all of the media off of the cells, wash once with PBS, replace the PBS with 3.7% formaldehyde for 30 min at room temperature to fix the cells, replace the formaldehyde with distilled water for a few minutes, replace isopropanol with Oil Red O working solution (made by diluting 30 ml of 0.5% isopropanol solution of Oil Red with 20 ml distilled water), after 5 minutes the Oil red O solution was washed twice with distilled water. One ml of hematoxylin (Sigma-Aldrich) was added to the well for 1 minute before being aspirated and the wells were washed with distilled water. The wells were viewed using an inverted phase contrast microscope. Lipids appeared red and nuclei appeared blue.

The adipogenic differentiation potential of cells modified by biotin and streptavidin was assessed by Oil Red O and hemotoxylin staining and results indicate that modified cells show positive staining and comparable results with unmodified cells. This indicates that the biotin-streptavidin modification of the cells does not interfere with the adipogenic potential of the cells and modified cells can differentiate into their adipogenic lineages.

### Example 2

The methods described herein in Example 2 can be used for targeted delivery of any cell type including, for example stem cells and differentiated cells. Activated dendritic cells (DC) presenting specific antigens can be targeted to the lymph nodes to improved vaccination strategies⁴³. In addition, targeted delivery of T-cells or other immune cells can be performed by the methods described herein. Encapsulation of drugs or drug delivery devices (e.g., particles) into the cell surface is also useful to control the cell microenvironment and to deliver drugs directly to the cell (over long term). This is particularly useful for drugs that are quickly cleared, or inactivated, by interaction with plasma or other biological entities. Sustained drug delivery can be achieved through covalent immobilization to the cell surface or through incorporation by non-covalent methods described herein.

This method describes one embodiment of the methods disclosed herein and involves the covalent functionalization of human mesenchymal stem cells and can be applied to any cell type.

In this embodiment cell functionalization is achieved without direct covalent attachment to the cell surface. For this, the following biotinylated lipids can be used: lipids with different headgroups with varying charge such as neutral, cationic and anionic; lipids with varying length of hydrocarbon chain (see Table 1); and lipids with various degree of unsaturation in hydrocarbon chains.

This platform approach is superior to existing functionalization methods as it provides multiple advantages including: i) simple preparation methods, ii) mild reaction conditions, iii) avoidance of expensive/complicated protein expression steps, iv) reduced time and manipulation of cells which can be used in a kit.

Two different approaches to achieve the same goal i.e., functionalization of cells with specific ligands can be used. In one approach, streptavidin is added to a vesicle having biotinylated lipids, and Sialyl Lewis X is further added to the streptavidin coated cells. The vesicle is then fused with the bilayer of a cell to be modified. In the second approach, biotinylated lipid vesicles are first fused with a cell to be modified, and then the streptavidin and subsequently the Sialyl Lewis X are added to the cell. The end-product of both approaches is essentially identical.

### Method 1

In method one; first unilamellar or/and multilamellar vesicles can be made using either only ‘biotinylated lipids’ or ‘biotinylated lipids with a supporting lipid with different ratios’ in phosphate buffered saline (PBS, pH 7.4) or any other aqueous solution, are added to a streptavidin solution (which can bind to biotin) and incubated at room temperature for 5-30 mins, the vesicle solution is centrifuged for 2 mins at 10,000 rpm followed by removal of the supernatant. The pellet (containing vesicles) is washed with PBS to remove unbound streptavidin. Biotinylated Sialy Lewis X (SiLeX) was added to the pellet and incubated for 5-30 mins, then the centrifugation and PBS washing steps were repeated to obtain vesicles, which are coated with biotin-streptavidin-biotin-SiLeX group at the lipid headgroup. Finally, vesicles were incubated with hMSCs and/or another cell types for 1-15 mins. Fusion of vesicles with cell membrane occurs and the surface of the cells can be coated with biotin-streptavidin-biotin-SiLeX groups, which are susceptible to exclusive interaction with selectins (in this case P-selectin).

In this approach, vesicles were prepared using biotinylated lipids by the following method a) strepatavidin solution was incubated with the mixture for 5-30 mins followed by centrifugation; removal of excess unbound streptavidin allowed preparation of vesicles having biotin-streptavidin functionalization on the surface, b) further biotinylated SiLeX was added to the vesicles and was incubated for 5-30 mins, centrifugation and removal of excess (un-fused) vesicles was performed, and the remaining vesicles contain biotin-streptavidin-biotin-SiLeX groups on the surface. c) Such vesicles were incubated with human mesenchymal stem cells (hMSCs) (or other cell types) for 1-15 mins, and the resulting fusion of vesicles with cell membrane caused functionalization of cell surface with biotin-streptavidin-biotin-SiLeX groups, which are susceptible for exclusive interaction with selectins (in this case P-selectin).

### Method 2

In method two; vesicles were prepared using biotinylated lipids, incubated with hMSCs (or any other cell type) suspensions for 1-15 mins in PBS, then the cells were centrifuged for 3 mins at 10,000 rpm, the supernatant was removed, and the resulting pellet contains biotin coated cells. Biotin coated cells were incubated for 5-30 mins with a streptavidin solution, followed by centrifugation and removal of unbound streptavidin, which allows coating of the cell surface with biotin-streptavidin groups. Incubate the cells for 5-30 mins with biotinylated SiLeX, repeat centrifugation and removal of excess reagent as in the previous step, thus producing cells which have biotin-streptavidin-biotin-SiLeX groups on the surface.

In this approach, vesicles were prepared using biotinylated lipids by the following protocol: a) incubated with either hMSCs or any other cell types for 1-15 mins, then cells were centrifuged for 3 mins at 10,000 rpm, the supernatant was then removed, and the resulting pellet contains biotin coated cells, b) to those cells a streptavidin solution was added and incubated for 5-30 mins, followed by centrifugation removal of unbound streptavidin. This method allowed coating of a cell surface with biotin-streptavidin groups, c) biotinylated SiLeX was added and incubated for 5-30 mins, and the cells were subjected to centrifugation and removal of excess reagent. This method lead to preparation of cells which have biotin-streptavidin-biotin-SiLeX groups on the surface. Similarly steps can be done on adhered cells on culture plate as well.

Depending on the desired vesicle properties, various phospholipids with different headgroups and different charges (e.g., positive, negative, neutral and zwetter ionic) can be used. For example, phosphatidylcholine, phosphatidylserine and phosphatidylethanolamine based lipids can be used to make vesicles. Varying hydrophobic chains are also contemplated for use in the methods described herein, for example symmetrical and asymmetrical acyl groups attached to the glycerol backbone. Table 1 lists asymmetrical acyl groups and symmetrical acyl groups which are contemplated herein.

This methodology is not limited only to biotinylated lipids, but rather cell functionalization can be performed with any ‘modified lipid’. Desired functional group/molecules/nanoparticles/beads can be attached to a lipid headgroup and then inserted into any type of cell surface. In addition, the desired drugs/molecules/growth factors/particles/beads can be encapsulated into vesicles, which are prepared by functionalized lipids, thus encapsulated material can be delivered into the cells while simultaneously coating the surface with functionalized lipids.

### Example 3

### Cell Surface Functionalization of Cells in Suspension

Modification of Human Mesenchymal Stem Cells (hMSCs) by Sialyl Lewis X (SLeX) in Suspension Mode

The covalent modification of hMSC by reacting N-hydroxy-succinimide group of Biotinyl-N-hydroxy-succinimide in suspension offers an opportunity to modify the entire surface of the cell whereas in adherent mode approximately half of the cell surface is exposed during the surface modification. Thus, the distribution of biotin and conjugated moieties is more uniform throughout the cell membrane when modifications are performed on cells in suspension. This can increase a cell's efficiency for targeting and homing. Specifically, the attachment of SLeX while cells were in suspension imparted a more robust rolling response compared to modifications performed on adherent cells (when reagents were used at the same concentrations).

Typically, hMSCs were cultured in a T75 flask up to 90% confluence in hMSC expansion media (15% Fetal Bovine Serum, 1% L-Glutamine, 1% Penn-Strep in a-MEM), after which the cells were trypsinized using 1× trypsin-EDTA solution and then washed with phosphate buffer saline (PBS without Ca/Mg, pH 7.4) to remove media and trypsin. The cell pellet was then dispersed in 1 mL of 1 mM BNHS solution for 15-20 minutes. After that the cells were centrifuged and spun down to remove the BNHS solution. The cell pellets were washed with PBS twice by resuspending the cells followed by centrifugation. After the centrifugation, the cell pellet was re-suspended in 1 mL of streptavidin solution (50 μg/mL in PBS, pH 7.4 without Ca/Mg) for 15-20 minutes. The unconjugated streptavidin was removed by centrifugation.

To assess the cell modifications by BNHS and streptavidin, fluorescent rhodamine conjugated streptavidin (SR) was added to biotinylated cells and the fluorescence intensity of conjugated SR was monitored. The controls used for this experiment were the cells treated with only SR, cells treated with biotin and SR, and cells with no treatment. The fluorescent intensity was measured again after 7 days and the cells were fixed with 4% formaldehyde solution and stained with 100 μL of DAPI solution (1 μg/mL in PBS) after being treated with 100 μL of 0.1% TRITON X solution in PBS. All the results reported are for the experiments performed in triplicate. On the day of modification (day 0) the BNHS+SR group had significantly higher fluorescence, which further increased on day 7 (compared to control SR and B+SR). This indicates that BNHS specifically and covalently binds to the cell surface leading to permanent modification of the cell surface.

### Stability and Accessibility of Biotin on the Biotinylated Cells:

To analyze the stability of biotin on the cell surface, cells were biotinylated as described above. Biotin treated cells without NHS were used as controls. The cells were allowed to adhere in 96 well plates for 24 hours. After the designated time period, 200 μL of SR solution (50 μg/mL in PBS, pH 7.4 without Ca/Mg) was added for 20 minutes at room temperature on either Biotin or BNHS treated cells on days 1, 2, 4 or 7 to assess the stability of biotin on the cell surface. After addition of SR, the cells were fixed with 4% formaldehyde solution and stained with 100 μL of DAPI solution (1 μg/mL in PBS) after being treated with 100 μL of 0.1% TRITON X solution in PBS. The fluorescent microscopic images of both the BNHS and Biotin treated cells were analyzed by measuring the fluorescent intensity. The stability of the biotin functionality was measured by analyzing the fluorescence intensity of the added rhodamine-streptavidin. The stability of the BNHS functionalization over 7 days was tested compared to biotin without NHS (control) and provided stable biotin groups on the cell membrane over 7 days.

### Viability of the Cells Modified by BNHS

The viability of the cells modified in suspension was tested immediately after modification (0 hours) using Trypan Blue; results indicate that about 69% cells are viable after the modification steps. The controls for the experiment were cells treated with Biotin and cells without any treatment but under similar experimental conditions of suspension and centrifugation. Control cells exhibited ˜80% viability, whereas biotin coated cells exhibited ˜73% viability. This indicates that the BNHS modification does not significantly decrease the viability. The low viability is due the steps of the modification process which decreases the viability.

To assess the effect of the BNHS modification in suspension, the cells were modified in suspension and the viability was tested after 48 hours along with same controls. The BNHS modified cells were 80% viable (compared to 90% viable cell and 85% viable biotin controls) after 48 hours which indicates that the modification does not induce any substantial toxic effect on the cells.

### Adhesion of Modified Cell

Cell adhesion was analyzed by measuring the number of adherent cells on tissue culture wells of 96 well plates after biotinylation of the cells. The cells were biotinylated in suspension as described above. After streptavidin conjugation, the cells were washed and re-suspended in cell media. The cells were counted in a hemocytometer and approximately 5000 cells were placed in each well of 96 well plates for 10, 30 and 90 minutes. After which the non-adhered cells were removed by rinsing with twice with PBS followed by fixing the adhered cells and staining them with toluidine blue solution. The adhered cells were counted in 6 fields of 3 wells at 10× magnification to determine the number of adherent cells.

The adhesion of cells modified in suspension was tested immediately after 10, 30 and 90 min., which shows that about 25% of the modified cells adhere in the first 10 minutes, followed by approximately 40% in 30 minutes, and approximately 68% in 90 minutes. The lower adherence of the cells are attributed to the modification of the cells over the entire cell surface due to which the cells lose a fraction of adhesion molecules. Control cells treated under similar experimental conditions of suspension and centrifugation, show around 36%, 60% and 75% adhesion in 10, 30 and 90 min., respectively. This indicates that the lower adherent characteristics of the cells in not primarily due to the modification by BNHS but also is dependent on the steps of the modification.

### Proliferation Assessment of the Modified Cells

The proliferation of cells modified in suspension was tested for 8 days. After the cells were modified in the suspension by BNHS, approximately 50,000 cells were placed in a T25 flask and counted over the 8 day time period. The growth curve shows proliferation eventually leading to confluent monolayering. Control cells treated under similar experimental conditions of suspension and centrifugation show that the cell modification in suspension does not compromise the proliferative capacity of the modified cells.

Rolling Characteristics of hMSC Modified in Suspension

The hMSC modified in suspension with biotinylated SLeX shows significantly lower velocity than unmodified cells (PBS treated). The velocity of the hMSCs modified by BNHS in suspension was flown through the flow chamber at a shear rate of 0.37 dyne/cm² and cells were rolling at a velocity of 0.55 μm/sec. Compared to the modified cells the controls, where the cells were not modified, moved at a velocity of 75 μm/sec. This shows that introducing SLex through biotin-streptavidin conjugation using BNHS in suspension mode induces to roll the cells more effectively on P-selectin coated surfaces.

The effect of flow rates and thus different shear stress on the rolling characteristics of the modified cells was assessed. The effect of shear stress on the velocity of the hMSCs modified by BNHS in suspension indicates that an increase in shear stress increases the velocity. By changing the shear stress from 0.37 dyne/cm² to 1.8 dyne/cm², an increase in velocity from 0.55 μm/sec to 1.5 μm/sec is observed. This shows that at higher shear stress levels, the modified cells exhibit rolling characteristics with lower velocities, which are comparable to the naturally rolling cells. The effect of shear stress on the flux of the hMSCs modified by BNHS in suspension indicates that an increase in shear stress decreases the flux. By changing the velocity from 0.37 dyne/cm² to 1.8 dyne/cm², a decrease in flux from 155 cells/(mm²×sec) to 118 cells/mm²·sec is observed (compare to unmodified cells, which exhibit a flux of 20 cells/(mm²×sec)). This shows that at higher shear stress levels, the modified cells are able to adhere to the P-selectin surfaces.

### Example 4

### Modification of hMSCS by PLGA Particle Using Biotin-Streptavidin Conjugation

### Preparation of Streptavidin Conjugated PLGA Particle

Carboxylic acid terminated 1 μm PLGA particles were fabricated using standard emulsion-solvent evaporation techniques. Streptavidin was covalently attached to the carboxylic acid group of the PLGA particle using standard carbodiimide coupling technique. Covalently conjugated streptavidin to PLGA (Strep-PLGA particles) particles were washed thrice to remove physically adsorbed streptavidin from the surface of the PLGA particle.

Conjugation of Strep-PLGA Particles to hMSC Surface

hMSCs were biotinylated with 1 mM sulfo-NHS-biotin (BNHS) at room temperature in PBS followed by conjugation of Streptavidin conjugated PLGA (Strep-PLGA) particles. Two negative controls were used for the experiments. In one set of controls Strep-PLGA was added to unmodified cells, i.e. the cells were not biotinylated. In a second set of controls, the cells were biotinylated but unmodified PLGA particles i.e. carboxylic acid terminated PLGA particles were added.

Images of cells with strep-PLGA particles (and the controls) were counted and indicate that the cells with the highest average particles per cell were those cells modified using BNHS/Strep/PGLA. Results shows that Strep-PLGA beads attach to biotinylated hMSCs through specific biotin-streptavidin interactions. There is a significant difference between the experimental group (BNHS+Strep-PLGA) and the negative control groups (Strep-PLGA and BNHS-PLGA). A significantly higher number of strep-PLGA particles attached to the covalently biotinylated hMSC, which indicates that the particles specifically attach to the cells.

Viability of hMSC Modified by Strep-PLGA Particles

The viability of the cells was measured using trypan blue exclusion. Briefly, cells were plated into 12 well plates, left to adhere over night and the cells were treated with BNHS followed by strep-PLGA particle as described above. The number of floating cells observed in the different wells were low and comparable between groups. After rinsing, the cells were detached from the well using 200 μL cell dissociation solution. To it 300 μL of media was added and the total 500 μL of the cell suspension was collected in an Eppendorf tube. From this, 10 μL of cell suspension was diluted to 1:1 by using 4% trypan blue solution and cells were counted in a hemocytometer to determine the number of viable (unstained) and nonviable (blue-stained) cells. The results reported as the mean from triplicate experiments were determined and no change in viability was detected among the groups. The controls for this experiment included cells with no treatment (but PBS was added during the experiment and kept in room temperature). To test the viability after 48 hours, the modified cells were incubated for 48 hours at 37° C. and 5% CO₂. The media was then aspirated and the cells were detached from the well using cell dissociation solution. The viability was tested in the same manner as previously described herein.

Adherence of hMSC Modified by Strep-PLGA Particles

Cell adhesion was analyzed by measuring the number of adherent cells on tissue culture wells of 96 well plates, after biotinylation of the cells. 80% confluent cells in a T25 flask were biotinylated as described above. After strep-PLGA conjugation, the cells were washed and detached from the flask using cell dissociation solution. The cells were counted in a hemocytometer and approximately 5000 cells were placed in each well of a 96 well plate for 10, and 90 minutes. After which, the non-adhered cells were removed by rinsing with twice with PBS, followed by fixing the adhered cells and staining them with toluidine blue solution. The adhered cells were counted in 6 fields of 3 wells at 10× magnification to determine the number of adherent cells. The percentage of adherent cells was calculated based on the initial seeding density of the cells. A control for this experiment included cells with no treatment.

Proliferation Assay of hMSC Modified by Strep-PLGA Particles

The proliferation assay of the cells was analyzed by measuring the number of cells on a T25 flask. 80% confluent cells in a T25 flask were treated with BNHS followed by treatment with strep-PLGA particles solution for the 20 minute time period. Cells were washed and detached from the flask using cell dissociation solution. The cells were counted in a hemocytometer and approximately 50,000 cells were placed in 3 T25 flasks. The number of cells was counted on days 1, 2, 4, 6 and 8 in 10 fields of 3 flasks in 10× magnification after the seeding to assess the number of cells. Control for this experiment is cells treated with PBS.

### Rolling of Strep-PLGA Modified Cells on P-Selectin Surface

The modification of the cells was performed by treating 80% confluent cells in T25 flask. The flask was treated with BNHS followed by treatment with strept-PLGA particles. Then the cells were washed twice with PBS and 1 mL of 4 μg/mL SLex (in PBS, pH 7.4 without Ca/Mg) was added at room temperature for 20 minutes. Cells were detached from the flask using cell dissociation solution and re-dispersed in culture media after centrifugation. The cell concentration for the flow chamber experiment was typically 105 cell/ml. The rolling characteristics of the cells were assessed by a rectangular parallel plate flow chamber experiments with 127 μm gasket thickness and a length of 6 cm. P-selectin immobilization on a glass surface was performed by incubating 700 μL of P-selectin solution (5 μg/mL) on one glass slide for 18 hours and flow experiments were performed by placing the chamber on the glass slides. The flow rate of 20 μL/min corresponding to a wall shear stress 0.366 dyne/cm² was used. To monitor rolling of the cells, the phase contrast microscopy was used, images were recorded in a 10× field and were captured manually approximately every 10 seconds. The velocity of the cells was calculated by measuring the distance of the moving cells over a 10 second time period.

The rolling characteristics of the cell-particle conjugates were measured by determining rolling velocities and flux in both bright field and fluorescence mode (as the PLGA particles were fluorescent) at a shear stress of 0.366 dyne/cm². The velocity of the cell and/or particle conjugates show that following attachment of a particle to a cell, the velocity is slightly higher. This indicates that particles are causing a decrease in the velocity due to steric hindrance However, the velocity of the strep-PLGA modified cells is significantly lower than the velocity (−70 μm/sec) of the unmodified cells, which indicates that the cells specifically interact with P-selectin and thus exhibit rolling based adhesion.

Table 1 shows a list of acyl groups that can be employed in the methods described herein.

TABLE 1

| Carbon Number | | |
|---|---|---|
| (# C: # double bonds | 1-Acyl | 2-Acyl |
| 14:0-16:0 | Myristoyl | Palmitoyl |
| 14:0-18:0 | Myristoyl | Stearoyl |
| 16:0-14:0 | Palmitoyl | Myristoyl |
| 16:0-18:0 | Palmitoyl | Stearoyl |
| 16:0-18:1 | Palmitoyl | Oleoyl |
| 16:0-18:2 | Palmitoyl | Linoleoyl |
| 16:0-20:4 | Palmitoyl | Arachidonoyl |
| 16:0-22:6 | Palmitoyl | Docosahexaenoyl |
| 18:0-14:0 | Stearoyl | Myristoyl |
| 18:0-16:0 | Stearoyl | Palmitoyl |
| 18:0-18:1 | Stearoyl | Oleoyl |
| 18:0-18:2 | Stearoyl | Linoleoyl |
| 18:0-20:4 | Stearoyl | Arachidonoyl |
| 18:0-22:6 | Stearoyl | Docosahexaenoyl |
| 18:1-14:0 | Oleoyl | Myristoyl |
| 18:1-16:0 | Oleoyl | Palmitoyl |
| 18:1-18:0 | Oleoyl | Stearoyl |

### Example 5

### Modification of hMSCS by PLGA Particle Using Non-Covalent Methods

### Preparation of Negatively Charged PLGA Particle

Carboxylic acid terminated 1 μm PLGA particles were fabricated using standard emulsion-solvent evaporation techniques from PLGA functionally terminated by negatively charged carboxylic acid.

### Preparation of Positively Charged PLGA Particle

Negatively charged PLGA particles were incubated with positively charged poly-L-lysine at an excess concentration (0.5 mg/mL in PBS) at room temperature for 2 hours. The charged interaction between the negative charge on the PLGA particle surface and the positive charge of poly-L-lysine results on the excess positive charge on the particle surface.

### Preparation of Lipid-Coated PLGA Particle

Carboxylic acid terminated 1 μm PLGA particles were fabricated using standard emulsion-solvent evaporation techniques. Streptavidin was covalently attached to the carboxylic acid group of the PLGA particle using standard carbodiimide coupling technique. Covalently conjugated streptavidin to PLGA (Strep-PLGA particles) particles were washed thrice with PBS to remove physically adsorbed streptavidin from the surface of the PLGA particle. The streptavidin conjugated PLGA particles were incubated in biotinylated lipid, 1,2-Dipalmitoyl-sn-Glycero-3-Phosphoethanolamine-N-(Biotinyl) (Sodium Salt), (0.1 mg/mL in PBS) solution for 2 hours at room temperature and subsequently washed with PBS to remove excess and physically adsorbed lipid molecules.

### Preparation of Antibody-Coated PLGA Particle

Carboxylic acid terminated 1 μm PLGA particles were fabricated using standard emulsion-solvent evaporation techniques. Streptavidin was covalently attached to the carboxylic acid group of the PLGA particle using standard carbodiimide coupling technique. Covalently conjugated streptavidin to PLGA (Strep-PLGA particles) particles were washed with PBS thrice to remove physically adsorbed streptavidin from the surface of the PLGA particles. The streptavidin conjugated PLGA particles were incubated in biotinylated CD90 antibody, (0.01 mg/mL in PBS) solution for 2 hours at room temperature and subsequently washed with PBS to remove excess and physically adsorbed antibodies.

Non Covalent Conjugation of PLGA Particle to hMSCs

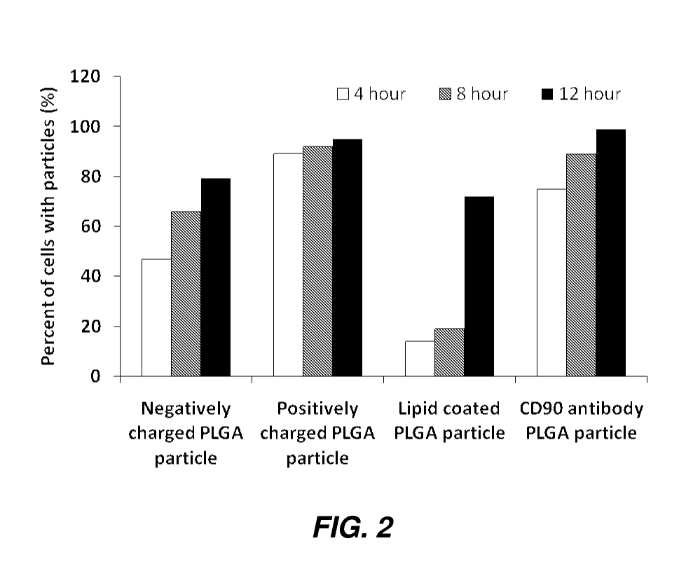

PLGA particles with various surface characteristics were added to the adherent hMSCs on tissue culture plates and washed subsequently to remove non-adhered particles from the cell. For characterization, after 4 hours, 8 hours and 12 hours of incubation, the cells were washed, fixed with 4% formaldehyde, and then stained with propidium iodide (PI) (10 μg/mL in PBS) for 10 minutes and washed twice with PBS. The cell-particle conjugates were imaged using fluorescence microscopy for PI stained cells and DiO encapsulated PLGA particles. The number of cells conjugated with particles was calculated for four different surface characteristics and is expressed as the percentage of cells having particles as shown in FIG. 2. The results showed that particles with all surface functionalities bound to the MSCs, where positively charged particles and CD90 antibody coated particles bound most effectively while lipid coated particles and negatively charged particles attached least effectively up to 8 hours. The attachment of lipid-coated particles increased up to 70% after 12 hours of incubation.

Uptake of PLGA Particle Conjugated to hMSCs by Non-Covalent Methods

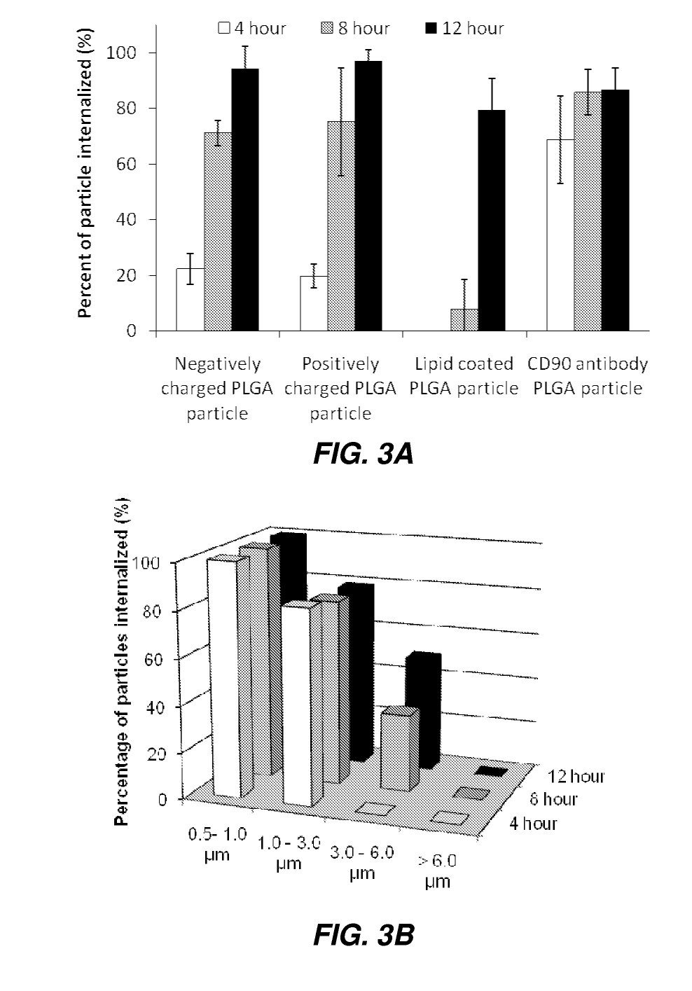

The stability of PLGA particles conjugated to the hMSC surface was visualized through laser confocal microscopy by examining the Z-stack images of the cells (red, PI stained) particle (DiO encapsulated, green). Confocal microscopic images were taken after 4 hours, 8 hours, and 12 hours of incubation. Z-stack confocal images from y-z, x-z and x-y planes were analyzed to assess the internalization of the particles. Particles in the field of view but not associated with the cells were not considered. The percent of internalization was calculated from the total number of particles conjugated to the cell and the number of particles internalized and is shown in FIG. 3A. CD90 antibody coated particles were most effectively internalized whereas no lipid coated particles were internalized after 4 or 8 hours. The internalization of lipid coated PLGA particles increased to 80% after 12 hours of incubation. For negatively charged particle and positively charged particles, the particle uptake by cells increased from 20% to 60% from 4 to 8 hours, respectively. This indicates that lipids functionalized onto the cell surface are stabile for up to 8 hours. The effect of particle size also influences the internalization of the particles as shown in FIG. 3B. The results indicate that for antibody coated PLGA particles lower than 3 μm are internalized with greater efficiency compared to larger particles. 50% of the particles greater than 3 μm are internalized at 12 hours indicating that larger sized particles are stabilized on the cell surface at lower time.

Analysis of Negatively Charged PLGA Particles Conjugated to hMSCs During Culture and the Effect of Trypsinization on the Stability of the Particles

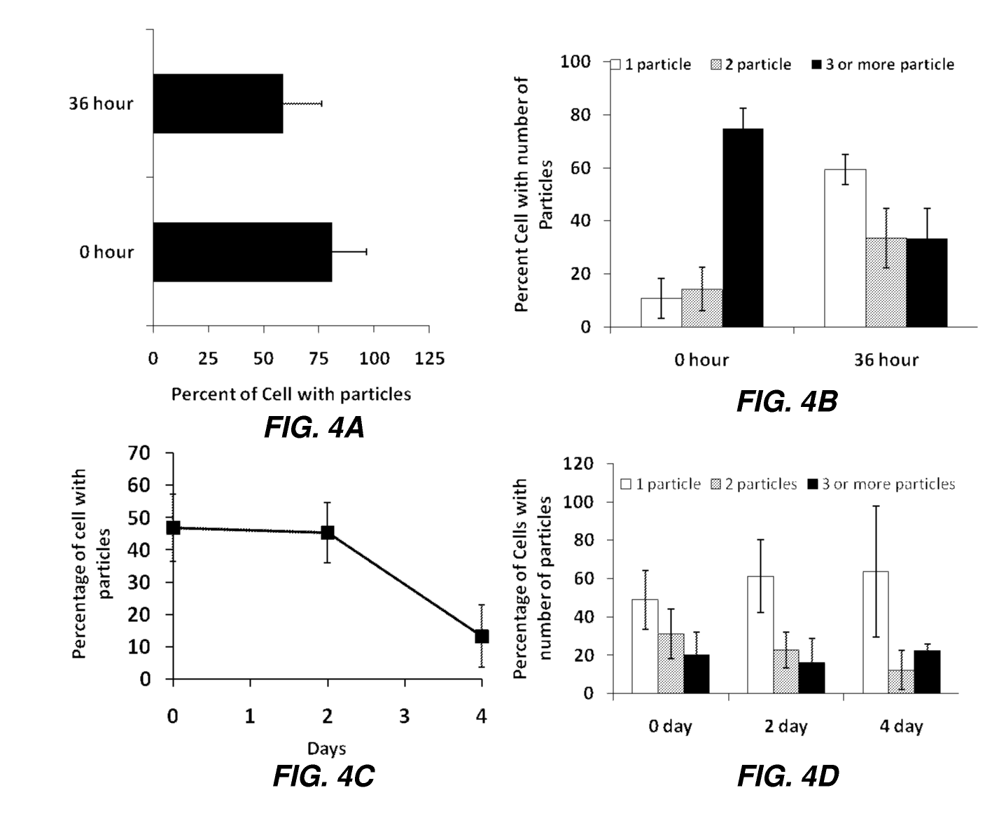

The conjugation of the negatively charged PLGA particles to the cells shows that 80% of the cells contain conjugated particles immediately after conjugation and 60% of the cells contain conjugated to particles after 36 hours of incubation (FIG. 4A, 4B). This change in numbers of particles per cell is likely due to cell proliferation. After incubating the cells with negatively charged PLGA particles for 36 hours, the cell were trypsinized using 1× trypsin-EDTA solution and then washed with phosphate buffer saline (PBS without Ca/Mg, pH 7.4) to remove media and trypsin and were plated on 6 well plates. FIG. 4C shows the percent of cells conjugated with negatively charged PLGA particles after trypsinization (day 0) and on day 2 and day 4. This indicates that the particles are attached to the cells after trypsinization and shows that trypsinization of cells with particles does not impact the stability of the particles on the cell surface. The percentage of cells conjugated to 1, 2 and 3 or more particles after trypsinization is shown in FIG. 4D.

Induction of Osteogenic Differentiation by Negatively Charged PLGA Particle Conjugated to hMSCs

Dexamethasone (osteogenic differentiation factor for hMSCs) was encapsulated within the PLGA particles. hMSCs were incubated with dexamethasone containing PLGA particle in presence of Ascorbic acid and β-glycerol phosphate in the media and osteogenesis was assessed by alkaline phosphatase and von-Kossa staining after 21 days. (80% of cells contained Dexamethasone containing PLGA particles with more than 70% cells having 3 or more particles). Negative control groups include hMSC with PLGA particle attached without dexamethasone with ascorbic acid and β-glycerol phosphate in expansion media; hMSC with ascorbic acid and β-glycerol phosphate in expansion media without particles and hMSC in expansion media. Positive controls include hMSC with dexamethasone, ascorbic acid and β-glycerol phosphate in expansion media. The experimental group i.e. dexamethasone containing PLGA particles in the presence of Ascorbic acid and β-glycerol phosphate shows significant positive osteogenic staining compared to control groups indicating that dexamethasone encapsulated within the PLGA can induce the osteogenic differentiation of the cells. This indicates that PLGA particles non-covalently attached to hMSCs can specifically induce osteogenic differentiation through the factors encapsulated within the particles.

The alkaline phosphatase assay was performed by aspirating the medium and washing the cells with distilled water. The cells were fixed with 3.7% formaldehyde solution for 15 min at room temperature and then washed twice with distilled water. To this 0.06% Red Violet LB salt solution in Tris HCl and distilled water containing (DMF and Naphthol AS MX-PO4) was added. The plates were incubated for 45 minutes and then the wells were rinsed 3 times with distilled water. After the alkaline phosphate assay, the cells were incubated for 30 minutes with 2.5% silver nitrate solution in dark for von Kossa staining. After the incubation, the wells were rinsed 3 times with distilled water.

In Vivo Transendothelial Migration Negatively Charged PLGA Particles Attached to Human Mesenchymal Stem Cells (hMSCs)

Negatively charged fluorescent PLGA particles (DiO encapsulated, average size 1-2 μm) were attached to hMSCs in a 90% confluent T75 flask for 24 hours prior to the experiment. The cells were trypsinized and washed once with PBS to remove trypsin. The cells were fluorescently labeled with DiD. Typically 500,000 cells (DiD labeled hMSCs modified with DiO particles) in 200 μL of PBS were injected into the tail vein. A control for this experiment was injection of a similar number of unmodified hMSCs. Intravital in vivo confocal microscopy was used to acquire Z-stack images (to ensure no overlap) to visualize the cells in the marrow within the skull after 24 hours after injection. The vessels in marrow were fluorescently labeled prior to imaging. Extravasation of the hMSCs with particles through the bone marrow endothelium was observed using co-localization of red fluorescence (from DiD labeled hMSCs) and green fluorescence (from DiO encapsulated particles), indicating that negatively charged PLGA particle attached to hMSCs can transmigrate through the endothelium.

### Example 6

### Modification of hMSCS by Direct Biotinylation of Cells Followed by Addition of PLGA Particles Containing a Biotin-Streptavidin Bridge

### Preparation of Streptavidin Conjugated PLGA Particle

Carboxylic acid terminated 1 μm PLGA particles were fabricated using standard emulsion-solvent evaporation techniques. Streptavidin was covalently attached to the carboxylic acid group of the PLGA particle using standard carbodiimide coupling technique. Covalently conjugated streptavidin to PLGA (Strep-PLGA particles) particles were washed thrice to remove physically adsorbed streptavidin from the surface of the PLGA particle.

Conjugation of Strep-PLGA Particles to hMSC Surface

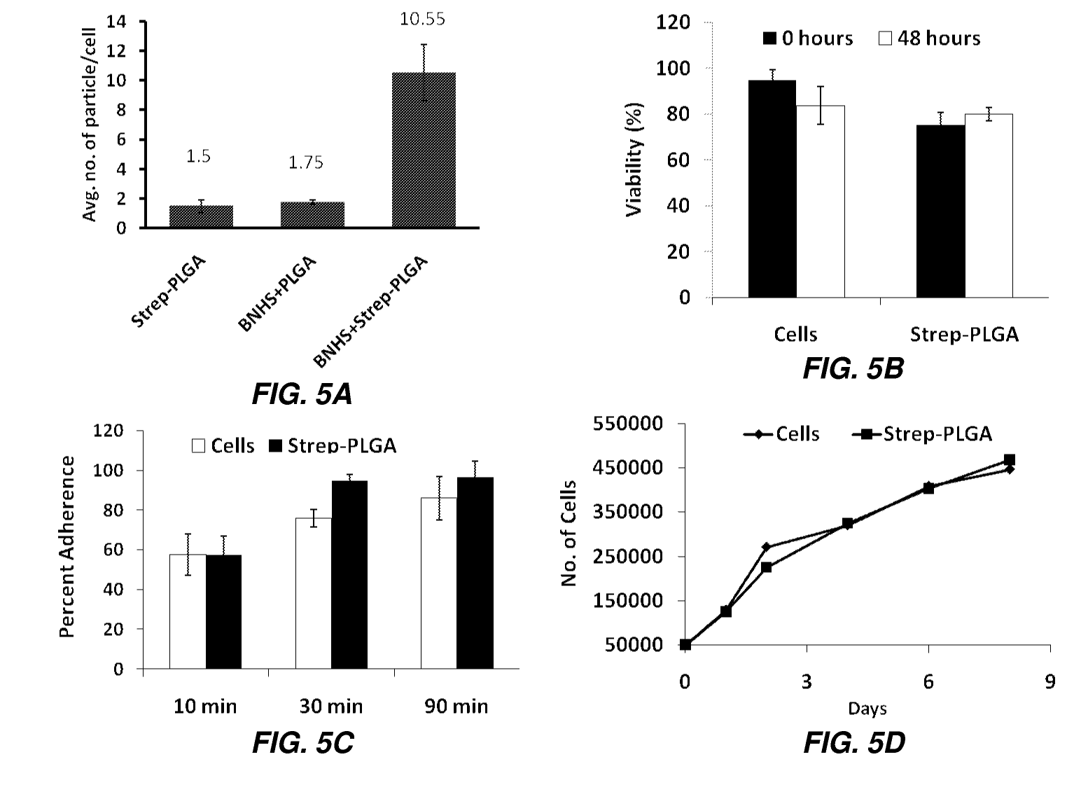

hMSCs were biotinylated with 1 mM sulfo-NHS-biotin (BNHS) at room temperature in PBS followed by conjugation of Streptavidin conjugated PLGA (Strep-PLGA) particles as shown in FIG. 5A. Two negative controls were used for the experiments. In one set of controls Strep-PLGA was added to unmodified cells, i.e. the cells were not biotinylated. In a second set of controls, the cells were biotinylated but unmodified PLGA particles i.e. carboxylic acid terminated PLGA particles were added.

Results (FIG. 5A) show that Strep-PLGA beads attach to biotinylated hMSCs through specific biotin-streptavidin interactions. There is a significant difference between the experimental group (BNHS+Strep-PLGA) and the negative control groups (Strep-PLGA and BNHS-PLGA). A significantly higher number of strep-PLGA particles attached to the covalently biotinylated hMSC, which indicates that the particles specifically attach to the cells.

Viability of hMSC Modified by Strep-PLGA Particles

The viability of the cells was measured using trypan blue exclusion. Briefly, cells were plated into 12 well plates, left to adhere over night and the cells were treated with BNHS followed by strep-PLGA particle as described above. The number of floating cells observed in the different wells were low and comparable between groups. After rinsing, the cells were detached from the well using 200 μL cell dissociation solution. To it 300 μL of media was added and the total 500 μL of the cell suspension was collected in an Eppendorf tube. From this, 10 μL of cell suspension was diluted to 1:1 by using 4% trypan blue solution and cells were counted in a hemocytometer to determine the number of viable (unstained) and nonviable (blue-stained) cells. The results reported as the mean from triplicate experiments are shown in FIG. 5B. The controls for this experiment included cells with no treatment (but PBS was added during the experiment and kept in room temperature). To test the viability after 48 hours, the modified cells were incubated for 48 hours at 37° C. and 5% CO₂. The media was then aspirated and the cells were detached from the well using cell dissociation solution. The viability was tested in the same manner as previously described herein.

Kinetics of Adhesion for hMSC Modified by Strep-PLGA Particles

Cell adhesion was analyzed by measuring the number of adherent cells on tissue culture wells of 96 well plates, after biotinylation of the cells. 80% confluent cells in a T25 flask were biotinylated as described above. After strep-PLGA conjugation, the cells were washed and detached from the flask using cell dissociation solution. The cells were counted in a hemocytometer and approximately 5000 cells were placed in each well of a 96 well plate for 10, and 90 minutes. After which, the non-adhered cells were removed by rinsing twice with PBS, followed by fixing the adhered cells and staining them with toluidine blue solution. The adhered cells were counted in 6 fields of 3 wells at 10× magnification to determine the number of adherent cells. The percentage of adherent cells was calculated based on the initial seeding density of the cells and the results are shown in FIG. 5C. A control for this experiment included cells with no treatment.

Proliferation Assay of hMSC Modified by Strep-PLGA Particles

The proliferation assay of the cells was analyzed by measuring the number of cells on a T25 flask. 80% confluent cells in a T25 flask were treated with BNHS followed by treatment with strep-PLGA particles solution for the 20 minute time period. Cells were washed and detached from the flask using cell dissociation solution. The cells were counted in a hemocytometer and approximately 50,000 cells were placed in 3 T25 flasks. The number of cells was counted on days 1, 2, 4, 6 and 8 in 10 fields of 3 flasks in 10× magnification after the seeding to assess the number of cells. The results are shown in FIG. 5D. Control for this experiment is cells treated with PBS.

Differentiation Potential of the Strep-PLGA Particles Modified hMSCs

In vitro osteogenic and adipogenic differentiation potential of cells modified by Strep-PLGA conjugation was assessed, as described below.

### Osteogenic Differentiation

hMSCs were seeded in 4 wells of a 24 well plate and were cultured in a cell expansion media until reaching 90% confluence. The modification of the cells was performed by a two step method. Typically the cells were biotinylated with 1 mM BNHS followed by conjugation with strep-PLGA particles. Hereafter, the osteogenic differentiation was induced by culturing the cells for 21 days in osteogenic induction media (from Lonza—hMSCs Osteogenic Single Quote kit) containing dexamethasone, β-glycerophosphate, L-ascorbic acid-2-phosphate, and a-MEM. As a positive control, an equal number of wells with untreated (un-modified) cells were maintained in osteogenic induction medium. The media in both groups was changed every 3 days. Osteogenesis was evaluated by alkaline phosphatase and von Kossa staining.

The alkaline phosphatase assay was performed by aspirating the medium and washing the cells with distilled water. The cells were fixed with 3.7% formaldehyde solution for 15 min at room temperature and then washed twice with distilled water. To it 0.06% Red Violet LB salt solution in Tris HCl and distilled water containing (DMF and Naphthol AS MX-PO4) was added. The plates were incubated for 45 minutes and then the wells were rinsed 3 times with distilled water.

The osteogenic differentiation potential of cells modified by strep-PLGA particles was observed by alkaline phosphatase staining for the modified cells. The particle modified cells showed positive staining and comparable results with unmodified cells. This indicates that the particle modification of the cells likely does not interfere with the osteogenic potential of the cells and modified cells can differentiate into their osteogenic lineages.

### Adipogenic Differentiation

hMSCs were seeded in 4 wells of a 24 well plate and were cultured in a cell expansion media until reaching 100% confluence. The modification of the cells was performed by a two step method. Typically the cells were biotinylated with 1 mM BNHS followed by conjugation with streptavidin (50 μg/mL) in PBS at room temperature. Thereafter, the adipogenic differentiation was induced by culturing the cells for 23 days in adipogenic induction media (from Lonza—hMSCs Adipogenic Single Quote kit containing h-Insulin (recombinant), L-Glutamine, Dexamethasone, Indomethacin, IBMX (3-isobuty-1-methyl-xanthine), Pen/Strep) and adipogenic maintenance media (from Lonza—hMSCs Adipoogenic Single Quote kit containing h-Insulin (recombinant), L-Glutamine, Pen/Strep). As a positive control, an equal number of wells with untreated (un-modified) cells were maintained in adipogenic induction medium. The media in both groups was changed every 3 days according to Lonza protocol in a periodical exposure of induction and maintenance media. Adipogenesis was evaluated by Oil Red O staining.

The Oil Red O staining protocol used is as follows: aspirate all of the media off of the cells, wash once with PBS, replace the PBS with 3.7% formaldehyde for 30 min at room temperature to fix the cells, replace the formaldehyde with distilled water for a few minutes, replace isopropanol with Oil Red O working solution (made by diluting 30 ml of 0.5% isopropanol solution of Oil Red with 20 ml distilled water), after 5 minutes the Oil red O solution was washed twice with distilled water. One ml of hematoxylin (Sigma-Aldrich) was added to the well for 1 minute before being aspirated and the wells were washed with distilled water. The wells were viewed using an inverted phase contrast microscope. Lipids appear red and nuclei appear blue.

The adipogenic differentiation potential of cells modified by Strep-PLGA particles was observed by Oil Red O and hemotoxylin staining. The particle modified cells showed positive staining and comparable results with unmodified cells. This indicates that the particle modification of the cells does not likely interfere with the adipogenic potential of the cells and modified cells can differentiate into their adipogenic lineages.

Rolling of Biotinylated Cells Modified with Strep-PLGA Particles where the Cells and Particles are Functionalized with SLeX

The modification of the cells were carried out by biotinylating the cells with BNHS followed by conjugation of strep-PLGA particle in presence of streptavidin solution. The strep-PLGA particle conjugated cells were subsequently treated to conjugate biotinylated SLeX both on the cell surface and on the particle surface. The presence of SLeX on particle surface and on cell surface can induce rolling interaction on P-selectin surface. The modification of the cells was performed by treating 80% confluent cells in T25 flask. The flask was treated with BNHS followed by treatment with strep-PLGA particles in presence of streptavidin solution (50 μg/mL in PBS without Ca/Mg). Then the cells were washed twice with PBS and 1 mL of 4 μg/mL SLex (in PBS, pH 7.4 without Ca/Mg) was added at room temperature for 20 minutes. Cells were detached from the flask using cell dissociation solution and re-dispersed in culture media after centrifugation. The cell concentration for the flow chamber experiment was typically 10⁵ cells/ml. The rolling characteristics of the cells were assessed by a rectangular parallel plate flow chamber experiments with 127 μm gasket thickness and a length of 6 cm. P-selectin immobilization on a glass surface was performed by incubating 700 μL of P-selectin solution (5 μg/mL) on one glass slide for 18 hours and flow experiments were performed by placing the chamber on the glass slides. The flow rate of 20 μL/min corresponding to a wall shear stress 0.366 dyne/cm² was used. To monitor rolling of the cells, the phase contrast microscopy was used, images were recorded in a 10× field and were captured manually approximately every 10 seconds. The velocity of the cells was calculated by measuring the distance of the moving cells over a 10 second time period.

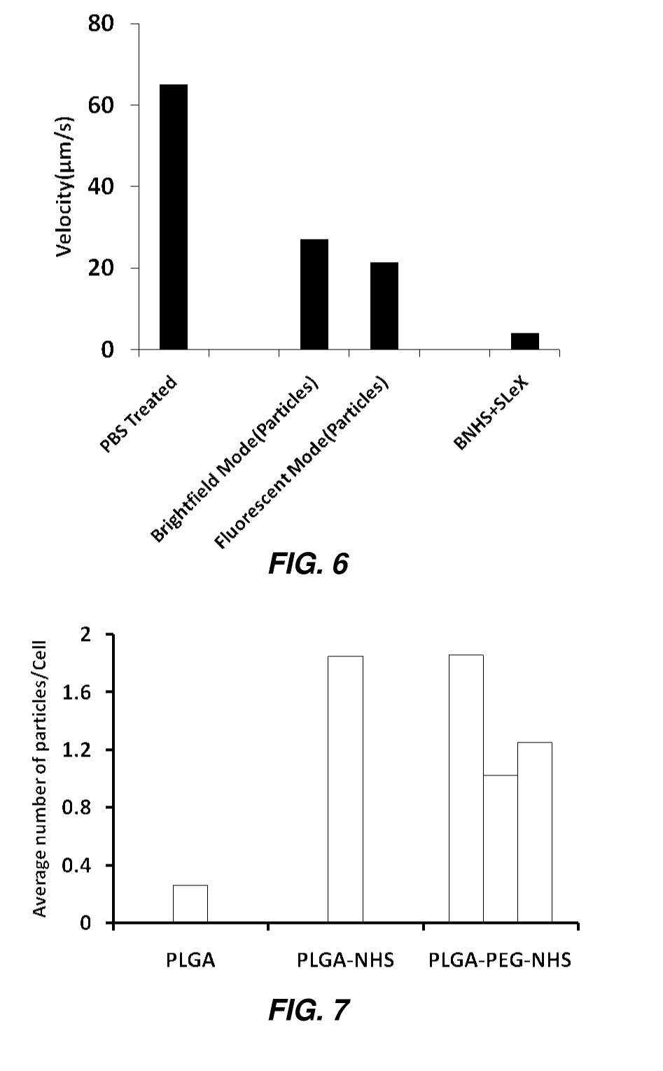

The rolling characteristics of the cell-particle conjugates were measured by determining rolling velocities and flux in both bright field and fluorescence mode (as the PLGA particles were fluorescent) as shown in FIG. 6 at a shear stress of 0.366 dyne/cm². The velocity of the cell and/or particle conjugates indicates that attachment of particles still permits an adhesion based rolling response.

Induction of Osteogenic Differentiation by Strep-PLGA Particle Conjugated to hMSCs

The Strep-PLGA particle conjugated to hMSC through biotin-streptavidin can specifically control the fate of the cells. Specifically dexamethasone (osteogenic differentiation factor for hMSCs) was encapsulated within the PLGA particle. The dexamethasone containing PLGA particles were conjugated to streptavidin through carbodiimide coupling. hMSCs were bionylated with 1 mM sulfo-NHS-biotin (BNHS) at room temperature in PBS followed by conjugation of Streptavidin conjugated PLGA (Strep-PLGA) particles containing dexamethasone. hMSCs conjugated to dexamethasone containing PLGA particle through biotin-streptavidin were cultured in presence of Ascorbic acid and β-glycerol phosphate in the media and the osteogenic differentiation was assessed by alkaline phosphatase and von-Kossa staining after 21 days. Negative control groups include hMSC with PLGA particle attached without dexamethasone with ascorbic acid and β-glycerol phosphate in media; hMSC with ascorbic acid and β-glycerol phosphate in media and hMSC in proliferation media. Positive controls include hMSC with dexamethasone, ascorbic acid and β-glycerol phosphate in media. The experimental group i.e. dexamethasone containing PLGA particle in presence of Ascorbic acid and β-glycerol phosphate shows significantly positive osteogenic stain compared to the controls indicating that dexamethasone encapsulated within the PLGA can induce the osteogenic differentiation of the cells. This indicates that PLGA particle covalently attached to hMSCs through biotin-streptavidin can specifically induce osteogenic differentiation of the cells through the factors encapsulated within the particles.

### Example 7

### Modification of hMSCS by PLGA Particle Using n-Hydroxy Succinimide (NHS) Functional Group and with NHS Group Associated with a PEG Linker

Preparation of NHS Functionalized PLGA Particle with and without a PEG Linker

Carboxylic acid terminated 1 μm PLGA particles were fabricated using standard emulsion-solvent evaporation techniques. Carboxylic acid functional group of the PLGA particles were converted to N-hydroxy succinimide (NHS) group through 1-Ethyl-3-(3-dimethylaminopropyl)-carbodiimide (EDC) and NHS coupling. The NHS functionalized PLGA particles were conjugated to PEG through the reaction of bifunctional polyethylene glycol (functionalized with carboxylic acid and primary amine, MW: 7500) and the NHS group of NHS functionalized PLGA particle. The carboxylic end group of the PEG was further functionalized with EDC and NHS.

Conjugation of NHS Functionalized PLGA Particle with and without a PEG Linker to hMSCs

hMSCs were conjugated to NHS functionalized PLGA particle with and without PEG linker through incubation of functionalized particles with hMSCs. FIG. 7 shows that NHS group reacts with the cell surface to conjugate the particles whereas PLGA particles without any functional group do not conjugate to cells. This indicates that through covalent reaction between the cell surface functionalities (amine group on cell surface) and the functional groups of the particles (i.e. NHS group with and without PEG linker) can be used to conjugate a particle on the surface of the cells. The number of PEG linkers on the PLGA particle can be varied by changing the concentration of the bifunctional PEG molecule during the reaction of bifunctional polyethylene glycol (functionalized with carboxylic acid and primary amine) and the NHS group of NHS functionalized PLGA particle. Specifically 1, 0.1 and 0.01 mg/mL of PEG solutions were used for functionalizing the NHS activated PLGA particle. Results in FIG. 7 indicate that by changing the concentration of PEG linkers, the number of PLGA particles (functionalized with PEG and subsequently activated with NHS) conjugated to the cell can be varied. FIG. 7 shows that with increased concentration PEG linker, higher numbers of particles are conjugated to the cells.

Induction of Osteogenic Differentiation by PLGA Particle Conjugated to hMSCs through NHS with PEG Linker

The PLGA particle conjugated to hMSC through NHS group with a PEG linker can specifically control the fate of the cells. Specifically dexamethasone (osteogenic differentiation ingredient for hMSCs) was encapsulated within the PLGA particle. The dexamethasone containing PLGA particles were functionalized with PEG and NHS through carbodiimide coupling. hMSCs conjugated to dexamethasone containing PLGA particle were cultured in the presence of Ascorbic acid and β-glycerol phosphate in the media and osteogenic differentiation was assessed by alkaline phosphatase and von-Kossa staining after 21 days. Negative control groups include hMSC with PLGA particle attached without dexamethasone with ascorbic acid and β-glycerol phosphate in media; hMSC with ascorbic acid and β-glycerol phosphate in media and hMSC in proliferation media. Positive controls include hMSC with dexamethasone, ascorbic acid and β-glycerol phosphate in media. The experimental group i.e. dexamethasone containing PLGA particle in presence of Ascorbic acid and β-glycerol phosphate shows significantly positive osteogenic stain compared to the controls indicating that dexamethasone encapsulated within the PLGA can induce the osteogenic differentiation of the cells. This indicates that PLGA particles covalently attached to hMSCs through NHS functionality with a PEG linker can specifically induce osteogenic differentiation of the cells through the factors encapsulated within the particles.

### Example 8

### In Vivo Experiments with Human Mesenchymal Stem Cells (hMSCs) Modified by Sialyl Lewis X (SLeX)

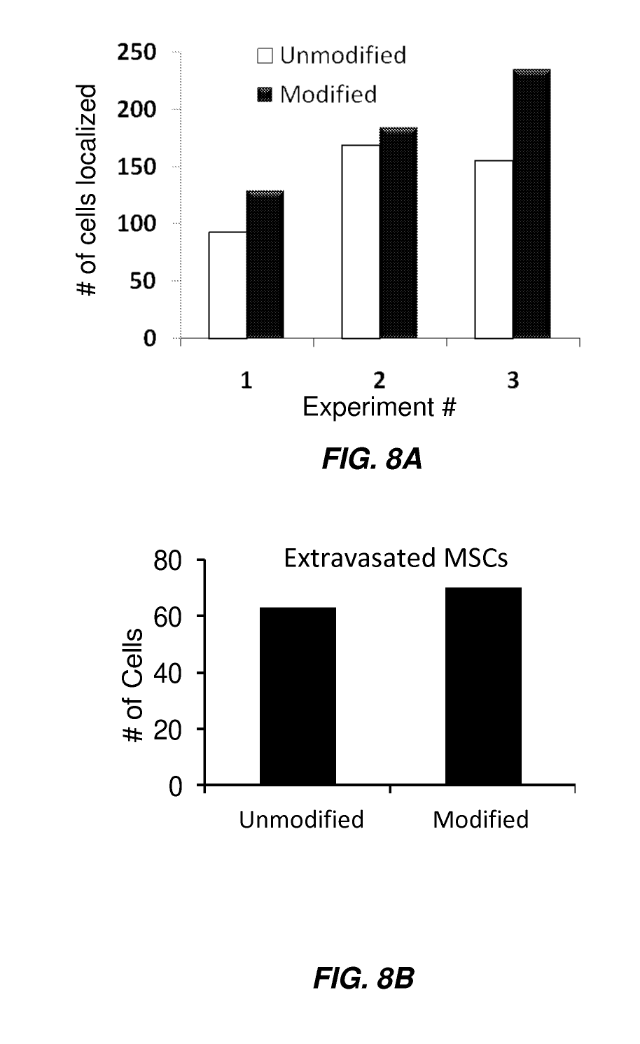

hMSCs modified with SLeX through biotin and streptavidin were injected into mice through tail vein injection. Briefly, the hMSCs were treated with 1 mL of 1 mM BNHS solution followed by 1 mL of 50 μg/mL of streptavidin solution and 1 mL of 5 μg/mL of biotinylated SLeX at room temperature. The modified cells were fluorescently labeled with DiD to image the cells after the injection. Typically 500,000 cells (DiD labeled hMSCs modified with SLeX) in 200 μL of PBS were injected through tail vein. Control for this experiment was unmodified hMSCs injected with similar number. Intravital in vivo confocal microscopy was used to image the cells in the marrow within the skull at 2 hours and 24 hours after injection. The vessels in marrow were fluorescently labeled prior to imaging. FIG. 8A shows the number of unmodified and modified MSCs (MSCs are modified with biotin-N-hydroxy succinimde followed by streptavidin and biotinylated sialyl Lewis X, SLeX) localized in the bone marrow after 2 hours of tail vein injection of cells in three separate experiments #1, 2, 3. Higher numbers of modified MSCs compared to unmodified MSCs were localized to the marrow 2 hours after the injection. FIG. 8B shows the number of extravasated MSCs that transmigrated through the bone marrow endothelium 24 hours after the injection. There was no difference in the transendothelial migration capability between the modified and unmodified MSCs after 24 hour indicating that the covalent modification of the cell surface and attachment of SLeX through a biotin-streptavidin bridge does not impair the transmigration capability of the MSCs.

### Example 9

### Examination of Secretion of Paracrine Factors of Human Mesenchymal Stem Cells (hMSCs) Covalently Modified by Sialyl Lewis X (SLeX) Through a Streptavidin-Biotin Bridge

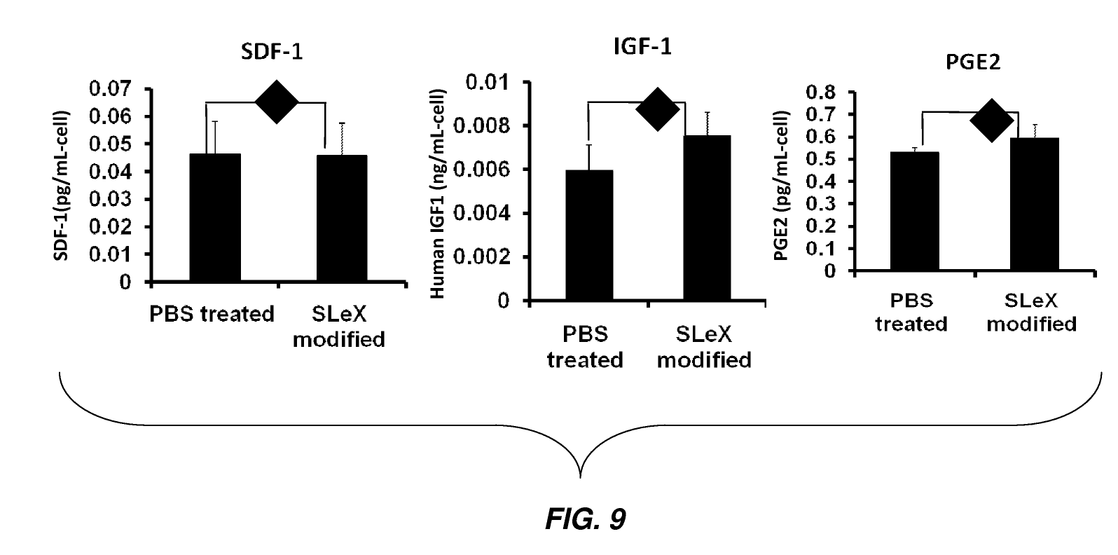

The hMSCs were modified by treating with 1 mL of 1 mM BNHS solution followed by 1 ml of 50 μg/mL of streptavidin solution and 1 mL of 5 μg/mL of biotinylated SLeX at room temperature. After the modification the modified cells were plated into 24 well plates with cell expansion media and incubated at 37° C. for 24 hours. ELISA assays were performed to examine the level of expression of SDF-1, IGF-1 and PGE-2 in the cell culture supernatant. Controls for this experiment include unmodified hMSCs. FIG. 9 shows that the modification of hMSCs with SLeX do not change the level of paracrine factors secreted by the modified hMSCs compared to the unmodified cells at after 24 hours. IGF-1 and PGE2 were not detectable in expansion media containing 15% serum, however, SDF-1 was detected at ˜5% of the amount observed from MSCs after 24 hours.

### Example 10

### Modification of Human Mesenchymal Stem Cells (hMSCs) by P-Selectin Antibody (Ab)

Typically, hMSCs were cultured in a T75 flask up to 90% confluence in hMSC expansion media (15% Fetal Bovine Serum, 1% L-Glutamine, 1% Penn-Strep in a-MEM), after which the cells were trypsinized using 1× trypsin-EDTA solution and then washed with phosphate buffer saline (PBS without Ca/Mg, pH 7.4) to remove media and trypsin. The cell pellet was then dispersed in 1 mL of 1 mM BNHS. After that the cells were centrifuged and spun down to remove the BNHS solution. The cell pellets were washed with PBS twice by resuspending the cells followed by centrifugation. After the centrifugation, the cell pellet was re-suspended in 1 mL of streptavidin solution (50 μg/mL in PBS, pH 7.4 without Ca/Mg). The unconjugated streptavidin was removed by centrifugation. The cell pellet was resuspended in 1 mL of biotinylated P-selectin anti body solution (5 μg/mL in PBS, pH 7.4 without Ca/Mg). The unconjugated antibody was removed by centrifugation.

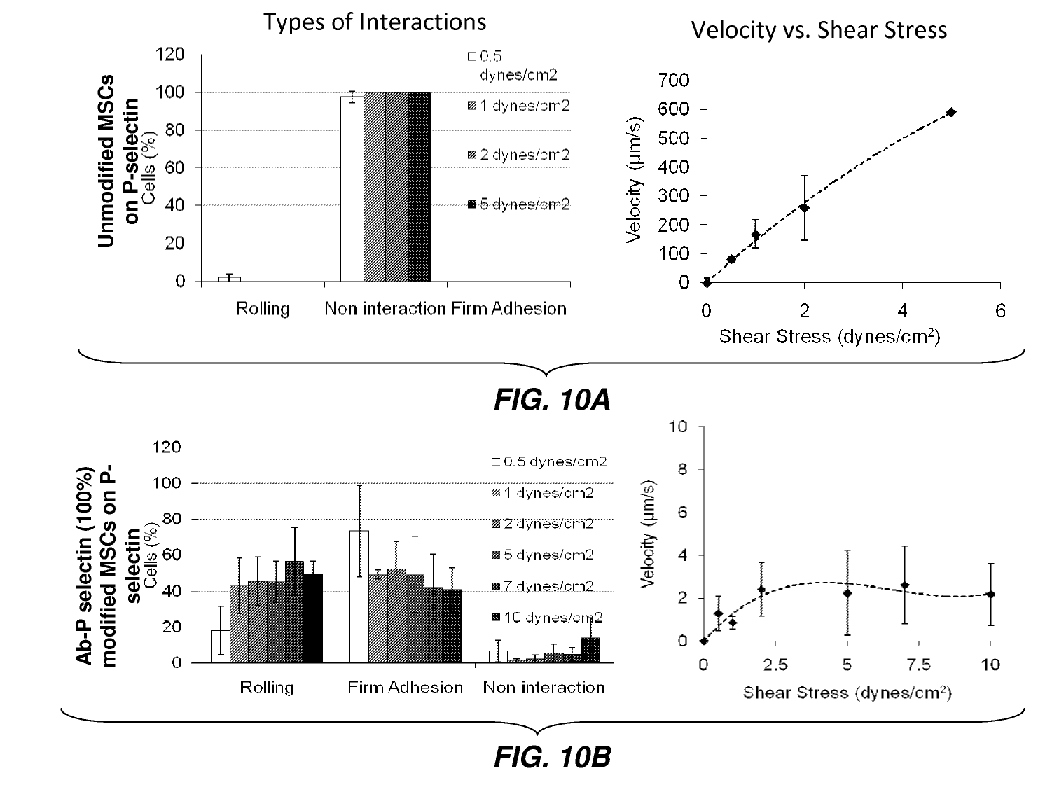

The hMSC modified in suspension with biotinylated P-selectin antibody shows that the modified cells interact with P-selectin coated substrate in a flow chamber assay. The unmodified cells either displayed rolling interaction or firm adhesion in the flow chamber up to 10 dynes/cm² whereas the unmodified cells showed no interaction. This indicates that P-selectin antibody conjugated hMSCs specifically interacts with P-selectin under flow condition (FIG. 10). The velocity of the P-selectin antibody modified cells which displayed rolling interactions were able to roll on the P-selectin surface with an average velocity of 3 μm/sec.

### Example 11

### Quantification of Biotin on Biotinylated Human Mesenchymal Stem Cells (hMSCs) Surface by Biotin-HABA-Avidin Assay

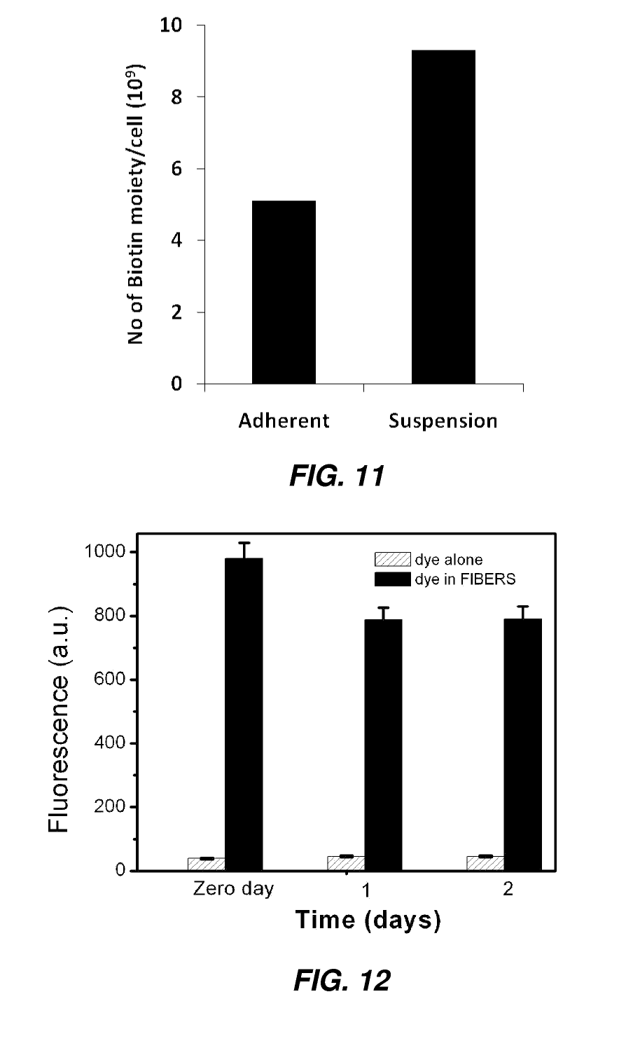

The number of biotin moiety present on the surface of biotinylated hMSCs (biotinylated with BNHS) were quantified by using biotin-HABA-avidin assay according to manufacturer's protocol (Thermo scientific, IN). Specifically two techniques were used to biotinylate the cells: adherent mode and suspension mode. In adherent mode, 80-90% confluent monolayer of hMSCs on T25 flask were treated with 1 mL of 1 mM BNHS for 10 minutes followed by washing with PBS for 3 times. Then cells were detached from the plate using 1 mL of non enzymatic cell dissociation solution and were analyzed according to the biotin-HABA-avidin assay protocol to quantify the number of biotin ligand present on the cell. In suspension mode, a T75 flask up to 90% confluence in hMSC expansion media (15% Fetal Bovine Serum, 1% L-Glutamine, 1% Penn-Strep in a-MEM), were trypsinized using 1× trypsin-EDTA solution and then washed with phosphate buffer saline (PBS without Ca/Mg, pH 7.4) to remove media and trypsin. The cell pellet was then dispersed in 1 mL of 1 mM BNHS. After that the cells were centrifuged and spun down to remove the BNHS solution. The cell pellets were washed with PBS twice by resuspending the cells followed by centrifugation and was analyzed according to the biotin-HABA-avidin assay protocol to quantify the number of biotin ligand present on the cell. FIG. 11 shows that number of biotin present on the surface of hMSCs modified in adherent mode is less than the hMSCs modified in suspension mode. This indicates that only a part of the cell surface undergoes modification in adherent mode as a part of the cell surface is attached to the culture dish whereas in suspension mode the entire cell surface is exposed to modification.

### Example 12

### Modification of hMSCS by Self-Assembled Fibers in an Adherent Mode

### Preparation of Dye Encapsulated Self-Assembled Fibers.

Typically, 4-8 mg of small molecular amphiphile (salicin decanoate) was taken in a screw-capped glass vial, to that 180 μL of PBS and 20 μL DiD (dye) in DMSO were added and capped vial was heated till 60-80° C. until it dissolves, then left untouched at room temperature for cooling. When vial reached room temperature samples were formed hydrogels. Subsequently, hydrogels were diluted using 300 μL PBS and transferred into an Eppendorf and centrifuged with 10,000 rpm for 7 min. Supernatant was removed to wash unbound dye, and fibers were redispersed in 1 mL PBS.

Loading of Dye Encapsulated Self-Assembled Fibers on hMSCs.

hMSCs were seeded in 4 wells of a 24 well plate and were cultured in a cell expansion media until reaching 90% confluence. Dye encapsulated self-assembled fibers were added to the adherent hMSCs on tissue culture plates and incubated for 30 min and washed subsequently to remove non-adhered self-assembled fibers from the cell. The cell-fiber conjugates were imaged using fluorescence microscopy where DiD give red emission. Quantifying the dye on cells would give the estimation of fibers that are attached to the cells. Background fluorescence that was measured from control experiments (unmodified cells) was subtracted from fluorescence intensity of fibers-attached cells, and values were plotted in FIG. 12. Similarly, the fluorescence intensity was assessed at different time points: day zero, after 1 and 2 days (FIG. 12). Results show that fluorescence intensity did not decreased significantly even after two days.

As another control free dye was used without encapsulating into the self-assembled fibers to rule out the possibility of non-specific bound dye on the cells. In these experiments, a similar procedure was used as described herein except using self-assembled fibers. Results are summarized in FIG. 12.

### Example 13

### Modification of hMSCs BY Self-Assembled Fibers in Suspension Mode

Loading of Dye Encapsulated Self-Assembled Fibers on hMSCs.

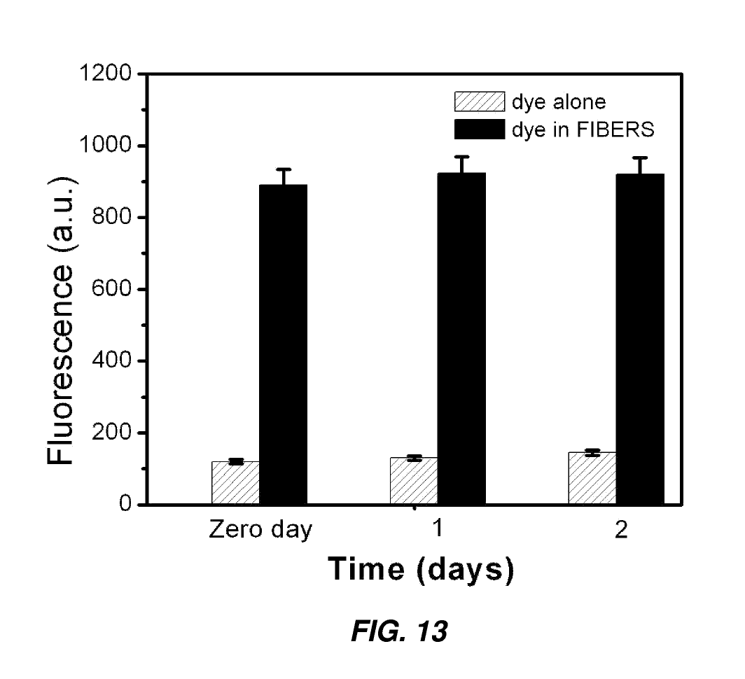

In suspension mode, a T75 flask up to 90% confluence in hMSC expansion media (15% Fetal Bovine Serum, 1% L-Glutamine, 1% Penn-Strep in a-MEM), were trypsinized using 1× trypsin-EDTA solution and then washed with phosphate buffer saline (PBS without Ca/Mg, pH 7.4) to remove media and trypsin. The cell pellet was then dispersed in 1 mL of dye encapsulated self-assembled fibers contain PBS, and incubated at room temperature for 30 min. After that the cells were centrifuged and spun down to remove unbound fibers. However residual amount of fibers were spun down with the cells. The cell pellets were washed with PBS twice by resuspending the cells followed by centrifugation and finally plated them in 24 well plates. After cells were adhered to the culture flask (3 hours), plates were thrice washed with PBS to remove residual unbound fibers. The cell-fiber conjugates were imaged using fluorescence microscopy where DiD give red emission. Quantifying the dye on cells would give an estimation of fibers that are attached to the cells. Background fluorescence that was measured from control experiments (unmodified cells) was subtracted from fluorescence intensity of fibers-attached cells, and values were plotted in FIG. 13. Similarly, we have followed the fluorescence intensity at different time points zero day, after 1 and 2 days (FIG. 13). Results show that fluorescence intensity did not decreased significantly even after two days.

As another control, free dye was used without encapsulating into the self-assembled fibers to rule out the possibility of non-specific bound dye on the cells. In these experiments, we have used similar procedure that is described in 00026 except using self-assembled fibers. Results are summarized in FIG. 13.

### Example 14

### Modification of hMSCS with Biotinylated Vesicles

### Preparation of Vesicles Using Biotinylated Lipid.

Chloroform solution of 1,2-Dioleoyl-sn-Glycero-3-Phosphoethanolamine-N-(Biotinyl) sodium salt (1 mg of lipid) was taken in glass vial and slowly chloroform was evaporated at high vacuum to make anhydrous thin film. The film was hydrated with 5 mL of PBS for 24 hours, subsequently, they were sonicated for 5 min and cooled in ice for 5 min. Similarly, freeze and thaw was repeated thrice to produce vesicles. Resulted vesicles were examined under transmission electron microscope and found that vesicles are spherical in shape and unilamellar in nature.

Modification of hMSCs with Biotinylated Vesicles

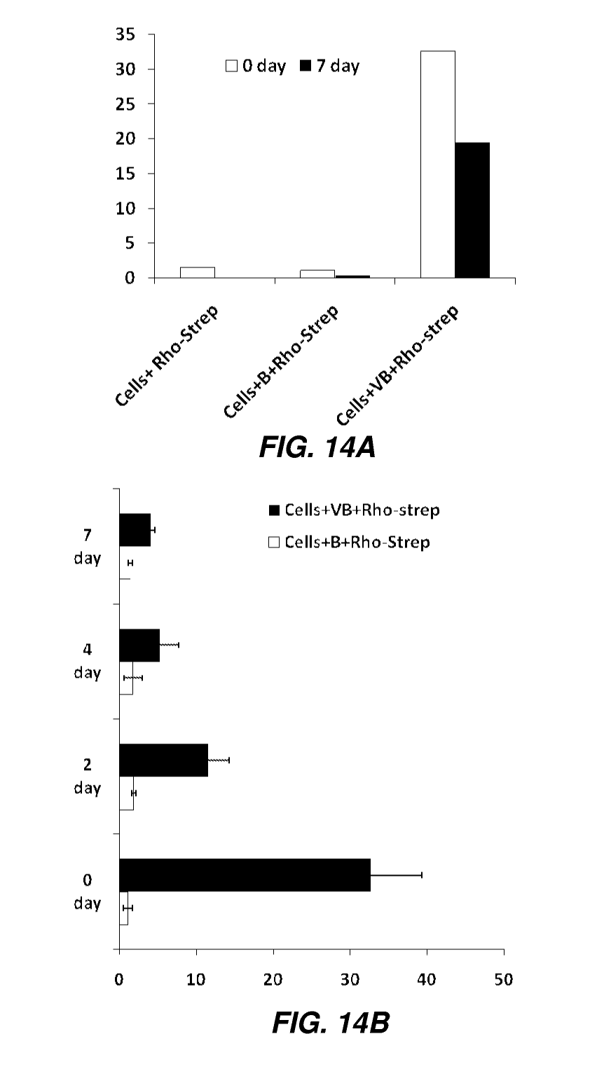

hMSCs were seeded in tissue culture wells of 96 well plates and were cultured in a cell expansion media until reaching 90% confluence. 0.5 mL of vesicle solution was added to the adherent hMSCs on tissue culture plates and incubated for 30 min and washed subsequently to remove excess of vesicles/lipids. The wells were rinsed with 300 μL of media thrice to remove excess vesicles/lipids. Subsequently, rhodamine-streptavidin solution (50 μg/mL in PBS without Ca/Mg) was added and incubated for 5 min. Then the cells were washed twice with PBS to remove unbound rhodamine-streptavidin. The cells were imaged using fluorescence microscopy where rhodamine-streptavidin gives red fluorescence. As a control experiment, hMSCs were treated with only biotin (without lipid molecule/vesicle-form) using similar procedure that described above (except using only biotin instead of vesicle solution). Results are summarized in FIG. 14A. Results show that biotinylated lipids are remained associated with the cells up to 7 days (FIG. 14A).

Accessibility of Biotin-Lipids on the Surface of hMSCs

To characterize the accessibility of the biotinylated lipids/vesicles on the cell surface after modifying the cells with vesicles, 200 μL of SR solution (50 μg/mL in PBS, pH 7.4 without Ca/Mg) was added for 20 minutes at room temperature on vesicles-modified cells after 0, 2, 4 and 7 days. After removal of excess streptavidin, the fluorescence intensity was quantified (FIG. 14B), results show that after 2 days of lipid insertion into the cells a portion of the biotinylated lipids were accessible for further conjugation with streptavidin.

### Example 15

### Induction of a Mesenchymal Stem Cell Rolling Response

### Materials and Methods:

### Materials

Primary human MSCs, isolated from human marrow of healthy consenting donors, were obtained from the Center for Gene Therapy at Tulane University. P-selectin was purchased from R&D systems (Minneapolis, Minn.) and biotinylated sialyl Lewis(x)-poly(acrylamide) (sialyl-LewisX-PAA-Biotin, BSLeX) was purchased from Glycotech (Gaithersburg, Md.). α-MEM, L-Glutamine and Penn-Strep were purchased from Invitrogen. Sulfonated biotinyl-N-hydroxy-succinimide, BNHS was purchased from Thermo Fisher Scientific (Piercenet, Rockford, Ill.) and Fetal Bovine Serum was purchased from Atlanta Biologicals. Biotin-4-Fluorescein was purchased from Molecular Probes (Eugene, Oreg.). Anti-Human Cutaneous Lymphocyte Antigen antibody (HECA-452), the secondary antibody (FITC Mouse Anti-Rat IgM), FITC CD90 and PE-Cy5 antibody were purchased from BD Biosciences. FACS-Buffer is PBS with 1% FBS. All other chemicals and reagents were purchased from Sigma Aldrich (St. Louis, Mo.) and were used without further purification unless specified.

### Mesenchymal Stem Cell Culture

Primary human MSCs were maintained in expansion medium that consisted of 15% Fetal Bovine Serum (selected for its ability to expand MSCs), 1% (v/v) L-Glutamine, 1% (v/v) Penn-Strep, and a-MEM. All experiments were performed using MSCs at passage number 4-6 where cells expressed high level MSC markers CD90 and CD29 (>99% cells), and did not express hematopoietic markers CD34 or CD45 (0% of cells) as observed from flow cytometry analysis (results not shown).

Surface Modification of MSCs with a Rolling Ligand

The conjugation of BSLeX to the MSC surface through biotin-streptavidin was performed in phosphate buffer saline (PBS, pH 7.4 without Ca/Mg) at room temperature. Typically, media was aspirated from 80-90% confluent T75 flasks and cells were trypsinized using 1× trypsin-EDTA solution, centrifuged into a pellet, and washed with PBS twice. The resulting cell pellet was dispersed in sulfonated biotinyl-N-hydroxy-succinimide, BNHS solution (1 mM, 1 mL), which was allowed to incubate for 1, 5, or 10 min at room temperature (Reaction STEP1). The cells were then pelleted, and washed with PBS twice by centrifugation to remove unattached and/or physically adsorbed BNHS from the cell surface. Streptavidin solution (50 μg/mL in PBS, 1 mL) was then used to treat the cells for 1, 5, or 10 min at room temperature (Reaction STEP2). The cells were then pelleted, and washed with PBS. To the streptavidin conjugated cells, BSLeX solution (5 μg/mL in PBS, 1 mL) was added, and the suspension was allowed to incubate for 5, 10, or 60 min at room temperature (Reaction STEP3). Finally, the cells were pelleted and washed with PBS.

### Flow Cytometry

Flow cytometry analysis were performed using a BD FACSCalibur analyzer (BD Biosciences, San Jose, Calif., USA) equipped with an air-cooled 15 mW, 488 nm argon-ion laser and photomultipliers with 530 nm, 585 nm and 661 nm bandpass filters. Data was analyzed using the software BD CellQuest and WinMDI. Viable cells were selected and fluorescence was displayed in a histogram (number of cells examined versus the log relative fluorescence intensity). Ten thousand events were recorded per measurement. PBS treated cells were used to normalize the background fluorescence.

Analysis and Improvement of the Cell Modification using Flow Cytometry

Chemical conjugation efficiency was monitored by flow cytometry using a dye-conjugated reagent (i.e. biotin-4-fluorescein) as a representative biotinylated ligand for the biotin-streptavidin bridge. The conjugation process was optimized to determine the maximum coupling efficiency (i.e. site density) at each reaction step by varying the incubation time (Table 1). It was first examined how the incubation time of the biotin-4-fluorescein in STEP 3 impacted the conjugation efficiency. Controls included adsorbed biotin-4-fluorescein following PBS incubation for STEP 1 and 2. After determining an effective reaction condition for STEP 3, it was next examined how the fluorescein signal was impacted by varying the incubation time for streptavidin in STEP 2. Controls included adsorbed streptavidin (no BNHS) followed by the same STEP 3 used in the experimental group. After determining an effective reaction condition for STEP 2, finally, the reaction time of BNHS was examined in STEP 1.

TABLE 2
Experimental Conditions Used to Assess Coupling Efficiencies.

| | Reaction | | |
|---|---|---|---|
| Optimization step | Step 1 | Reaction Step 2 | Reaction Step 3 |
| Biotin-4-Fluorescein | BNHS | Streptavidin | Biotin-4-Fluorescein |
| Experimental Group | 10 min | 10 min | 1, 5 & 10 min |
| Biotin-4-Fluorescein | PBS | PBS | Biotin-4-Fluorescein |
| Unmodified MSC | 10 min | 10 min | 1, 5 & 10 min |
| Control | | | |
| Streptavidin | BNHS | Streptavidin | Biotin-4-Fluorescein |
| Experimental Group | 10 min | 1, 5 & 10 min | 4 min |
| Streptavidin | PBS | Streptavidin | Biotin-4-Fluorescein |
| Unmodified MSC | 10 min | 1, 5 & 10 min | 4 min |
| Control | | | |
| BNHS | BNHS | Streptavidin | Biotin-4-Fluorescein |
| Experimental Group | 5, 10 & | 1 min | 4 min |
| | 60 min | | |

### Site Density Determination

The site density of the biotinylated SLeX ligand on the MSC surface was determined using the Quantum™ Simply Cellular® kit from Bangs Laboratories, Inc. (Fishers, Ind.) as directed. Beads with a defined concentration of (mouse antibody) binding sites were used to create a calibration curve that relates the recorded fluorescence to the number of antibodies. Purified Rat Anti-Human Cutaneous Lymphocyte Antigen antibody (HECA-452) and the secondary antibody (FITC Mouse Anti-Rat IgM) were used to detect immobilized SLeX on the cell surface. The unmodified PBS treated cells were incubated with the primary and secondary antibody, which was used as a control. The antibody binding capacity (ABC) values from this control group were subtracted from the values of the modified cells to normalize the data. Three samples of cells were modified with the optimized modification reaction described above that included incubation for 10 min BNHS, 1 min for streptavidin and 4 min for BSLeX. The SLeX site density was determined, assuming a one to one binding ratio of SLeX to the HECA-452 antibody and a one to one binding ratio between the primary and secondary antibody. The diameter of the MSCs was measured by automated cell counter Cellometer® Auto (Nexcelom Biosciences, MA) with 20 μL cell solution (in cell expansion media) in a disposable counting chamber. Average cell diameter of the MSCs was obtained from the software analysis of Cellometer® Auto.

### Stability and Accessibility of the Covalently Conjugated Biotin on the Cell Surface

To characterize the potential stability and accessibility of biotin conjugated to the cell surface through NHS chemistry, the fluorescence signal over time was examined following the addition of rhodamine conjugated streptavidin (SR) to BNHS modified cells. The incubation of SR with PBS treated or with biotin treated cells were used as controls. After the incubation with SR, the cells were fixed with formaldehyde solution (4% in PBS), treated with TRITON X solution (0.1% in PBS, 100 μL) and stained with DAPI (1 μg/mL in PBS, 100 μL). The fluorescence intensity of microscopic images was analyzed using Nikon quantitative software (NIS-Elements version 3) by examining the intensity of individual cells and the data were expressed as the fluorescence intensity per unit area of the cell.

### Viability, Adhesion Kinetics, and Proliferation Analysis

The viability, adhesion kinetics and proliferation of BSLeX-modified MSCs and control PBS-treated MSCs were examined. Briefly, the viability of the cells was examined immediately after modification (time 0) and after the cells were incubated within 6 well plates for 48 h using a trypan blue exclusion assay. Cell adhesion kinetics was quantified by measuring the number of adherent cells on the tissue culture surface after 10, 30, 90 and 150 minutes. Proliferation was quantified by counting the number of cells within a T25 flask for a 7 day period with light microscopy at 10× for ten random fields.

### Differentiation of Cells

Multi-lineage differentiation potential of the BSLeX modified and PBS treated cells was examined by incubating cells with osteogenic and adipogenic induction media followed by respective colorimetric histological staining. Cells were assayed for osteogenic differentiation and adipogenic differentiation using cell membrane associated alkaline phosphatase activity and Oil Red O staining, respectively.

### Secretion of Paracrine Factors: SDF-1, IGF-1 and PGE-2 Quantification

Enzyme-Linked Immuno Sorbent Assay (ELISA) kits (R&D systems, MN) were used to quantify the production of Stromal cell Derived Growth Factor-1 (SDF-1), Insulin like Growth Factor-1 (IGF-1) and Prostaglandin E2 (PGE2) by BSLeX modified MSCs and the PBS treated MSCs. Cell supernatants were collected from cultures of 1×10⁴ cells plated within the wells of 48 well plates. The standard ELISA protocol (according to manufacturer) was used for the measurement of SDF-1, IGF-1 and PGE2 in the culture supernatants after 24 hr of incubation of the cells at 37° C. and 5% CO₂ with 500 μL of MSC expansion media. All samples were run in duplicates from two independent experiments. The level of paracrine factors in the expansion media that contained 15% serum was also examined.

### Examination of MSC Surface Marker Expression Following Chemical Modification

The effect of the chemical modification on the expression of MSC surface markers was examined by flow cytometry analysis from the antibody staining of CD90, CD29 and CD49d at time 0 (immediately after modification) and after 24 hours. CD90 and CD29 are markers for MSCs and CD49d (VLA-4) is believed to be a homing ligand on the surface of MSCs31. The change in surface marker expression for the chemically modified MSCs was expressed in terms of relative fluorescence with respect to the unmodified (PBS treated) cells. Cells from 8 T-150 flasks were trypsinized, combined and split into six groups to ensure the same antigen density in all groups. Three groups were treated with the optimized conditions described above and controls included 3 groups of cells that were treated only with PBS (during all steps). Immediately after modification, 100,000 cells from each group were re-suspended in 100 μL FACS-Buffer and stained with 1 μL of CD90, 4 μL of CD29 or 4 μL of CD49d antibody solution for 20 minutes on ice. After washing one time with FACS-Buffer, the cells were re-suspended in 300 μl FACS-Buffer and analyzed by flow cytometry. Cells that were treated with BNHS or PBS (but not with antibodies) were plated and incubated for 24 hours in expansion media. Thereafter, the cells were trypsinized and 100,000 cells from each group were collected and stained with antibodies following the same protocol described above.

### Preparation of P-Selectin Surfaces

The well surfaces within a 6-well plate were coated with P-selectin solution (5 μg/mL in PBS, 1 mL) for 18 hours on a plate shaker at room temperature. All P-selectin surfaces were freshly prepared prior to the flow chamber assay.

### Flow Chamber Assay

For the analysis of cell velocities through the flow chamber, the cells were suspended in MSC expansion media (−10⁵ cells/mL) for the flow chamber assay. A circular parallel plate flow chamber (Glycotech, Gaithersburg, Md.) with 127 μm gasket thickness and a width of 2.5 mm was used. To monitor cell rolling, phase contrast microscopy (TE2000-U Inverted Nikon Microscope with a DS-Qil Monochrome Cooled Digital Camera) was utilized and images were recorded in a 10× field at 10 second intervals. The velocity of the cells was calculated by measuring the distance cells traveled within a 10 second interval. A cell was classified as rolling if it rolled for 10 seconds while remaining in the field of view and if it traveled at an average velocity less than 50% of the calculated free stream velocity of a non-interacting cell. The flux was calculated manually based on number of cells interacting with the substrate and remaining in the field view for 10 seconds. Both the firmly adhered cells and rolling cells were considered for the flux calculation. To assess the effect of shear rate, the rolling velocity and the flux were measured at shear stresses including 0.36, 0.72 and 1.89 dyne/cm².

### Statistical Analysis:

For multiple pairwise comparisons, a two-tailed Student's t test was used with the Bonferroni correction.

### Results:

### Stability and Accessibility of Biotin on Cell Surface

To examine the stability and accessibility of immobilized biotin and rhodamine-conjugated streptavidin (SR) on MSCs over cell culture, the fluorescence signal of SR was examined immediately following BNHS and SR treatment (day 0) and after 7 days following BNHS and SR treatment. Cells covalently modified with BNHS display a significantly higher rhodamine signal compared to the controls (MSCs incubated with only SR(SR) and MSCs treated with biotin (B) and SR (B+SR)) on both 0 and 7 days after modifications. This suggests that the covalently attached biotin on MSCs was stable for at least 7 days. Thus, the temporal stability and accessibility of the covalently conjugated biotin on the MSC surface was examined by quantifying the fluorescence signal of SR added to the cells after a delay of 1, 2, 4, and 7 days following BNHS treatment. The fluorescence intensity was stabilized and retained up to 7 days and was significantly higher than the controls (P<0.01). These data indicate that covalent immobilization of biotin with BNHS treatment resulted in a stable and accessible biotin on the cell surface.

Analysis and Optimization of Functionalization Efficiency using Flow Cytometry

The effect of the incubation time of biotin-4-fluorescein in the third step of the modification process was determined. The increase of incubation time of biotin-4-fluorescein beyond five minutes did not result in significant increase of the fluorescence of the experimental group. In contrast, the control exhibited increased fluorescence with the increase of biotin-4-fluorescein incubation.

The time for incubation of BNHS-modified MSCs with streptavidin (Step 2) had a significant effect on the amount of biotin-4-fluorescein attached in the subsequent step. Specifically, incubation with streptavidin for 1 minute resulted in stronger overall fluorescence indicated by a sharp peak. Longer incubation time (5 min and 10 min) with streptavidin resulted in a decreased fluorescence and a broader peak compared to the group where BNHS-modified MSCs were incubated with streptavidin for 1 minute. The fluorescence of the control group cells (PBS treated MSCs followed by incubation with streptavidin and biotin-4-fluorescein) increased with increasing the streptavidin incubation time.

The reaction time of BNHS with the MSCs for (Step 1) also influences the relative fluorescence which is introduced by the subsequent conjugation with streptavidin followed by biotin-4-fluorescein. The fluorescence signal reached a plateau at ˜10 min BNHS treatment after which there was no significant increase as reaction time was increased.

For further experiments conditions were used which led to the strongest and most uniform staining across the cell population. The peak of the experimental group represents these conditions: 10 min BNHS, 1 min streptavidin and 5 minutes biotin-4-fluorescein. The ABC of the immobilized BSLeX on the surface of the MSCs, using the reactions conditions consisting of 10 min BNHS, 1 min streptavidin and 4 minutes biotin-4-fluorescein, was determined to be ˜27201±13786. This ABC corresponds to a site density of ˜10±5 SLeX moieties/μm² with the average cell diameter is 15±4 μm.

### Characterization Cell Phenotype

The cell viability of BSLeX modified cells was not significantly affected compared to PBS treated cells as observed within 48 hours after cell modification. Specifically, 88±4% of the SLeX modified cells were viable immediately after coupling compared to 92±3% viability of PBS treated cells. After 48 hr culture, 90±2% of the SLeX modified cells were viable compared to 89±2% of PBS treated cells. This indicates that modification of MSCs with SLeX did not induce any substantial toxicity. The adhesion kinetics of SLeX modified MSCs on tissue culture polystyrene dishes at 10, 30, 90 and 150 minutes was compared with PBS treated cells. The modified cells exhibited similar adhesion kinetics compared to the controls. No differences were detected in the proliferation rates of the BSLeX modified MSCs and PBS treated cells or their ability to attain a confluent monolayer after 7 days.

### Differentiation Potential of the Modified Cells

The differentiation potential of BSLeX modified MSCs was examined by inducing differentiation of the cells under osteogenic and adipogenic culture conditions and was compared with PBS treated cells as control. Specifically, alkaline phosphatase (ALP) activity and Oil Red O (ORO) staining were used as indicators for osteogenic and adipogenic differentiation, respectively. No significant differences in ALP or ORO staining were observed between BSLeX modified cells and the PBS treated cells after the induction of osteogenic and adipogenic differentiation. Moreover, the BSLeX modified cells grown in MSC expansion media did not positively stain for ORO or ALP. These results indicate that the covalent modification of the MSC surface neither induce adipogenic or osteogenic differentiation nor impair the cell multilineage differentiation potential.

### Release of Paracrine Factors

The secretion of paracrine factors by the MSCs modified with BSLeX was examined by quantifying SDF-1, IGF-1 and PGE2 secretion in the culture media over a 24 h timeframe using ELISA assays. No significant difference was observed between the levels of expression of the paracrine factors for the MSCs modified with BSLeX compared to the PBS treated cells. IGF-1 and PGE2 were not detectable in expansion media containing 15% serum, however, SDF-1 was detected at ˜5% of the amount observed from MSCs after 24 h.

### The Impact of Chemical Modification on the Expression of MSC Surface Markers

The expression of CD90 and CD29 receptors on the MSC surface were reduced following the 3 STEP modification with BNHS, streptavidin, and BSLeX compared to PBS treated cells. Specifically, immediately after the modification the BSLeX conjugated MSCs show 84%, 75% and 40% of relative fluorescence compared to PBS treated cells for CD90, CD29, and CD49d respectively. After 24 hours, the fluorescence of the modified MSCs and PBS treated cells were similar indicating that the CD90, CD29 and CD49d surface receptors were restored after 24 hours and that the level of expression of these receptors was similar to unmodified MSC controls.

### Rolling of Modified MSCs on P-Selectin

The rolling based adhesion interaction of the BSLeX moiety conjugated on MSC surface with P-selectin was analyzed by the flow chamber assay. The specific interaction between SLeX of the modified MSCs and P-selectin reduced the velocity from ˜70 μm/s to ˜0.5 μm/s at a wall shear stress of 0.36 dynes/cm². The number of interacting cells within a 10 second interval increased from 20 cells/mm² for PBS treated MSCs to 150 cells/mm² for SLeX modified MSCs. As the shear stress was increased from 0.36 dynes/cm² to 1.89 dynes/cm², the velocity of the modified cells increased modestly from 0.5 μm/s to 2 μm/s and the flux remained largely constant between 150 cells/mm² and 140 cells/mm². In a previous study, the velocity was increased significantly beyond a shear stress of 0.72 dynes/cm².

### Example 16

### Rolling Response of a Modified Mesenchymal Stem Cell with SLeX Through Vesicles

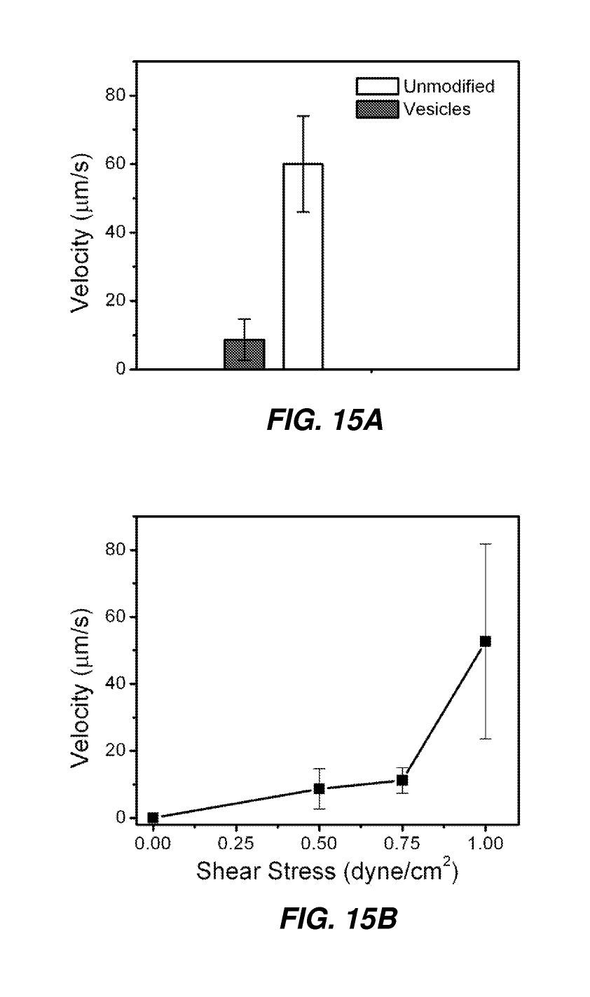

hMSCs were seeded in a T25 flask and were cultured in a cell expansion media until reaching 90% confluence. 1 mL of (1 mM) vesicle solution of 2-Dioleoyl-sn-Glycero-3-Phosphoethanolamine-N-(Biotinyl) sodium salt was added to the adherent hMSCs and incubated for 10 min and washed subsequently to remove excess of vesicles/lipids. The flask was rinsed with 1 mL of media thrice to remove excess vesicles/lipids. Subsequently, 1 mL of streptavidin solution (50 μg/mL in PBS without Ca/Mg) was added and incubated for 5 min. Then the cells were washed twice with PBS to remove unbound streptavidin. Subsequently, 1 mL of biotinylated sialyl Lewis X (SLeX) solution (5 μg/mL in PBS without Ca/Mg) was added and incubated for 5 min. The cells were washed twice with PBS to remove unbound SLeX. The cells were detached from the flask using 1 mL of non-enzymatic cell dissociation solution and washed once with 1 mL of PBS to remove the cell dissociation solution. The cells were then dispersed in 2 mL of cell expansion media and the rolling interaction was analyzed using a flow chamber assay. As a control experiment, hMSCs treated with PBS (unmodified cells) were used in the flow chamber assay. Results are summarized in FIGS. 15A-15B. Results show that hMSCs modified with SLeX in the adhesion mode through vesicles exhibits rolling interaction on P-selectin coated substrate. The vesicle modified cells exhibited a velocity of 7 μm/s at a shear stress of 0.5 dynes/cm² whereas the unmodified cell exhibited a velocity of 60 μM/s. FIG. 15B shows the effect of increasing shear stress on the SLeX modified hMSCs through vesicles.

### REFERENCES

All of the references cited herein and throughout the specification and examples are herein incorporated by reference in their entirety.
1. MacDiamid, J. A., et al., Bacterially derived 400 nm particles for encapsulation and cancer cell targeting of chemotherapeutics. Cancer Cell, 2007. 11(5): p. 431-45.
2. Studeny, M., et al., Bone marrow-derived mesenchymal stem cells as vehicles for interferon-beta delivery into tumors. Cancer Res, 2002. 62(13): p. 3603-8.
3. Stoff-Khalili, M. A., et al., Mesenchymal stem cells as a vehicle for targeted delivery of CRAds to lung metastases of breast carcinoma. Breast Cancer Res Treat, 2007. 105(2): p. 157-67.
4. Kumar, S, and S. Ponnazhagan, Bone homing of mesenchymal stem cells by ectopic {alpha}4 integrin expression. Faseb J, 2007.
5. Chamberlain, G., et al., Mesenchymal Stem Cells: their Phenotype, Differentiation Capacity, Immunological Features and Potential for Homing. Stem Cells, 2007.
6. Griffiths, L., et al., The macrophage—a novel system to deliver gene therapy to pathological hypoxia. Gene Ther, 2000. 7(3): p. 255-62.
7. Hwu, P., et al., In vivo antitumor activity of T cells redirected with chimeric antibody/T-cell receptor genes. Cancer Res, 1995. 55(15): p. 3369-73.
8. Harrington, K., et al., Cells as vehicles for cancer gene therapy: the missing link between targeted vectors and systemic delivery? Hum Gene Ther, 2002. 13(11): p. 1263-80.
9. Elgert, K. D., D. G. Alleva, and D. W. Mullins, Tumor-induced immune dysfunction: the macrophage connection. J Leukoc Biol, 1998. 64(3): p. 275-90.
10. Jang, J. H., T. L. Houchin, and L. D. Shea, Gene delivery from polymer scaffolds for tissue engineering. Expert Rev Med Devices, 2004. 1(1): p. 127-38.
11. Hill, E., T. Boontheekul, and D. J. Mooney, Regulating activation of transplanted cells controls tissue regeneration. Proc Natl Acad Sci USA, 2006. 103(8): p. 2494-9.
12. Leo, A. J. and D. A. Grande, Mesenchymal stem cells in tissue engineering. Cells Tissues Organs, 2006. 183(3): p. 112-22.
13. Sun X-D, L. I.-S., Gene technology in tissue engineering. American Journal of Biochemistry and Biotechnology, 2006. 2(2): p. 66-72.
14. Kemp, K. C., J. Hows, and C. Donaldson, Bone marrow-derived mesenchymal stem cells. Leuk Lymphoma, 2005. 46(11): p. 1531-44.
15. Gafni, Y., et al., Stem cells as vehicles for orthopedic gene therapy. Gene Ther, 2004. 11(4): p. 417-26.
16. Nakamura, K., et al., Antitumor effect of genetically engineered mesenchymal stem cells in a rat glioma model. Gene Ther, 2004. 11(14): p. 1155-64.
17. Pizzato, M., et al., Initial binding of murine leukemia virus particles to cells does not require specific Env-receptor interaction. J Virol, 1999. 73(10): p. 8599-611.
18. Zhuge, Y. and J. Xu, Racl mediates type I collagen-dependent MMP-2 activation. role in cell invasion across collagen barrier. J Biol Chem, 2001. 276(19): p. 16248-56.
19. Cheung, L. W., P. C. Leung, and A. S. Wong, Gonadotropin-releasing hormone promotes ovarian cancer cell invasiveness through c-Jun NH2-terminal kinase-mediated activation of matrix metalloproteinase (MMP)-2 and MMP-9. Cancer Res, 2006. 66(22): p. 10902-10.
20. Fujiuchi, Y., et al., Effect of hepatocyte growth factor on invasion of prostate cancer cell lines. Oncol Rep, 2003. 10(4): p. 1001-6.
21. Mohanam, S., et al., Increased invasion of neuroglioma cells transfected with urokinase plasminogen activator receptor cDNA. Int J Oncol, 1998. 13(6): p. 1285-90.
22. Farokhzad, O. C., J. M. Karp, and R. Langer, Nanoparticle-aptamer bioconjugates for cancer targeting. Expert Opin Drug Deliv, 2006. 3(3): p. 311-24.
23. Chithrani, B. D., A. A. Ghazani, and W. C. Chan, Determining the size and shape dependence of gold nanoparticle uptake into mammalian cells. Nano Lett, 2006. 6(4): p. 662-8.
24. Win, K. Y. and S. S. Feng, Effects of particle size and surface coating on cellular uptake of polymeric nanoparticles for oral delivery of anticancer drugs. Biomaterials, 2005. 26(15): p. 2713-22.
25. Schneider, G. B., et al., The effect of hydrogel charge density on cell attachment. Biomaterials, 2004. 25(15): p. 3023-8.
26. Sinclair, J. and A. K. Salem, Rapid localized cell trapping on biodegradable polymers using cell surface derivatization and microfluidic networking. Biomaterials, 2006. 27(9): p. 2090-4.
27. Saxon, E. and C. R. Bertozzi, Cell surface engineering by a modified Staudinger reaction. Science, 2000. 287(5460): p. 2007-10.
28. Huang, A., L. Huang, and S. J. Kennel, Monoclonal antibody covalently coupled with fatty acid. A reagent for in vitro liposome targeting. J Biol Chem, 1980. 255(17): p. 8015-8.
29. Dube, D. H. and C. R. Bertozzi, Metabolic oligosaccharide engineering as a tool for glycobiology. Curr Opin Chem Biol, 2003. 7(5): p. 616-25.
30. Hang, H. C. and C. R. Bertozzi, Ketone isosteres of 2-N-acetamidosugars as substrates for metabolic cell surface engineering. J Am Chem Soc, 2001. 123(6): p. 1242-3.
31. Sampathkumar, S. G., M. B. Jones, and K. J. Yarema, Metabolic expression of thiol-derivatized sialic acids on the cell surface and their quantitative estimation by flow cytometry. Nat Protoc, 2006. 1(4): p. 1840-51.
32. De Bank, P. A., et al., Surface engineering of living myoblasts via selective periodate oxidation. Biotechnol Bioeng, 2003. 81(7): p. 800-8.
33. Johnsson, N., N. George, and K. Johnsson, Protein chemistry on the surface of living cells. Chembiochem, 2005. 6(1): p. 47-52.
34. Toriello, N. M., E. S. Douglas, and R. A. Mathies, Microfluidic device for electric field-driven single-cell capture and activation. Anal Chem, 2005. 77(21): p. 6935-41.
35. Albretsen, C., et al., Applications of magnetic beads with covalently attached oligonucleotides in hybridization: isolation and detection of specific measles virus mRNA from a crude cell lysate. Anal Biochem, 1990. 189(1): p. 40-50.
36. Tiwari, A., et al., Magnetic beads (Dynabead) toxicity to endothelial cells at high bead concentration: implication for tissue engineering of vascular prosthesis. Cell Biol Toxicol, 2003. 19(5): p. 265-72.
37. Hinds, K. A., et al., Highly efficient endosomal labeling of progenitor and stem cells with large magnetic particles allows magnetic resonance imaging of single cells. Blood, 2003. 102(3): p. 867-72.
38. Lewin, M., et al., Tat peptide-derivatized magnetic nanoparticles allow in vivo tracking and recovery of progenitor cells. Nat Biotechnol, 2000. 18(4): p. 410-4.
39. Lacava, L. M., et al., Magnetic resonance of a dextran-coated magnetic fluid intravenously administered in mice. Biophys J, 2001. 80(5): p. 2483-6.
40. Wilson, K. M., et al., Single particle tracking of cell-surface HLA-DR molecules using R-phycoerythrin labeled monoclonal antibodies and fluorescence digital imaging. J Cell Sci, 1996. 109 (Pt 8): p. 2101-9.
41. Mistry, S. C., Iksoo; Messina, Darin Jeffrey; Gosiewska, Anna, Microparticles for cell delivery. 2005, JOHNSON & JOHNSON USA.
42. Halbert, G. W., Microparticles. 2007: USA.
43. Robert C., Klein, C., Cheng, G., Kogan, A., Mulligan, R. C., von Andrian, U. H., Cooper, T. S, Gene therapy to target dendritic cells from blood to lymph nodes. Gene Therapy 2003. 10(17):1479-86.

1. An isolated and engineered cell composition comprising
(a) a cell;
(b) a membrane associated ligand attached to a surface of said cell; and
(c) a particle attached to said surface of said cell.

2. The cell composition of claim 1, wherein said cell is a stem cell or a progenitor cell.

3-25. (canceled)

26. An isolated and engineered cell composition comprising,
(a) a cell; and
(b) a membrane associated ligand attached to a self-assembling molecule incorporated into a surface of said cell or a lipid molecule incorporated into a surface of said cell.

27-40. (canceled)

41. A method for forming an isolated and engineered cell composition, the method comprising the steps of:
(a) attaching a membrane associated ligand to a self-assembling molecule or amphiphilic molecule to form a modified molecule or modified amphiphilic molecule;
(b) forming a vesicle or micelle with said modified molecule or modified amphiphilic molecule; and
(c) fusing said vesicle or micelle with a cell.

42-72. (canceled)

73. An isolated and engineered cell composition comprising a cell and a membrane associated ligand attached to a surface of said cell, wherein said membrane associated ligand is attached to a first portion of said surface of said cell, and wherein a second portion of said surface of said cell is substantially free from said ligand or said ligand concentration is significantly reduced.

74-100. (canceled)

101. A method of treating an individual in need of targeted tissue regeneration, the method comprising the steps of:
(a) forming a targeted cell by attaching a membrane associated ligand to a surface of a cell, wherein said membrane associated ligand confers accumulation of said targeted cell in a tissue to be treated;
(b) forming a dual functional cell by attaching a particle to said surface of said targeted cell, wherein said particle comprises an agent, which enhances function of said dual functional cell; and
(c) administering said dual functional cell to an individual in need of targeted tissue regeneration.

102-110. (canceled)

111. A method for making a vaccine, the method comprising the steps of:
(a) forming a targeted antigen-presenting cell by attaching a membrane associated ligand to a surface of an antigen-presenting cell, wherein said membrane associated ligand confers accumulation of said targeted antigen-presenting cell in lymph tissue; and
(b) forming an activated targeted antigen-presenting cell by contacting said targeted antigen-presenting cell with an antigen; wherein said activated targeted antigen-presenting cell comprises a vaccine.

112-115. (canceled)

116. A kit for modifying a cell, the kit comprising:
(a) a self-assembling molecule with an attached moiety,
(b) instructions comprising a method for modifying a cell, and
(c) packaging materials therefor.

117-120. (canceled)

121. An isolated and engineered cell composition comprising
(a) a cell; and
(b) a particle attached to a surface of said cell; and
(c) a particle associated ligand not bound to said cell.

122-145. (canceled)

146. A method for preparing a cell composition in which a portion of the ligands are stable on the cell surface for at least 2 days after modification, the method comprising:
(a) preparing lipid vesicles comprising a ligand,
(b) contacting cells with said vesicles, wherein a cell composition is formed such that the ligand is present on the cell for at least 2 days after said contacting step.

147. (canceled)

148. A method for modifying a progenitor stem cell with a ligand without compromising stem cell characteristics, the method comprising covalently modifying the cell surface, wherein a cell composition is formed without loss of progenitor or stem cell characteristics.

149-150. (canceled)

151. A method for attaching an adhesion ligand to the surface of a cell, the method comprising the steps of:
(a) contacting a cell with a source of biotin,
(b) contacting said cell of step (a) with streptavidin and an adhesion ligand, wherein a cell composition is formed comprising an adhesion ligand on the cell surface and said adhesion ligand permits firm adhesion.

152-157. (canceled)

158. A method for attaching a particle to the surface of a cell, the method comprising:
(a) attaching a ligand to a particle to prepare a functionalized particle, and
(b) contacting said functionalized particle with a cell, wherein said functionalized particle is attached to the surface of said cell.

159-161. (canceled)

162. The method of claim 158, wherein said functionalized particle is internalized within the cell.

163. (canceled)

164. A composition comprising:
(a) an isolated cell; and
(b) a functionalized particle, wherein said functionalized particle is internalized within the cell.

165. The composition of claim 1, wherein the particle is 500-1000 nm in size.

166. The composition of claim 1, wherein the particle is 1000-8000 nm in size.

167. The composition of claim 1, wherein the particle comprises an agent.

168. The composition of claim 2, wherein the particle is 500-1000 nm in size.

169. The composition of claim 2, wherein the particle is 1000-8000 nm in size.

170. The composition of claim 2, wherein the particle comprises an agent.

171. The composition of claim 121, wherein the particle is 500-1000 nm in size.

172. The composition of claim 121, wherein the particle is 1000-8000 nm in size.

173. The composition of claim 121, wherein the particle comprises an agent.

174. The composition of claim 164, wherein the particle is 500-1000 nm in size.

175. The composition of claim 164, wherein the particle is 1000-8000 nm in size.

176. The composition of claim 164, wherein the particle comprises an agent.

177. The method of claim 101, wherein the particle is 500-1000 nm in size.

178. The method of claim 101, wherein the particle is 1000-8000 nm in size.

179. The method of claim 158, wherein the particle is 500-1000 nm in size.

180. The method of claim 158, wherein the particle is 1000-8000 nm in size.

181. The method of claim 162, wherein the particle is 500-1000 nm in size.

182. The method of claim 162, wherein the particle is 1000-8000 nm in size.
